SEQUENCE LISTING (3 sequences)

SEQ ID NO: 1 - DNA, 20 bp, Artificial Sequence
  Synthesized Sequence; gRNA Pacs1
catctcgcttaaggaaatga

SEQ ID NO: 2 - DNA, 20 bp, Artificial Sequence
  Synthesized Sequence; gRNA Pacs2
atgtgatctcaagacacgct

SEQ ID NO: 3 - DNA, 20 bp, Artificial Sequence
  Synthesized Sequence; gRNA Wdr37
gtgaaggacaagcgatcgat

(19) United States
(12) Patent Application Publication
(10) Pub. No.: US 2024/0043838 A1
(43) Pub. Date: Feb. 8, 2024

(54) COMPOSITIONS TARGETING WDR37 AND METHODS OF USE THEREOF

(71) Applicant: THE BOARD OF REGENTS OF THE UNIVERSITY OF TEXAS SYSTEM, Austin, TX (US)

(72) Inventors: Bruce Beutler, Dallas, TX (US); Evan D. Nair-Gill, Dallas, TX (US)

(73) Assignee: THE BOARD OF REGENTS OF THE UNIVERSITY OF TEXAS SYSTEM, Austin, TX (US)

(21) Appl. No.: 18/265,226

(22) PCT Filed: Dec. 2, 2021
(86) PCT No.: PCT/US2021/061602

§ 371 Date: Jun. 2, 2023

Related U.S. Application Data
(60) Provisional application No. 63/121,019, filed on Dec. 3, 2020.

(51) Int. Cl. C12N 15/113; A61K 45/06; A61P 37/06

(52) U.S. Cl. CPC C12N 15/113; A61K 45/06; A61P 37/06

(57) ABSTRACT

Compositions and methods for attenuating or preventing lymphoproliferation in a subject are provided. The subject may have, be suspected of having, or at risk of having a lymphoproliferative disease. The methods herein include administering to the subject a composition effective for decreasing WD repeat domain protein 37 (Wdr37) expression and/or activity.

### CROSS REFERENCE TO RELATED APPLICATIONS

This application is a national stage application filed under 35 U.S.C. § 371 of International Patent Application No. PCT/US2021/061602 entitled “COMPOSITIONS TARGETING WDR37 AND METHODS OF USE THEREOF” and filed on Dec. 2, 2021, which claims the benefit of U.S. Provisional Patent Application Ser. No. 63/121,019 entitled “WDR37 AS A TARGET FOR THE DEVELOPMENT OF DRUGS THAT TREAT B CELL LYMPHOMAS, B CELL LEUKEMIAS, AND AUTOIMMUNE DISORDERS” and filed on Dec. 3, 2020, the contents of which are hereby incorporated by reference in their entireties.

### ACKNOWLEDGEMENT OF GOVERNMENT SUPPORT

This invention was made with government support under Grant No. A1125581 awarded by the National Institutes of Health. The government has certain rights in the invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 4.0 kilobytes in size, and titled UTSD3611_SequenceListing_ST25.txt.

### BACKGROUND

### 1. Field

The present inventive concept is directed to compositions targeting Wdr37 (WD repeat domain 37) and methods of administering thereof for the treatment of a disease in a subject, such as a lymphoproliferative disease.

### 2. Discussion of Related Art

Lymphoproliferative diseases (LPDs) result from one or more defects within the immune system of a subject causing lymphocytes to be produced in excessive quantities. Two subsets of lymphocytes, T and B cells, divide uncontrollably in LPDs to produce immunoproliferative disorders, which are prone to immunodeficiency, a dysfunctional immune system, and lymphocyte dysregulation. Several gene mutations have been attributed as causes of LPD that can be iatrogenic or acquired. LPDs are also a recognized as a complication of primary immunodeficiency (PID) and immunodysregulatory syndromes with historically very poor patient outcomes. Accordingly, there is a need in the art for new targets for therapies toward LPDs.

### SUMMARY OF THE INVENTION

The present disclosure is based, at least in part, on the identification of Wdr37 as a treatment target within the immune system of a subject wherein inhibition and/or deletion of Wdr37 in a subject can block lymphoproliferation, a defect of which is associated with lymphoproliferative diseases (LPDs).

Certain embodiments of the present disclosure provide methods for treating, attenuating and/or preventing lymphoproliferation in a subject. In some embodiments, methods herein may comprise administering to the subject a composition effective for modulating WD repeat domain protein 37 (Wdr37). In some embodiments, modulating Wdr37 can comprise decreasing Wdr37 gene expression, decreasing Wdr37 protein expression, decreasing Wdr37 activity, or any combination thereof.

In certain embodiments, methods herein may comprise administering compositions effective for modulating Wdr37. In some embodiments, methods herein may comprise administering compositions effective for modulating Wdr37 wherein compositions herein may comprise at least one of a peptide, an antibody, a chemical, a compound, an oligo, a nucleic acid molecule, or any combination thereof. In some embodiments, a nucleic acid molecule herein can be a double-stranded RNA effective for inhibiting and/or decreasing expression of Wdr37 (e.g., gene expression of Wdr37, protein expression of Wdr37). In some embodiments, a double-stranded RNA herein can be small temporal RNA, small nuclear RNA, small nucleolar RNA, short hairpin RNA, microRNA, or any combination thereof. In some embodiments, a double-stranded RNA herein can be a small interfering RNA.

In certain embodiments, methods herein may comprise administering a composition effective for modulating Wdr37, wherein the composition may comprise at least one pharmaceutically acceptable excipient. In some embodiments, methods herein may comprise administering compositions disclosed herein to a subject topically, systemically, subcutaneously, intravenously, intranasally, or any combination thereof.

In certain embodiments, methods herein may comprise administration of a composition disclosed herein effective for modulating Wdr37 to a subject having, suspected of having, or at risk of having at least one lymphoproliferative disease, at least one lymphoid malignancy, or any combination thereof. In accordance with such embodiments, a subject having, suspected of having, or at risk of having at least one lymphoproliferative disease can be a human subject having one or more genetic markers for a lymphoproliferative disorder. In some embodiments, a human subject herein having one or more genetic markers for a lymphoproliferative disorder can be human subject that has been diagnosed as having or is suspected of having autoimmune lymphoproliferative syndrome (ALPS), Castleman disease (CD), Rosai-Dorfman disease (RDD), EBV-associated lymphoproliferative disorder (ELD), X-linked lymphoproliferative syndrome (XLP), angioimmunoblastic lymphadenopathy, caspase-8 deficiency syndrome (CEDS), Dianzani autoimmune lymphoproliferative disease, Kikuchi-Fujimoto syndrome, Llymphomatoid granulomatosis, lymphomatoid papulosis, ocular adnexal lymphoid proliferation, RAS-associated leukoproliferative disorder (RALD), p110δ activating mutation causing senescent T cells lymphadenopathy and immunodeficiency (PASLI), CTLA-4 haploinsufficiency with autoimmune infiltration (CHAI), LRBA deficiency with autoantibodies, regulatory T-cell defects, autoimmune infiltration and enteropathy (LATAIE), X-linked immunodeficiency with magnesium defect, EBV infection, and neoplasia (X-MEN), interleukin-2-inducible T-cell kinase (ITK) deficiency, or a combination thereof. In some embodiments, a subject administered compositions herein effective for modulating Wdr37 can be an immunocompromised subject. In some embodiments, an immunocompromised subject herein can be a human immunocompromised subject that has been diagnosed as having or is suspected of having common variable immunodeficiency (CVID), severe combined immunodeficiency (SCID), Wiskott-Aldrich syndrome, ataxia-telangiectasia, Chediak-Higashi syndrome, one or more viral infections, one or more fungal infections, or a combination thereof. In accordance with such embodiments, a human immunocompromised subject herein can be diagnosed as having or is suspected of having human immunodeficiency virus (HIV), severe acute respiratory syndrome coronavirus 1 (SARS-CoV-1), severe acute respiratory syndrome coronavirus 2 (SARS-CoV-2), Middle East Respiratory Syndrome (MERS), human coronavirus OC43 (HCoV-OC43), human coronavirus HKU1 (HCoV-HKU1), human coronavirus 229E (HCoV-229E), human coronavirus NL63 (HCoV-NL63), or any combination thereof. In some embodiments, a subject administered compositions herein effective for modulating Wdr37 can be a subject having, suspected of having, or at risk of having at least one lymphoid malignancy comprises a human subject having at least one lymphoid malignancy selected from the group comprising Hodgkin lymphomas, non-Hodgkin lymphomas, mature B cell neoplasms, mature T cell and natural killer (NK) cell neoplasms, and precursor lymphoid neoplasms. In some embodiments, a subject administered compositions herein effective for modulating Wdr37 may have undergone or may be undergoing at least one other therapy for lymphoproliferation. In accordance with such embodiments, an another therapy for lymphoproliferation herein can include administration of chemotherapy, rituximab, obinutuzumab, bortezomib, carfilzomib, azacitidine, decitabine, venetoclax, ibrutinib, idelalisib, sunitinib, dinaciclib, cobimetinib, idasanutlin, oblimersen sodium, sodium butyrate, depsipeptide, fenretinide, flavopiridol, gossypol, ABT-737, ABT-263, GX15-070, HA14-1, Antimycin A, acalabrutinib, zanubrutinib, tirabrutinib, bortezomib, lenalidomide, temsirolimus, or a combination thereof.

Certain embodiments of the present disclosure provide for compositions having at least one inhibitor of WD repeat domain protein 37 (Wdr37) and at least one pharmaceutically acceptable carrier. In some embodiments, compositions herein may further comprise at least one pharmaceutically acceptable excipient. In some embodiments, an inhibitor of Wdr37 as used herein can inhibit Wdr37 direct activity, inhibit Wdr37 indirect activity, inhibit formation of a complex between Wdr37 and phosphofurin acidic cluster sorting protein 1 (Pacs1), decrease expression of the Wdr37 gene, decrease expression of the Wdr37 protein, or any combination thereof. In some embodiments, an inhibitor of Wdr37 as disclosed herein can be a peptide, an antibody, a chemical, a compound, an oligo, a nucleic acid molecule, or a combination thereof. In some embodiments, an inhibitor of Wdr37 as disclosed herein can be a nucleic acid molecule having double-stranded RNA effective for inhibiting Wdr37 activity or decreasing the expression of Wdr37. In some embodiments, an inhibitor of Wdr37 as disclosed herein can be a double-stranded RNA selected from the group consisting of small temporal RNA, small nuclear RNA, small nucleolar RNA, short hairpin RNA and microRNA. In some embodiments, an inhibitor of Wdr37 as disclosed herein can be a small interfering RNA.

Certain embodiments of the present disclosure provide for methods of treating at least one lymphoproliferative disease, at least one lymphoid malignancy, or any combination thereof in a subject by administering and effective amount of a composition disclosed herein.

Certain embodiments of the present disclosure provide for kits having compositions disclosed herein and at least one container.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

Embodiments of the present inventive concept are illustrated by way of example in which like reference numerals indicate similar elements and in which:

FIGS. 1A-1J depict images illustrating that Pacs1 was required for normal numbers of circulating lymphocytes. FIG. 1A shows a super-pedigree mapping of two mutations in Pacs1 that were linked to peripheral B cell deficiency. Insert shows peripheral B cell deficiency in the endive and chicory pedigrees. Protein domain model shows the encoded location of the ENU alleles. Unpaired t test, ns=not significant, **P<0.01, and ***P<0.001. FIG. 1B shows a 1 base pair (bp) insertion in Pacs1 using CRISPR/Cas9 leads to loss of Pacs1 protein. FIG. 1C shows peripheral blood immune cell counts from Pacs1^{+/+} and Pacs1^{-/-} mice. Unpaired t test, *P<0.05, **P<0.01, and ***P<0.001. FIGS. 1D-1F show absolute numbers of lymphocytes subpopulations in the bone marrow (FIG. 1D), thymus (FIG. 1E), and spleen (FIG. 1F). B cell development in the bone marrow was assessed by FACS analysis for surface expression of: B220⁺CD43⁺CD19⁻IgM⁻IgD⁻ (pre-pro B); B220⁺CD43⁺CD19⁺IgM⁻D⁻ (pro B); B220⁺CD43⁻CD19⁺IgM⁻IgD⁻ (pre B); CD19⁺IgM⁺IgD⁻ (immature); CD19⁺IgM⁺IgD⁺ (mature). T cell development in the thymus was assessed by FACS analysis for surface expression of: CD4⁻CD8⁻ (double negative, DN); CD4⁺CD8⁺ (double positive, DP); CD4⁺CD8⁻ (CD4 single positive, SP); CD4⁻CD8⁺ (CD8 SP). Splenic B cell populations were assessed by FACS analysis for surface expression of: B220⁺CD21⁺CD23⁺ (follicular B cells, FOB); B220⁺CD21⁺CD23^{low} (marginal zone B cells, MZB). Each symbol represents an individual mouse. Mann-Whitney U test, ns=not significant, *P<0.05, **P<0.01, and ***P<0.001. FIGS. 1G-1I show a proportion of cell populations derived from Pacs1^{+/+}; CD45.1 and Pacs1^{-/-}; CD45.2 donors during competitive bone marrow reconstitution in the bone marrow (FIG. 1G), thymus (FIG. 1H), and spleen (FIG. 1I). Populations were determined based on the same markers as in FIG. 1C with the added congenic markers CD45.1 and CD45.2. Each symbol represents an individual recipient. Results are representative of two independent transplant experiments. FIG. 1J shows a measurement of cell death with Annexin V staining in FOB and MZB cells from the spleens of Pacs1^{+/+} and Pacs1^{-/-} mice. Results are representative of two independent experiments. Unpaired t test, ns=not significant, **P<0.01.

FIGS. 2A-2J depict images illustrating that Pacs1 deletion caused a defect in cytosolic Ca²⁺ flux after antigen receptor stimulation. FIGS. 2A-2F show Pacs1^{+/+} and Pacs1^{-/-} splenocytes labeled with Indo-1 and stained for B220, CD21, and CD23 to identify FOB (FIGS. 2A-2C) and MZB (FIGS. 2D-2E) cells. Fluorescence was measured for 30 seconds to establish a baseline and then cells were stimulated with the indicated amounts of anti-IgM (arrow). Cytosolic Ca²⁺ flux was monitored with FACS analysis by measuring the violet:blue fluorescence emission ratio of Indo-1. Kinetic traces are displayed from five independent Pacs1^{+/+} and Pacs1^{-/-} pairs and were normalized to baseline (Pacs1^{+/+} gray traces, Pacs1^{-/-} pink traces). The mean Ca²⁺ flux for each genotype is overlaid in bold (Pacs1^{+/+} black, Pacs1^{-/-} red). FIGS. 2G-2H show a maximum Ca²⁺ flux (peak height) at each anti-IgM concentration for FOB (FIG. 2G) and MZB (FIG. 2H) cells. Paired t test, ns=not significant, *P<0.05, **P<0.01, and ***P<0.001. FIG. 2I shows Pacs1^{+/+} and Pacs1^{-/-} FOB cells that were labeled with Indo-1 and stimulated in Ca²⁺ free buffer with 5 mcg/ml anti-IgM to assess ER Ca²⁺ efflux. Then, 2 mM Ca²⁺ was added back to assess SOCE. Kinetic traces normalized to baseline from three independent Pacs1^{+/+} and Pacs1^{-/-} pairs are shown with the mean Ca²⁺ flux overlaid in bold. FIG. 2J shows a maximum Ca²⁺ flux after stimulation under Ca²⁺ free conditions and after Ca²⁺ was added back. Paired t test, ns=not significant, *P<0.05.

FIGS. 3A-3K depict images illustrating that Wdr37 forms a mutually stabilizing complex with Pacs1. FIG. 3A shows a super-pedigree mapping of two mutations in Wdr37 that are linked to peripheral B cell deficiency. Insert shows peripheral B cell deficiency in radical and profound pedigrees. Unpaired t test, ns=not significant, ***P<0.001. FIGS. 3B and 3C show co-immunoprecipitation of HA-tagged Pacs1 by FLAG-Wdr37 (FIG. 3B) and HA-Wdr37 by FLAG-Pacs1 (FIG. 3C) in co-transfected 293T cells. FIG. 3D shows a Western blot for Pacs1 and Wdr37 expression in peripheral blood cells from WT, Pacs1^{-/-}, and Wdr37^{-/-} mice. FIG. 3E shows B and T cell peripheral blood counts in Wdr37^{-/-} mice. Unpaired t test, ***P<0.001. FIGS. 3F-3H show Wdr37^{+/+} and Wdr37^{-/-} splenocytes labeled with Indo-1, stained for cell surface markers to identify FOB cells, and stimulated with the indicated amounts of anti-IgM. Normalized traces from three (2.5 mcg/ml anti-IgM) or four independent experiments (10 mcg/ml and 5 mcg/ml anti-IgM) are shown (Wdr37^{+/+} gray, Wdr37^{-/-} pink). Mean Ca²⁺ flux for each genotype is overlaid in bold (Wdr37^{+/+} black, Wdr37^{-/-} red). FIG. 3/shows a maximum Ca²⁺ flux at each anti-IgM concentration. Paired t test, *P<0.05, **P<0.01. FIG. 3J shows Wdr37^{+/+} and Wdr37^{-/-} FOB cells labeled with Indo-1 and stimulated in Ca²⁺ free buffer with 5 mcg/ml anti-IgM followed by addition of 2 mM Ca²⁺. Normalized traces from four independent experiments are shown with mean Ca²⁺ flux overlaid in bold. FIG. 3K shows a maximum Ca²⁺ flux after stimulation under Ca²⁺ free conditions and after Ca²⁺ was added back. Paired t test, ns=not significant, *P<0.05.

FIGS. 4A-4G depict images illustrating that Pacs1 deletion induced ER stress, ROS, and heightened sensitivity to oxidative stress. FIG. 4A shows an immunoblot of ER mass, ER stress, and autophagy markers in Pacs1^{+/+} and Pacs1^{-/-} splenic B cells that were left unstimulated or stimulated overnight with 5 mcg/ml IgM. FIG. 4B shows B cells that were purified from Pacs1^{+/+} and Pacs1^{-/-} spleens and OCR was measured in unstimulated cells and in cells stimulated overnight with 5 mcg/ml anti-IgM. Data shown is the mean of 5-10 technical replicates from three (unstimulated) or four (stimulated) independent experiments. Unpaired t test, *P<0.05. FIGS. 4C-4D show a representative histogram of CelIRox Green staining in FOB cells from Pacs1^{+/+} and Pacs1^{-/-} spleens with MFI from three separate pairs of mice. Paired t test, **P<0.01. FIGS. 4E-4G show splenocytes from Pacs1^{+/+} and Pacs1^{-/-} mice stained with cell surface antibodies to identify FOB cells and treated with 100 mcM H₂O₂ for 35 minutes. Cells were then labelled with TMRE to monitor MMP by FACS analysis. Low TMRE fluorescence indicated susceptibility to H₂O₂ treatment. Data is presented as mean±SD. Results are representative of three independent experiments performed on different Pacs1^{+/+} and Pacs^{-/-} pairs.

FIGS. 5A-5E depict images illustrating that Pacs1^{-/-} B cells have reduced IP3R expression and ER Ca²⁺ stores. FIG. 5A shows an immunoblot of expression of all three IP3R isoforms and SERCA2 in primary splenic B cells from Pacs1^{+/+} and Pacs1^{-/-} mice. FIG. 5B shows real-time quantitative PCR of IP3R and SERCA2 transcripts from three independent Pacs1^{+/+} and Pacs1^{-/-} pairs of mice. Data is presented as mean±SD. FIG. 5C shows Pacs1^{-/-} FOB cells that were stimulated with 0.625 mcM thapsigargin under Ca²⁺-free conditions to measure intracellular Ca²⁺ stores. Kinetic traces of four independent experiments are shown (Pacs1^{+/+} gray, Pacs1^{-/-} pink) with the mean overlaid in bold (Pacs1^{+/+} black, Pacs1^{-/-} red). FIG. 5D shows a plateau of cytosolic Ca²⁺ flux from intracellular Ca²⁺ stores in FIG. 5C calculated by the mean value over the last 30 seconds of analysis. Paired t test, *P<0.05. FIG. 5E shows AUC of cytosolic Ca²⁺ flux from intracellular Ca²⁺ stores in FIG. 5C. Two-tailed paired t test, ns=not significant.

FIGS. 6A-6J depict images illustrating that Pacs1 deletion warped ER Ca²⁺ handling. FIG. 6A shows an immunoblot of Pacs1, Wdr37, IP3R1, and IP3R3 in the parental NIH-3T3 cell line and three separate Pacs1^{-/-} clones. FIG. 6B shows real-time quantitative PCR of IP3R isoform expression WT and Pacs1^{-/-} 3T3 cells. Expression in the Pacs1^{-/-} cells was measured in three independent clones. Data is presented as mean±SD. FIG. 6C shows Pacs1^{+/+} and Pacs1^{-/-} NIH-3T3 cells that were transfected with cytosolic aequorin and Ca²⁺ flux was measured after treatment with 1 mcM bradykinin. FIG. 6D shows a peak cytosolic Ca²⁺ concentration based on aequorin measurements in FIG. 6A. Unpaired t test, **P<0.01. FIG. 6E shows Pacs1^{+/+} and Pacs1^{-/-} NIH-3T3 cells (C1 and C2 from FIG. 6A) that were transfected with ER-GCamP6. ER Ca²⁺ was measured before and after treatment with 10 mcM ATP using the. Kinetic traces show the mean 488/405 excitation ratio of each cell line with error bars indicating SEM. Data is from 2 independent experiments. FIG. 6F shows ER Ca²⁺ release from the NIH-3T3 cell lines imaged in FIG. 6E. One-way ANOVA, **P<0.01, ***P<0.001. FIG. 6G shows basal ER Ca²⁺ levels from the NIH-3T3 cells imaged in FIG. 6E. One-way ANOVA, ns=not significant, ***P<0.001. FIG. 6H shows Pacs1^{+/+} and Pacs1^{-/-} 3T3 cells that were transfected with erAEQ then treated with tBHQ to measure ER Ca²⁺ leak. FIG. 6I shows a quantification of ER Ca²⁺ leak rate from FIG. 6H. Unpaired t test with Welch's correction *P<0.05. FIG. 6J shows ER Ca²⁺ leak linear regression.

FIGS. 7A-7P depict images illustrating spontaneous proliferation and increased cell death of Pacs1^{-/-} B cells in vivo under lymphocyte replete conditions. FIG. 7A shows Pacs1^{+/+} and Pacs1^{-/-} B cells that were purified, labeled with CTV dye, and stimulated with the indicated mitogens. Cell proliferation was assessed after 72 h with FACS analysis based on CTV dilution. FIGS. 7B and 7C show Pacs1^{+/+} and Pacs1^{-/-} mice that were immunized with alum-ova and one week later with NP-Ficoll. Anti-ova IgG and anti-NP IgM titers were measured at 14 days and 7 days after immunization, respectively. Each symbol represents an individual mouse. FIGS. 7D-7E shows Pacs1^{+/+} and Pacs1^{ccy/ccy} mice that were immunized with NP-KLH. Low affinity (anti-NP₃₀; FIG. 7D) and high affinity (anti-NP₂; FIG. 7E) antibodies were measured 14 days after immunization. FIGS. 7F-7L show B cells purified from Pacs1^{+/+} and Pacs^{-/-} mice and labeled with CTFR and CTV dyes, respectively. Labeled B cells were injected into unirradiated CD45.1 recipients at ˜1:1 ratio. Proliferation and survival of adoptively transferred B cells were measured 8 days post-transplant. FIG. 7M shows a fraction of donor B cells that proliferated after adoptive transfer from independent experiments using three different Pacs1^{+/+} and Pacs1^{-/-} F donor pairs. Unpaired t test, **P<0.01, ***P<0.001. FIG. 7N shows a fraction of donor B cells that were Annexin V positive after adoptive transfer from two independent experiments using two different Pacs1^{+/+} and Pacs1^{-/-} donor pairs. Unpaired t test, **P<0.01, ***P<0.001. FIGS. 7O and 7P show Pacs1^{+/+} and Pacs1^{-/-} mice that were injected with EdU and the fraction of EdU+FOB and MZB cells were measured in the spleen at 1, 4, and 7 days post-injection. Data from one independent experiment.

FIGS. 8A-8V depict images illustrating that Pacs1 deletion suppressed abnormal lymphocyte accumulation in models of lymphoproliferation. FIG. 8A shows spleen size and FACS analysis of abnormally expanded B220⁺CD23⁺CD21^{+/low} FOB cells in Pacs1^{+/-}; Bcl2^{TG} and Pacs1^{-/-}; Bcl2^{TG} mice. FIGS. 8B-8D shows the number of circulating B cells in the blood and FOB cells in the spleen of Pacs1^{+/-}; Bcl2^{TG} and Pacs1^{-/-}; Bcl2^{TG} mice. Mann-Whitney U test, *P<0.05, **P<0.01. FIGS. 8E-K show B cells that were purified from the spleens of Pacs1^{+/-}; Bcl2^{TG} and Pacs1^{-/-}l ; Bcl2^{TG} mice (CD45.2), labelled with CTFR and CTV proliferation dyes, respectively, and transplanted into unirradiated CD45.1 recipients. Donor B cells were measured in the spleen of recipient mice 7 days after B cell transfer based on CD45.2 expression and proliferation dye fluorescence. FIGS. 8L and 8M show fractions of proliferating (FIG. 8L) and recovered (FIG. 8M) donor cells from the experiment in FIGS. 8E-8K. Symbols represent individual recipient mice and data is from two independent adoptive transfer experiments. FIG. 8N shows a fraction of apoptotic B cells in the adoptively transferred B cell populations in the experiment in FIG. 8E-8K. Symbols represent individual recipient mice and data is from one adoptive transfer experiment. FIGS. 8O-8Q show splenocytes from Pacs1^{+/-}; Bcl2^{TG} and Pacs1^{-/-}; Bcl2^{TG} mice that were stained with cell surface antibodies to identify FOB cells and treated with 100 mcM H₂O₂ for 35 minutes. Cells were then labelled with TMRE to monitor MMP. TMRE fluorescence was measured by FACS analysis. Data is presented as mean±SD. Results are from one independent experiment. FIGS. 8R-8T show lymph node size and flow cytometry of lymphoproliferative CD3⁺B220⁺ cells in Pacs1^{+/+}; Fas^{Ipr/Ipr} and Pacs1^{-/-}; Fas^{Ipr/Ipr} or mice. FIGS. 8U-8V show enumeration of CD3⁺B220⁺ cells in the peripheral blood and lymph nodes of Fasor i or dependent on Pacs1 expression. Mann-Whitney U test, **P<0.01.

FIGS. 9A and 9B depict images illustrating creation of mice used in some examples. FIG. 9A shows Pacs1 expression in splenocytes from Pacs1^{+/+} and Pacs1^{ccy/ccy} mice. FIG. 9B shows a gene model for 1 bp insertion into exon 4 of Pacs1 using CRISPR/Cas9 to generate Pacs1^{-/-} mice.

FIGS. 10A-10J depict images illustrating ER Ca2+ efflux in Pacs1−/− lymphocytes after antigen receptor stimulation. FIGS. 10A and 10B show splenocytes from Pacs1^{+/+} and Pacs1^{-/-} mice that were stained for CD8 and CD4 and labeled with Indo-1. Cells were then stimulated with 10 mcg anti-CD3. Cytosolic Ca²⁺ flux was monitored by FACS analysis. Kinetic traces are displayed from three independent Pacs1^{+/+} and Pacs1^{-/-} pairs and were normalized to baseline (Pacs1^{+/+} gray traces, Pacs1^{-/-} pink traces). The mean Ca²⁺ flux for each genotype is overlaid in bold (Pacs1^{+/+} black, Pacs1^{-/-} red). FIGS. 10C and 10D show maximum Ca²⁺ flux in CD8 and CD4 T cells after anti-CD3 stimulation. Paired t test, *P<0.05. FIGS. 10E and 10F show stimulation of CD8 and CD4 T cells with 10 mcg anti-CD3 under Ca²⁺-free conditions followed by addition of 2 mM Ca²⁺. FIGS. 10G-10J show peak of Ca²⁺ flux in CD8 and CD4 T cells under Ca²⁺-free conditions and after addition of 2 mM Ca²⁺. Paired t test, *P<0.05, **P<0.01.

FIGS. 11A-11J depict images illustrating Pacs1^{-/-} B cell deficiency and Ca²⁺ flux phenotypes. FIGS. 11A-11B show the total number of B cell subpopulations in Pacs1^{+/+} and Pacs1^{-/-} mice harboring the B-18i heavy chain transgene. Unpaired t test, ns=not significant, *P<0.05, **P<0.01. FIGS. 11C-11D show identification of NP-specific FOB cells in spleens from Pacs1^{+/+}; IgH^{B-18i/+} and Pacs1^{-/-}; IgH^{B-18i+} mice using NP-PE. FIGS. 11E-11F show Ca²⁺ flux kinetic traces within the NP+ and NP− gates after treatment with NP-PE and then with anti-IgM from three independent experiments (Pacs1^{+/+}; IgH^{B-18i/+} are gray traces, Pacs1^{-/-}; IgH^{B-18i/+} are red/pink traces). Traces are normalized to baseline. FIGS. 11G-11J show maximum Ca²⁺ flux peak height after each stimulation within the NP+ and NP− gates. Paired t test, * P<0.05, **P<0.01, ***P<

FIG. 12 depicts an image illustrating signaling upstream of ER Ca²⁺ release in Pacs^{-/-} B cells. B cells were purified from the spleens of Pacs1^{+/+} and Pacs1^{-/-} mice and stimulated with 5 mcg/ml of anti-IgM for the indicated times. Phosphorylated and total amounts of Plcγ2, ERK, and AKT were measured by Western blot.

FIGS. 13A-13C depict images illustrating Wdr37 forming a mutually stabilizing complex with Pacs1. FIG. 13A shows a measurement of Pacs1-dependent Wdr37 expression in lymphoid tissues from Pacs1^{+/+} and Pacs1^{-/-} mice. FIG. 13B shows a measurement of mutual stabilization of epitope-tagged Pacs1 and Wdr37 in 293T cells after CXH treatment. FIG. 13C shows a gene model for 2 bp deletion from exon 4 of Wdr37 using CRISPR/Cas9 to generate Wdr37- mice.

FIGS. 14A-14E depict images illustrating proportions of circulating B cells in Pacs2^{-/-} mice. FIGS. 14A-14B show gene models for Pacs2 deletion using CRISPR/Cas9. Exon 3 of Pacs2 was targeted, generating 20 bp deletion (FIG. 14A) and 1 bp insertion (FIG. 14B) frameshifting alleles. These alleles were predicted to result in early truncation of Pacs2. FIG. 14C shows a measurement of the proportion of B220+ B cells in the peripheral blood of Pacs2^{-/-} mice. Red symbols represent mice carrying the 20 bp deletion allele and blue symbols represent mice carrying the 1 bp insertion allele. FIG. 14D shows Pacs1 and Wdr37 expression in primary splenocytes from WT, Pacs1^{-/-}, and Pacs2 mice. FIG. 14E shows splenocytes from Pacs2^{+/+} and Pacs2^{-/-} mice that were loaded with Indo-1 and stained to identify FOB cells. Cells were stimulated with 5 mcg of anti-IgM and cytosolic Ca²⁺ flux was monitored by FACS analysis. Results are representative of two independent experiments.

FIGS. 15A-15H depict images illustrating Pacs1 deletion effects on mitochondrial Ca²⁺ homeostasis. FIG. 15A shows Pacs1^{+/+} and Pacs1^{-/-} 3T3 cells that were transfected with erAEQ then treated with 1 mcM bradykinin to measure ER Ca²⁺ release. FIG. 15B shows a quantification of ER Ca²⁺ release rate from (A). Unpaired t test *** P<0.001. FIG. 15C shows Pacs1^{+/+} and Pacs1^{-/-} NIH-3T3 cells that were infected with MSCV-Mito-Pericam. Mitochondrial Ca²⁺ flux was measured before and after treatment with 10 mcM ATP with live cell imaging using the 488/405 excitation ratio. Each trace shows the kinetic of individual cells (Pacs1^{+/+} gray, Pacs1^{-/-} pink) with the mean overlaid in bold (Pacs1^{+/+} black, Pacs1^{-/-} red). Results are representative of two independent experiments. FIG. 15D shows a maximum mitochondrial Ca²⁺ flux from the cells measured in (C). Mann-Whitney U test, ***P<0.001. FIG. 15E shows Pacs1^{+/+} and Pacs1^{-/-} NIH-3T3 cells that were transfected with mt2-GCamP and basal mitochondria Ca²⁺ content was measured. Mann-Whitney U test, ns=not significant. Results are combined from two independent experiments. FIG. 15F-15G show Pacs1+/+ and Pacs1−/− splenocytes stained to identify FOB cells then labeled with MitoTracker Green. Histogram shows representative intensity of MitoTracker fluorescence in FOB cells (FIG. 15F). Quantification shows the results of two pairs of Pacs1+/+ and Pacs1−/− mice (FIG. 15G). Each symbol represents the mean value from three technical replicates. Horizontal bars indicate mean values from the combined experiments. FIG. 15H shows a mitochondrial stress test of purified Pacs1+/+ and Pacs1−/− B cells. Symbols represent the mean of three independent experiments each with 7-10 technical replicates. Error bars show SD between the combined experiments. Two-tailed unpaired t test. *P<0.05, ns=not significant.

FIG. 16 depicts an image illustrating Pacs1^{+/ +} and Pacs1^{-/-} splenic B cells that were labeled with CTV and either left unstimulated or stimulated with the indicated homeostatic cytokines and mitogens.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based on, in part, the suppressing discovery that Wdr37 is important in immunoregulation and regulates frequencies of peripheral blood B cells, IgD+B cells, and IgM+B cells. Prior to the present disclosure, Wdr37 had no known physiological function. Exemplary methods herein showed that Wdr37 deletion resulted in defective endoplasmic reticulum (ER) calcium (Ca²⁺) efflux in B and T cells after antigen receptor stimulation. Exemplary methods herein also showed that Wdr37 deletion did not impair normal humoral responses, but it strongly blocked lymphoproliferation that resulted from Fas^{Ipr} mutation and Bcl2 overexpression. Accordingly, the present disclosure herein provides a novel target, Wdr37, for therapies aimed toward suppressing LPDs while preserving beneficial immune functions. In certain embodiments, the present disclosure herein provides compositions for targeting Wdr37. In certain embodiments, the present disclosure herein provides methods of administering compositions for targeting Wdr37 to a subject in need thereof. In certain embodiments, the present disclosure herein provides methods of preventing, treating, and/or attenuating a disease resulting from Pacs1-Wdr37 complex control of lymphocytes (e.g., LPDs).

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. Compositions

### (a) Wdr37

In certain embodiments, compositions herein can modulate Wdr37 (WD repeat domain 37). As used herein, compositions “modulating” Wdr37 can include any biomolecule(s) capable of decreasing Wdr37 gene expression, decreasing Wdr37 protein expression, decreasing Wdr37 activity, preventing formation of a Wdr37-Pacs1 complex, or a combination thereof. In some embodiments, biomolecule(s) capable of modulating Wdr37 can be a peptide, and antibody, a chemical, a compound, an oligo, a nucleic acid molecule, or a combination thereof. In some embodiments, biomolecule(s) herein capable of modulating Wdr37 can be an inhibitor of Wdr37. As used herein, an inhibitor of Wdr37 can inhibit Wdr37 direct activity, inhibit Wdr37 indirect activity, inhibit formation of a Wdr37-Pacs1 complex, decrease expression of the Wdr37 gene, decrease expression of the Wdr37 protein, or a combination thereof.

WD repeat domain protein 37 (Wdr37) is an uncharacterized protein belonging to the WD repeat protein family. WD proteins typically function as scaffolds for macromolecular signaling complexes and are implicated in diverse cellular processes. Accordingly, some embodiments herein can include modulators and/or inhibitors of targets upstream or downstream of the Wdr37 signaling cascade that could effectively inhibit the physiological outcome of Wdr37 inhibition. The WD repeat protein family is characterized by 40 amino acid repeats bracketed by glycine-histidine and tryptophan-aspartic acid (GH-WD) residues which fold into 7-bladed beta-propellers. Accordingly, some embodiments herein can include modulators and/or inhibitors that target at least one propeller-like region of Wdr37.

In certain embodiments, compositions herein can include modulators and/or inhibitors of Wdr37. In some embodiments, modulators and/or inhibitors of Wdr37 can be peptides, antibodies, chemicals, compounds, oligos, nucleic acid molecules, or a combination thereof.

In certain embodiments, modulators and/or inhibitors of Wdr37 disclosed herein can be used to treat, attenuate, or prevent a lymphoproliferative disease. In certain embodiments, modulators and/or inhibitors of Wdr37 disclosed herein can be used to treat, attenuate, or prevent lymphoid malignancy. In certain embodiments, modulators and/or inhibitors of Wdr37 disclosed herein can be used to attenuate over-proliferation of lymphocytes. In certain embodiments, modulators and/or inhibitors of Wdr37 disclosed herein can be used to attenuate over-proliferation B cells, T cells, or any combination thereof.

In certain embodiments, compositions herein can include a nucleic acid molecule. The term “nucleic acid molecule” as used herein refers to a molecule having nucleotides. The nucleic acid can be single, double, or multiple stranded and may comprise modified or unmodified nucleotides or non-nucleotides or various mixtures and combinations thereof. In some embodiments, a nucleic acid molecule for use herein can be a double-stranded RNA. In some embodiments, a double stranded RNA suitable for use herein can be small temporal RNA, small nuclear RNA, small nucleolar RNA, short hairpin RNA, microRNA, or the like. In certain embodiments, a double stranded RNA suitable for use herein can be a small interfering RNA.

In accordance with the present disclosure, small interfering RNA against specific mRNAs produced in the affected cells may prevent the production of the disease related proteins in targeted cells (e.g., Wdr37). In certain embodiments, compositions herein may comprise the use of one or more specifically tailored vectors designed to deliver small interfering RNA to targeted cells. In some embodiments, the success of the designed small interfering RNAs herein may be predicated on their successful delivery to the targeted cells to treat lymphoproliferative diseases. In some embodiments, small interfering RNAs herein may be capable of targeting specific mRNA molecules in human cells. In some embodiments, small interfering RNA vectors herein can be constructed to transfect cells and produce small interfering RNA that cause the cleavage of the target RNA and thereby interrupt production of the encoded protein. In some embodiments, a small interfering RNA vector of the present disclosure may prevent production of the target protein (e.g., Wdr37) by suppressing production of the protein itself, by suppressing production of a protein involved in the production or processing of the target protein, or a combination thereof.

In certain embodiments, a small interfering RNA vector of the present disclosure can prevent production of Wdr37 in a cell. In some embodiments, a small interfering RNA vector of the present disclosure can attenuate production of Wdr37 in a cell. In some embodiments, production of Wdr37 in a cell can be attenuated by at least 25% using a small interfering RNA vector disclosed herein. In some embodiments, production of Wdr37 in a cell can be attenuated by about 10% to about 99% (e.g., about 10%, about 20%, about 30%, about 40%, about 50%, about 60%, about 70%, about 80%, about 90%, about 99%) using a small interfering RNA vector disclosed herein.

An anti-Wdr37 small interfering RNA disclosed herein, as well as the other small interfering RNAs for treating, attenuating and preventing lymphoproliferation, are just but some examples of the embodiment of the present disclosure. In some embodiments, screening using the screening platforms disclosed herein may be used to identify one or more additional candidate small interfering RNAs for use herein.

In some embodiments, a nucleic acid molecule disclosed herein can be used to genetically modulate gene expression of Wdr37 in a targeted cell. As used herein, the term “genetically modulate” refers to manipulation of an immune cell genome using genetic engineering techniques. Non-limiting examples of genetic engineering techniques that can be used to modulate gene expression of Wdr37 in a target cell can include chemical mutagenesis, x-ray mutagenesis, recombinant DNA techniques, virus-mediated delivery of DNA, gene editing, and the like. Examples of gene editing methods include, but are not limited to, CRISPRs, TALENs, Zinc Finger Nucleases, and the like. In some embodiments, CRISPR can be used to modulate gene expression of Wdr37 in a target cell.

In certain embodiments, modulators and/or inhibitors of Wdr37 disclosed herein can be packaged in a vector for delivery to a target cell. In some embodiments, a vector for use herein may be an adeno-associated virus (AAV). In some embodiments, and AAV for us herein may be recombinant adeno-associated virus serotype 2 and/or recombinant adeno-associated virus serotype 5. Alternatively, other viral vectors, such as herpes simplex virus, can be used for delivery of foreign DNA to central nervous system neurons herein. In some embodiments, non-viral vectors, such as but not limited to, plasmid DNA delivered alone or complexed with liposomal compounds or polyethyleneamine may be used herein to deliver modulators and/or inhibitors of Wdr37 disclosed herein to the target cell or tissue.

In certain embodiments, modulators and/or inhibitors of Wdr37 disclosed herein may be administered directly, or may be complexed with cationic lipids, packaged within liposomes, packaged within viral vectors, or otherwise delivered to target cells or tissues. In some embodiments, complexes comprising modulators and/or inhibitors of Wdr37 herein can be locally administered to relevant tissues ex vivo, or in vivo through injection, infusion pump or stent, with or without their incorporation in biopolymers.

In certain embodiments, the present disclosure provides mammalian cells containing one or more nucleic acid molecules and/or expression vectors disclosed herein. The one or more nucleic acid molecules may independently be targeted to the same or different sites.

In certain embodiments, modulators and/or inhibitors of Wdr37 of the present disclosure, individually, or in combination or in conjunction with other drugs, may be used to treat one or more disorders and/or diseases. In some embodiments, modulators and/or inhibitors of Wdr37 herein, individually, or in combination or in conjunction with other drugs, may be used to treat one or more genetic lymphoproliferative disorders. Examples of such diseases include, but are not limited to, autoimmune lymphoproliferative syndrome (ALPS), Castleman disease (CD), Rosai-Dorfman disease (RDD), EBV-associated lymphoproliferative disorder (ELD), X-linked lymphoproliferative syndrome (XLP), angioimmunoblastic lymphadenopathy, caspase-8 deficiency syndrome (CEDS), Dianzani autoimmune lymphoproliferative disease, Kikuchi-Fujimoto syndrome, Llymphomatoid granulomatosis, lymphomatoid papulosis, ocular adnexal lymphoid proliferation, RAS-associated leukoproliferative disorder (RALD), p110δ activating mutation causing senescent T cells lymphadenopathy and immunodeficiency (PASLI), CTLA-4 haploinsufficiency with autoimmune infiltration (CHAI), LRBA deficiency with autoantibodies, regulatory T-cell defects, autoimmune infiltration and enteropathy (LATAIE), X-linked immunodeficiency with magnesium defect, EBV infection, and neoplasia (X-MEN), interleukin-2-inducible T-cell kinase (ITK) deficiency, and the like.

In certain embodiments, modulators and/or inhibitors of Wdr37 herein, individually, or in combination or in conjunction with other drugs, can be used to treat an immunocompromised subject. In some embodiments, immunocompromised subjects to be treated with compositions disclosed herein can be diagnosed as having or can be suspected of having common variable immunodeficiency (LVID), severe combined immunodeficiency (SCID), Wiskott-Aldrich syndrome, ataxia-telangiectasia, Chediak-Higashi syndrome, one or more viral infections, one or more fungal infections, or any combination thereof. Examples of such viral infections include, but are not limited to human immunodeficiency virus (HIV), severe acute respiratory syndrome coronavirus 1 (SARS-CoV-1), severe acute respiratory syndrome coronavirus 2 (SARS-CoV-2), Middle East Respiratory Syndrome (MERS), human coronavirus 0043 (HCoV-0043), human coronavirus HKU1 (HCoV-HKU1), human coronavirus 229E (HCoV-229E), human coronavirus NL63 (HCoV-NL63), or any combination thereof.

In certain embodiments, modulators and/or inhibitors of Wdr37 herein, individually, or in combination or in conjunction with other drugs, can be used to treat subjects having, suspected of having, or at risk of having at least one malignancy. In some embodiments, modulators and/or inhibitors of Wdr37 herein, individually, or in combination or in conjunction with other drugs, can be used to treat subjects having, suspected of having, or at risk of having at least one lymphoid malignancy. Examples of such lymphoid malignancies include, but are not limited to Hodgkin lymphomas, non-Hodgkin lymphomas, mature B cell neoplasms, mature T cell and natural killer (NK) cell neoplasms, precursor lymphoid neoplasms, and the like. In some embodiments, modulators and/or inhibitors of Wdr37 of the present disclosure, individually, or in combination or in conjunction with other drugs, can be used to treat subjects having, suspected of having, or at risk of having at least one B cell lymphoma.

In certain embodiments, modulators and/or inhibitors of Wdr37 of the present disclosure, individually, or in combination or in conjunction with other drugs, can be used to treat subjects having, suspected of having, or at risk of having at least one type of leukemia. In some embodiments, a subject suitable for treatment herein can have acute leukemia or chronic leukemia. In some embodiments, a subject suitable for treatment herein can have lymphocytic leukemia or myelogenous leukemia. In some embodiments, a subject suitable for treatment herein can have Acute lymphocytic leukemia (ALL), Acute myelogenous leukemia (AML), Chronic lymphocytic leukemia (CLL), Chronic myelogenous leukemia (CML), hairy cell leukemia, or a rare, unnamed type of leukemia. In some embodiments, a subject suitable for treatment herein can have B cell leukemia.

### (b) Pharmaceutical Formulations and Treatment Regimens

In certain embodiments, modulators and/or inhibitors of Wdr37 disclosed herein may be provided per se or as part of a pharmaceutical composition, where the Wdr37 modulators and/or inhibitors can be mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptide, and antibody, a chemical, a compound, an oligo, a nucleic acid molecule, or a combination thereof toward modulating and/or inhibiting Wdr37 accountable for the biological effect. The term “active ingredient” as used herein can also include a genetically modified cell (e.g., stem cell, CAR T cell) as disclosed herein.

### (i) Pharmaceutically Acceptable Carriers and Excipients

Hereinafter, the phrases “physiologically acceptable carrier” and “pharmaceutically acceptable carrier” are interchangeably used herein to refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some embodiments, pharmaceutical compositions herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some embodiments, any of the well-known techniques, carriers, and excipients may be used as suitable and/or as understood in the art.

In certain embodiments, pharmaceutical compositions described herein may be an aqueous suspension comprising one or more polymers as suspending agents. In some embodiments, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some embodiments, pharmaceutical compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some embodiments, pharmaceutical compositions disclosed herein may comprise about 5% to about 99%, about 10%, about 95%, or about 15% to about 90% total amount of polymers as suspending agent(s) by total weight of the composition.

In certain embodiments, pharmaceutical compositions disclosed herein may comprise a viscous formulation. In some embodiments, viscosity of composition herein may be increased by the addition of one or more gelling or thickening agents. In some embodiments, compositions disclosed herein may comprise one or more gelling or thickening agents in an amount to provide a sufficiently viscous formulation to remain on treated tissue. In some embodiments, pharmaceutical compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some embodiments, pharmaceutical compositions disclosed herein may comprise about 5% to about 99%, about 10%, about 95%, or about 15% to about 90% total amount of gelling or thickening agent(s) by total weight of the composition. In some embodiments, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, pharmaceutical compositions disclosed herein may comprise at least 5%, at least 10%, at least 20%, at least 25%, at least 30%, at least 35%, at least 40%, at least 45%, or at least 50% total amount of one or more agents by total weight of the composition. In some embodiments, pharmaceutical compositions disclosed herein may comprise about 5% to about 99%, about 10%, about 95%, or about 15% to about 90% total amount of one or more agents by total weight of the composition. In some embodiments, one or more of these agents may be added to improve the performance, efficacy, safety, shelf-life and/or other property of the muscarinic antagonist composition of the present disclosure. In some embodiments, additives may be biocompatible, without being harsh, abrasive, and/or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid may be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some embodiments, pharmaceutical compositions disclosed herein may comprise about 5% to about 99%, about 10%, about 95%, or about 15% to about 90% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some embodiments, pharmaceutical compositions disclosed herein may comprise about 5% to about 99%, about 10%, about 95%, or about 15% to about 90% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some embodiments, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition. In some embodiments, pharmaceutical compositions disclosed herein may comprise about 5% to about 99%, about 10%, about 95%, or about 15% to about 90% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In some embodiments, pharmaceutical compositions disclosed herein may comprise about 5% to about 99%, about 10%, about 95%, or about 15% to about 90% total amount of one or more buffering agents by total weight of the composition.

In some embodiments, the amount of one or more buffering agents may depend on the desired pH level of a composition. In some embodiments, pharmaceutical compositions disclosed herein may have a pH of about 6 to about 9. In some embodiments, pharmaceutical compositions disclosed herein may have a pH greater than about 8, greater than about 7.5, greater than about 7, greater than about 6.5, or greater than about 6.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some embodiments, any pharmaceutically acceptable preservative can be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition. In some embodiments, pharmaceutical compositions disclosed herein may comprise about 5% to about 99%, about 10%, about 95%, or about 15% to about 90% total amount of one or more preservatives by total weight of the composition.

In certain embodiments, pharmaceutical compositions disclosed herein may comprise one or more surface-acting reagents or detergents. In some embodiments, surface-acting reagents or detergents may be synthetic, natural, or semi-synthetic. In some embodiments, compositions disclosed herein may comprise anionic detergents, cationic detergents, zwitterionic detergents, ampholytic detergents, amphoteric detergents, nonionic detergents having a steroid skeleton, or a combination thereof. In some embodiments, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some embodiments, pharmaceutical compositions disclosed herein may comprise about 5% to about 99%, about 10%, about 95%, or about 15% to about 90% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition. In some embodiments, pharmaceutical compositions disclosed herein may comprise about 5% to about 99%, about 10%, about 95%, or about 15% to about 90% total amount of one or more stabilizers by total weight of the composition.

In some embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some embodiments, a vapor pressure depression method is used to calculate the osmolarity of the compositions disclosed herein. In some embodiments, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some embodiments, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some embodiments, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some embodiments, pharmaceutical compositions disclosed herein may comprise about 5% to about 99%, about 10%, about 95%, or about 15% to about 90% total amount of one or more tonicity modifiers by total weight of the composition.

### (ii) Dosage Formulations

In certain embodiments, the present disclosure provides compositions formulated for one or more routes of administration. Suitable routes of administration may, for example, include oral, rectal, transmucosal, transnasal, intestinal, and/or parenteral delivery. In some embodiments, compositions herein formulated can be formulated for parenteral delivery. In some embodiments, compositions herein formulated can be formulated intramuscular, subcutaneous, intramedullary, intravenous, intraperitoneal, and/or intranasal injections.

In certain embodiments, one may administer a composition herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to subject as disclosed herein. In some embodiments, a pharmaceutical composition disclosed herein can administered to human patient. In some embodiments, a pharmaceutical composition disclosed herein can administered to a human patient via at least two administration routes. In some embodiments, the combination of administration routes by be intracerebroventricular injection and intravenous injection; intrathecal injection and intravenous injection; intra-cisterna magna injection and intravenous injection; and/or intra-parenchymal injection and intravenous injection.

In certain embodiments, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain embodiments, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain embodiments, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some embodiments, compositions herein may be suspensions, solutions or emulsions in oily or aqueous vehicles, and/or may contain formulatory agents such as suspending, stabilizing and/or dispersing agents.

In certain embodiments, pharmaceutical compositions herein formulated for parenteral administration may include aqueous solutions of the active preparation (e.g., modulator/inhibitor of Wdr37) in water-soluble form. In some embodiments, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some embodiments, compositions herein comprising aqueous injection suspensions may contain substances which increase the viscosity of the suspension, such as sodium carboxymethyl cellulose, sorbitol, and/or dextran. In some embodiments, compositions herein comprising a suspension may also contain one or more suitable stabilizers and/or agents which increase the solubility of the active ingredients (e.g., modulator/inhibitor of Wdr37) to allow for the preparation of highly concentrated solutions.

In some embodiments, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some embodiments, a therapeutically effective amount means an amount of active ingredients (e.g., modulators and/or inhibitors of Wdr37 disclosed herein) effective to prevent, slow, alleviate or ameliorate symptoms of a disorder (e.g., lymphoproliferative disorders, lymphoid malignancy) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some embodiments, toxicity and therapeutic efficacy of the active ingredients disclosed herein can be determined by standard pharmaceutical procedures in vitro, in cell cultures or experimental animals. In some embodiments, data obtained from these in vitro and cell culture assays and animal studies can be used in formulating a range of dosage for use in a human subject. In some embodiments, a dosage for use herein may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1).

In certain embodiments, dosage amounts and/or dosing intervals may be adjusted individually to brain or blood levels of the active ingredient that are sufficient to induce or suppress the biological effect (minimal effective concentration, MEC). In some embodiments, the MEC for an active ingredient (e.g., a modulator and/or an inhibitor of Wdr37 disclosed herein) may vary for each preparation, but can be estimated from in vitro data. In some embodiments, dosages necessary to achieve the MEC herein may depend on individual characteristics and route of administration. Detection assays can be used to determine plasma concentrations.

In certain embodiments, depending on the severity and responsiveness of the condition to be treated, dosing with compositions herein can be of a single or a plurality of administrations, with course of treatment lasting from several days to several weeks or until cure is effected or diminution of the disease state is achieved.

In certain embodiments, amounts of a composition herein to be administered will be dependent on the subject being treated, the severity of the affliction, the manner of administration, the judgment of the prescribing physician, and the like. In some embodiments, effective doses may be extrapolated from dose-responsive curves derived from in vitro or in vivo test systems.

### III. Methods of Use

The present disclosure provides for methods of treating, attenuating, and preventing lymphoproliferation in a subject in need thereof. The present disclosure also provides for methods of treating, attenuating, and preventing at least one lymphoproliferative disease, at least one lymphoid malignancy, or a combination thereof in a subject in need thereof. In certain embodiments, a method for treating, attenuating, or preventing lymphoproliferation or a method for treating, attenuating, or preventing a lymphoproliferative disease and/or lymphoid malignancy in a subject can include administering to a subject, including a human subject, an effective amount of a modulator and/or inhibitor of Wdr37 as disclosed herein.

In certain embodiments, a subject in need thereof can be having, suspected of having, or at risk of having at least one lymphoproliferative disease, at least one lymphoid malignancy, or any combination thereof. In certain embodiments, a subject in need thereof can have one or more genetic markers for a lymphoproliferative disorder. In some embodiments, a subject in need thereof can have one or more genetic mutations in a STIM protein, a ORAI channel, or any combination thereof. In some embodiments, a subject in need thereof can have a FasIpr mutation. In some embodiments, a subject in need thereof can have Bcl2 overexpression. In some embodiments, a subject in need thereof can have one or more genetic mutations in an endive (en) allele, a chicory (ccy) allele, a radical allele, a profound allele, or any combination and/or physiological equivalent thereof. In some embodiments, a subject in need thereof can have one or more genetic mutations of Wdr37, Pacs1, or both wherein the genetic mutation comprises a dominant negative and/or gain-of-function mutation.

In certain embodiments, a subject in need thereof can be an immunocompromised subject. In some embodiments, a subject in need thereof may have had or will have at least one tissue or organ transplant. In some embodiments, a subject in need thereof may be taking one or more immunosuppressant drugs. Non-limiting examples of immunosuppressant drugs can include tacrolimus, cyclosporine, mycophenolate mofetil, mycophenolate sodium, azathioprine, sirolimus, prednisone, and the like.

A suitable subject includes a human, a livestock animal, a companion animal, a lab animal, ora zoological animal. In some embodiments, the subject may be a rodent, e.g., a mouse, a rat, a guinea pig, etc. In some embodiments, the subject may be a livestock animal. Non-limiting examples of suitable livestock animals may include pigs, cows, horses, goats, sheep, llamas and alpacas. In some embodiments, the subject may be a companion animal. Non-limiting examples of companion animals may include pets such as dogs, cats, rabbits, and birds. In some embodiments, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In certain embodiments, methods of treating, attenuating or preventing lymphoproliferation as disclosed herein can be administered immediately before another therapy for lymphoproliferation. In some embodiments, methods of treating, attenuating or preventing lymphoproliferation as disclosed herein can be administered immediately after another therapy for lymphoproliferation. In some embodiments, methods of treating, attenuating or preventing lymphoproliferation as disclosed herein can be administered simultaneously as another therapy for lymphoproliferation. Non-limiting examples of other another therapies for lymphoproliferation can include chemotherapy, rituximab, obinutuzumab, bortezomib, carfilzomib, azacitidine, decitabine, venetoclax, ibrutinib, idelalisib, sunitinib, dinaciclib, cobimetinib, idasanutlin, oblimersen sodium, sodium butyrate, depsipeptide, fenretinide, flavopiridol, gossypol, ABT-737, ABT-263, GX15-070, HA14-1, Antimycin A, acalabrutinib, zanubrutinib, tirabrutinib, bortezomib, lenalidomide, temsirolimus, or any combination thereof.

### IV. Kits

The present disclosure provides kits for use in treating or alleviating a target disease, such as a lymphoproliferative disease and or lymphoid malignancy as described herein. In some embodiments, kits herein can include instructions for use in accordance with any of the methods described herein. The included instructions can comprise a description of administration of a composition containing a modulator and/or inhibitor of Wdr37 disclosed herein and optionally the second therapeutic agent, to treat, delay the onset, or alleviate a target disease as those described herein. The kit may further include a description of selecting an individual suitable for treatment based on identifying whether that individual has the target disease, e.g., applying the diagnostic method as described herein. In still other embodiments, the instructions can include a description of administering an antibody to an individual at risk of the target disease.

The instructions relating to the use of a composition containing a modulator and/or inhibitor of Wdr37 generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., a lymphoproliferative disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). In some embodiments, at least one active agent in the composition can be a modulator and/or inhibitor of Wdr37 as those described herein.

Kits may optionally provide additional components such as buffers and interpretive information. Normally, the kit includes a container and a label or package insert(s) on or associated with the container. In some embodiments, the present disclosure provides articles of manufacture comprising contents of the kits described above.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1. Pacs1 Was Required for Normal Numbers of Circulating Lymphocytes

Dynamic flux of calcium ions (Ca²⁺) between subcellular compartments is required for cell health. The role of Ca²⁺ homeostasis in the adaptive immune system is appreciated primarily through the effects of store-operated calcium entry (SOCE) on lymphocyte activation. Lymphocytes lacking STIM (stromal interaction molecule) proteins or ORAI channels have defects in proliferation and effector differentiation. Patients harboring mutations in these proteins have a severe combined immunodeficiency (SCID) phenotype. Accordingly, there is a need in the field for a better understanding of the role of subcellular Ca²⁺ homeostasis in the development and maintenance of mature lymphocyte populations.

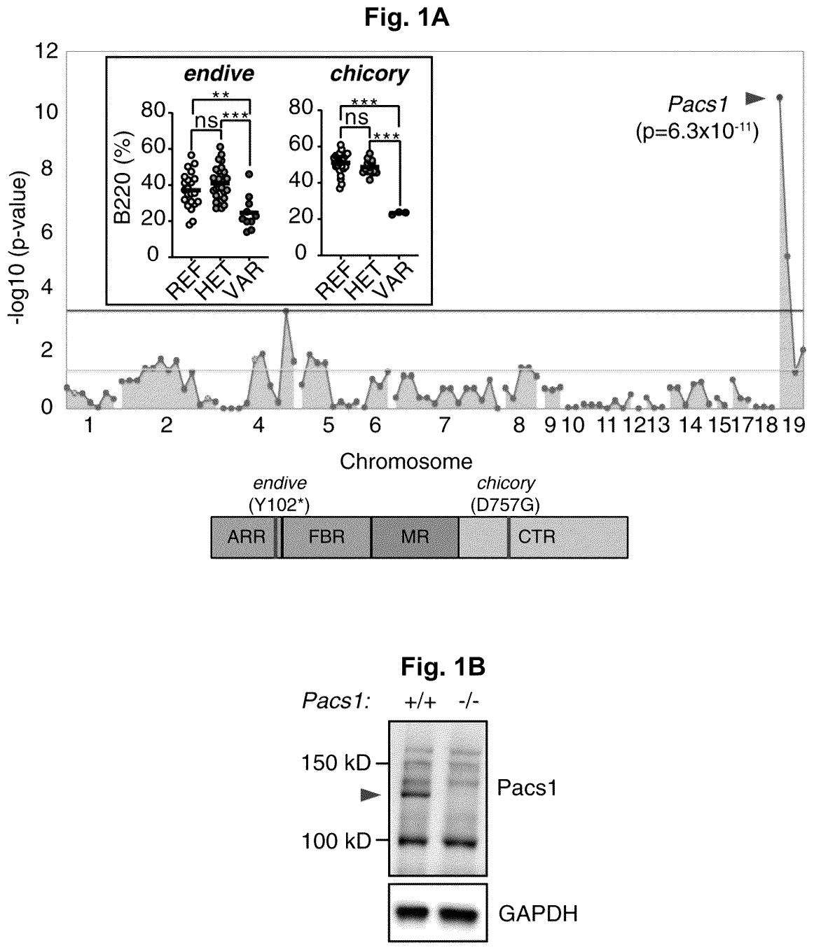

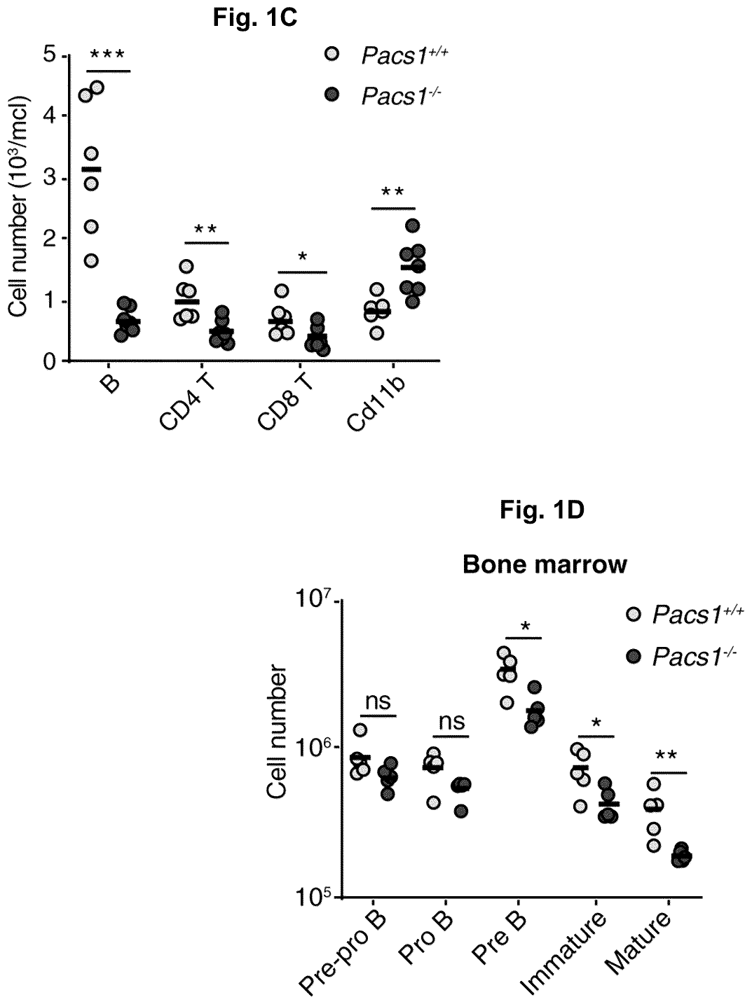

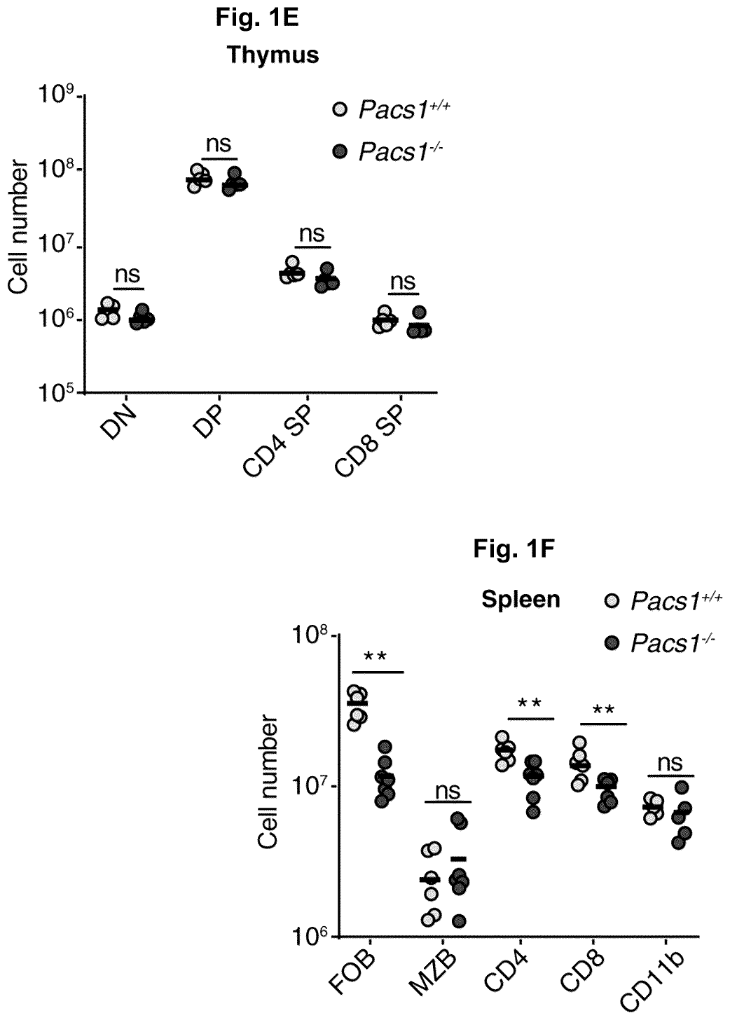

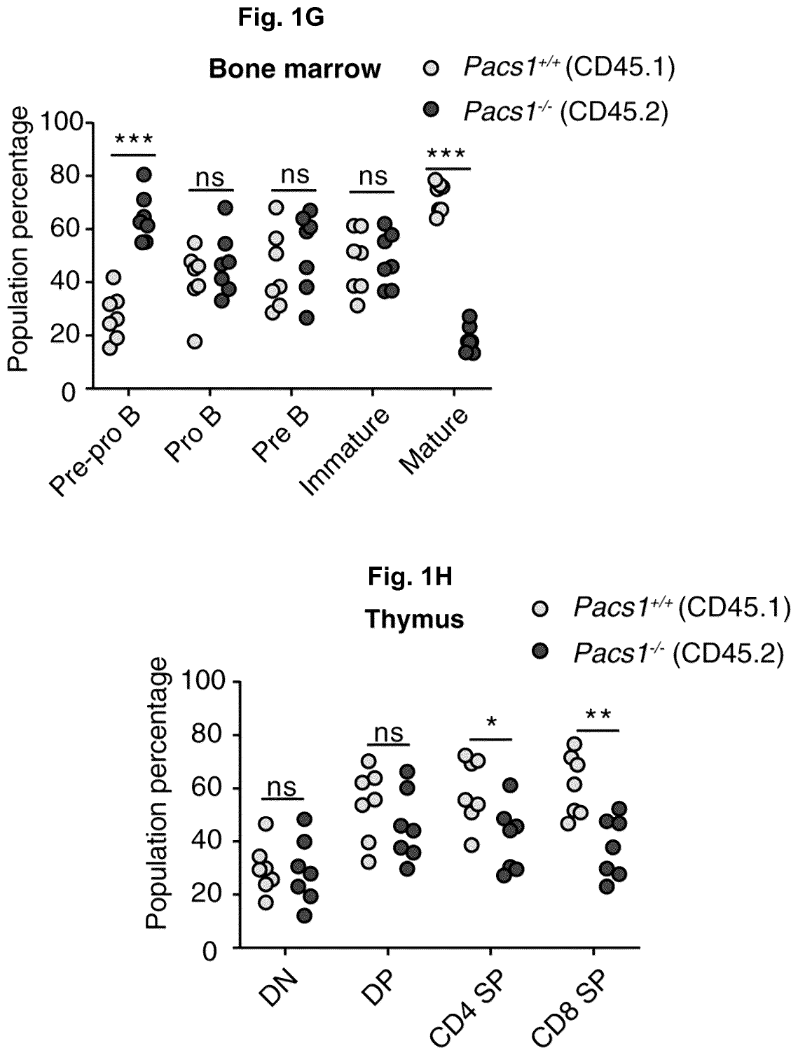

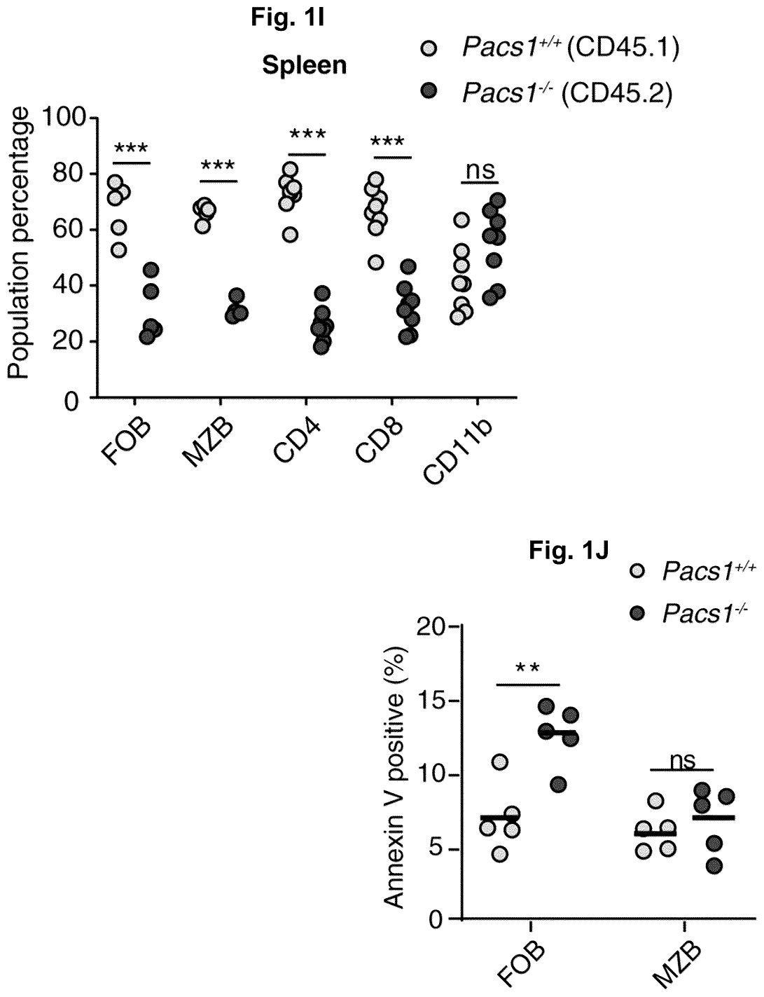

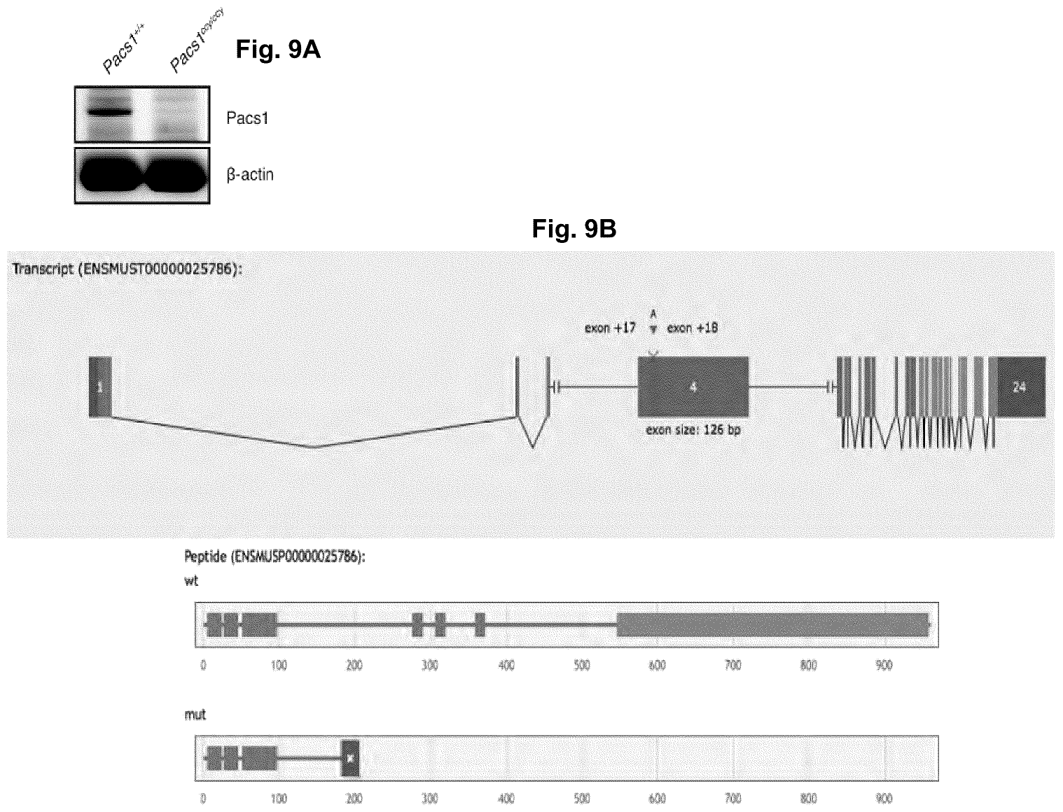

A forward genetic screen was performed in mice mutagenized with N-ethyl-N-nitrosourea (ENU) to identify genes affecting the proportions of circulating immune cell populations according to methods similar to Wang et al., (2015) PNAS 112: E440-9, the disclosure of which is incorporated herein in its entirety. Several mice from two pedigrees showed a diminished proportion of B220+ B cells in the peripheral blood. Automated mapping linked homozygous mutations in both pedigrees to separate mutations in Pacs1 using a recessive model of inheritance. The two alleles were named endive (en) and chicory (ccy). The en mutation was a premature stop codon at Y102 of the Pacs1 protein. The ccy allele was a point mutation (D757G) in the CTR that resulted in complete loss of Pacs1 expression (FIG. 1A and FIG. 9A).

CRISPR/Cas9 editing was used to generate a 1 bp insertion in exon 4 of Pacs1, thereby eliminating protein expression (FIG. 1B and FIG. 9B). Pacs1−/− mice had a deficiency of circulating B cells and CD4 and CD8 T cells, confirming that mutations in Pacs1 were causative of the en and ccy phenotypes (FIG. 1C). Slightly elevated numbers of CD11b+ myeloid cells were observed in the peripheral blood of Pacs1−/− mice.

B and T cell development proceeds through ordered stages in the bone marrow and thymus, respectively. Developing lymphocyte populations were enumerated in primary lymphoid organs to determine how Pacs1 influenced lymphocyte development (FIGS. 1D-1F). Pacs1−/− mice had reduced numbers of B cell progenitors in the bone marrow starting at the pre B stage. This observation was most pronounced in mature recirculating B cells. Pacs1−/− mice showed normal numbers of developing T cell subpopulations in the thymus.

Lymphocyte development was assessed more stringently with competitive bone marrow chimeras (FIGS. 1G-1I). in brief, lethally irradiated Rag2−/− mice were transplanted with 2.5 million cells each of Pacs1+/+; CD45.1 and Pacs1−/−; or CD45.2 bone marrow. The contribution of Pacs1+/+ and Pacs1−/− cells to developing and mature lymphocyte populations was measured in the bone marrow, thymus, and spleen of recipient mice 10 weeks post-transplant based on congenic marker expression. In the bone marrow, chimeric mice showed increased proportions of Pacs1−/− pre-pro B cells, suggestive of a developmental block at this stage. Pacs1−/− cells lost their competitive advantage as they progressed to the pro B, pre B, and immature stages. Pacs1−/− mature recirculating B cells were at a strong competitive disadvantage with respect to Pacs1+/+ cells. In the thymus, Pacs1−/− and Pacs1+/+ developing T cells had equal representation at the double negative and double positive stages. However, CD4 and CD8 Pacs1−/− single positive T cells competed poorly with Pacs1+/+ single positive T cells, revealing a role for Pacs1 in the generation of mature naïve T cells.

In the spleen, Pacs1−/− mice had a 5-fold reduction in follicular B (FOB) cells and normal numbers of marginal zone (MZB) cells. Pacs1−/− mice also had ˜1.5-fold fewer CD4 and ˜2-fold fewer CD8 T cells (FIGS. 1D-1F). Analysis of the spleen in competitive bone marrow chimeras showed that Pacs1 deletion resulted in a competitive defect in both the FOB and MZB cell populations (FIGS. 1G-1I). Additionally, the mild splenic T cell deficiency observed in Pacs1−/− mice was exacerbated under competitive conditions. The myeloid population in the spleen was composed of equal proportions of Pacs1−/− and Pacs1+/+-derived cells.

An increased fraction of Pacs1−/− FOB cells in the spleen was apoptotic based on Annexin V staining, indicating a role for Pacs1 in maintaining this population in the periphery in addition to facilitating their development in the bone marrow. There was no difference in the level of apoptosis between Pacs1^{+/+} and Pacs1^{-/-} MZB cells (FIG. 1J).

### Example 2. Defective ER Ca²⁺ Efflux in Pacs1^{-/-} Lymphocytes After Antigen Receptor Stimulation

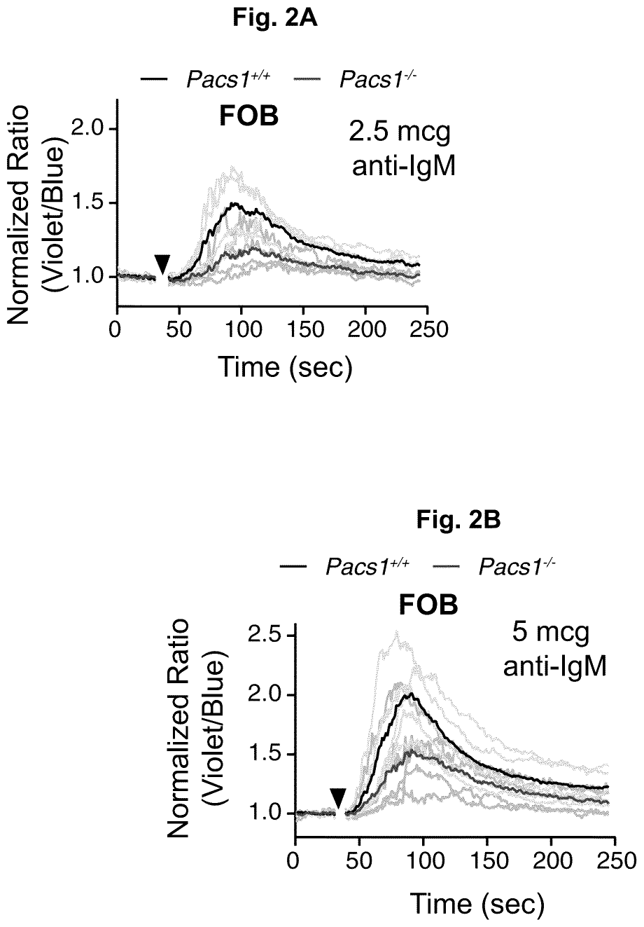

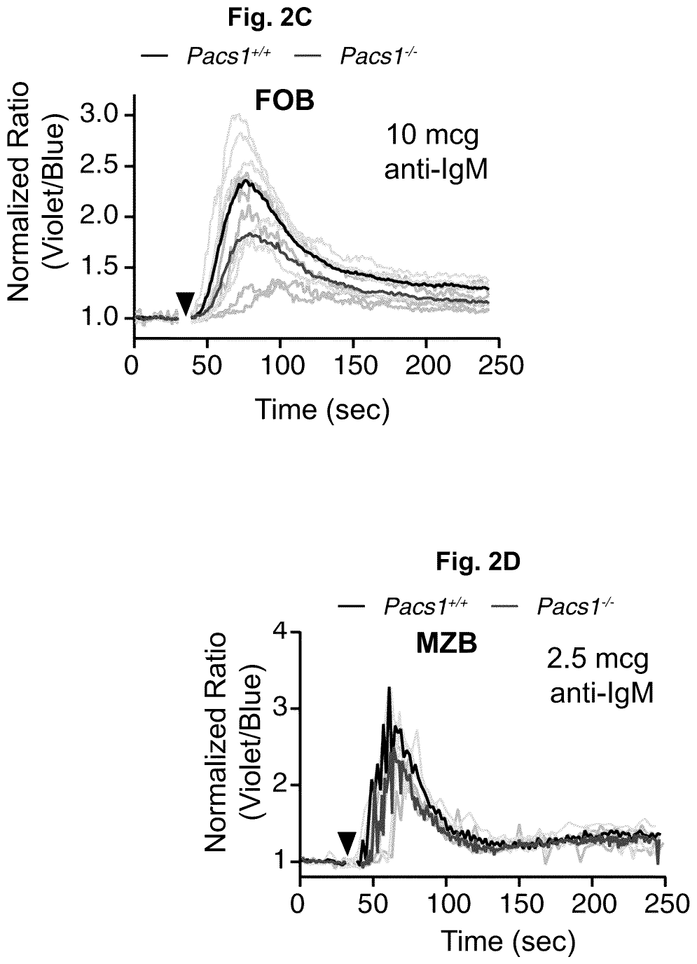

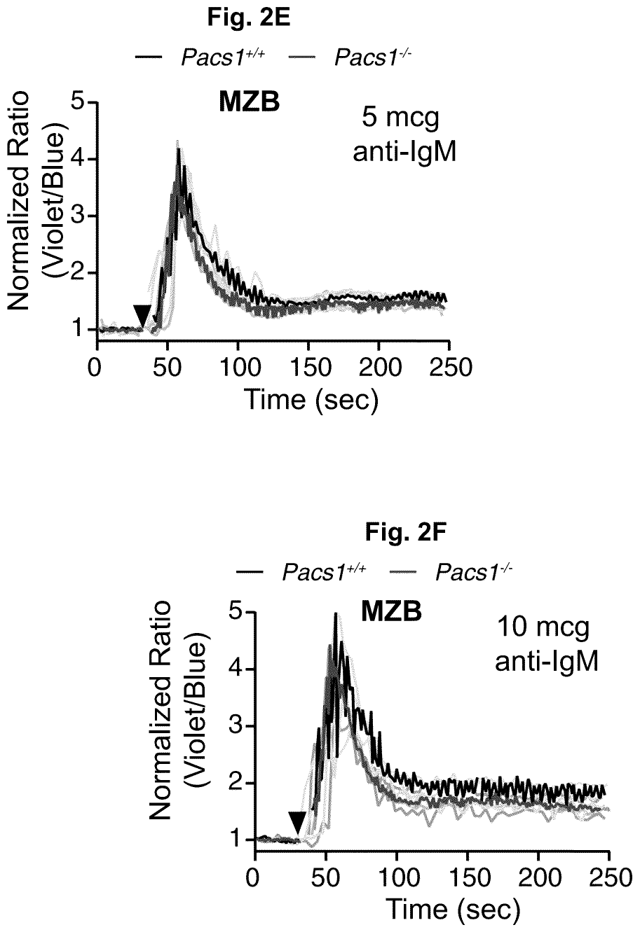

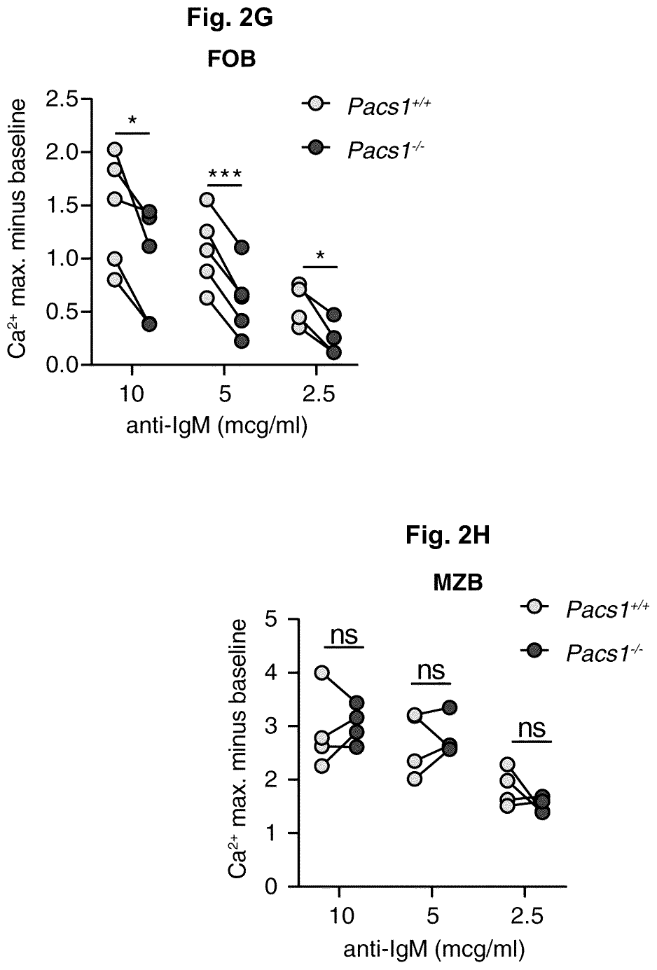

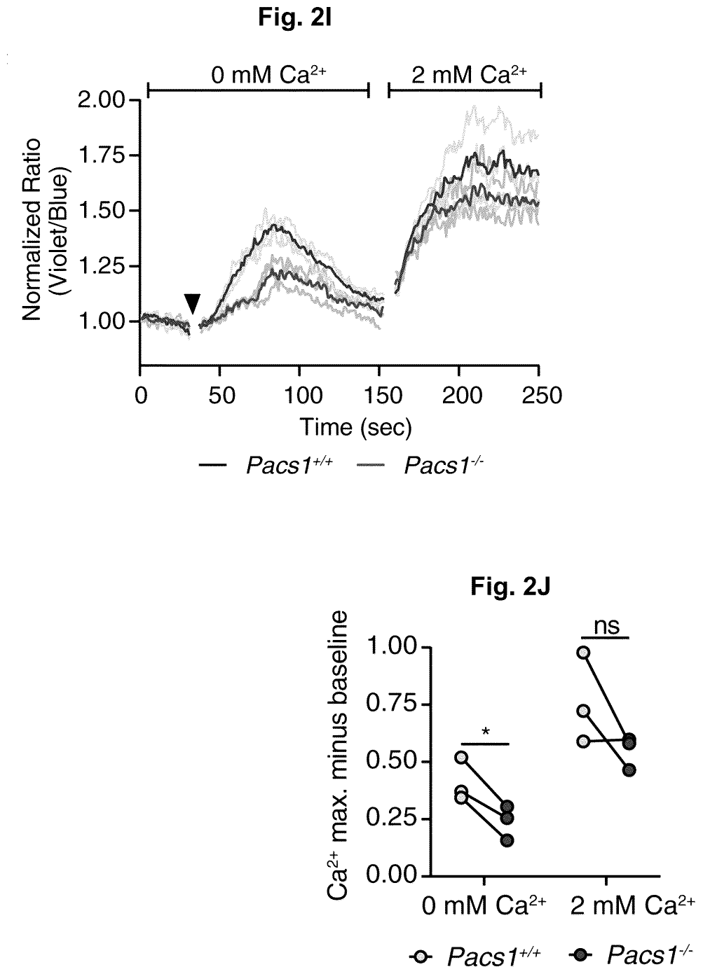

The antigen receptor signaling mechanism is common to both T and B cells and critical to their development and maintenance. To determine if there was a defect in antigen receptor signaling in Pacs1^{-/-} mice contributing to their lymphopenia, splenocytes were loaded with the cytosolic Ca2+ indicator dye Indo-1 and stained for B220, CD21, and CD23 to resolve FOB and MZB cells. Cytosolic Ca2+ flux was measured in response to titrated doses of anti-IgM to stimulate the B cell receptor (BCR) (FIGS. 2A-2H). Pacs1−/− FOB cells showed impaired Ca2+ flux after BCR stimulation at all concentrations of anti-IgM. MZB cells did not show any Ca2+ flux defects compared to Pacs1+/+ controls.

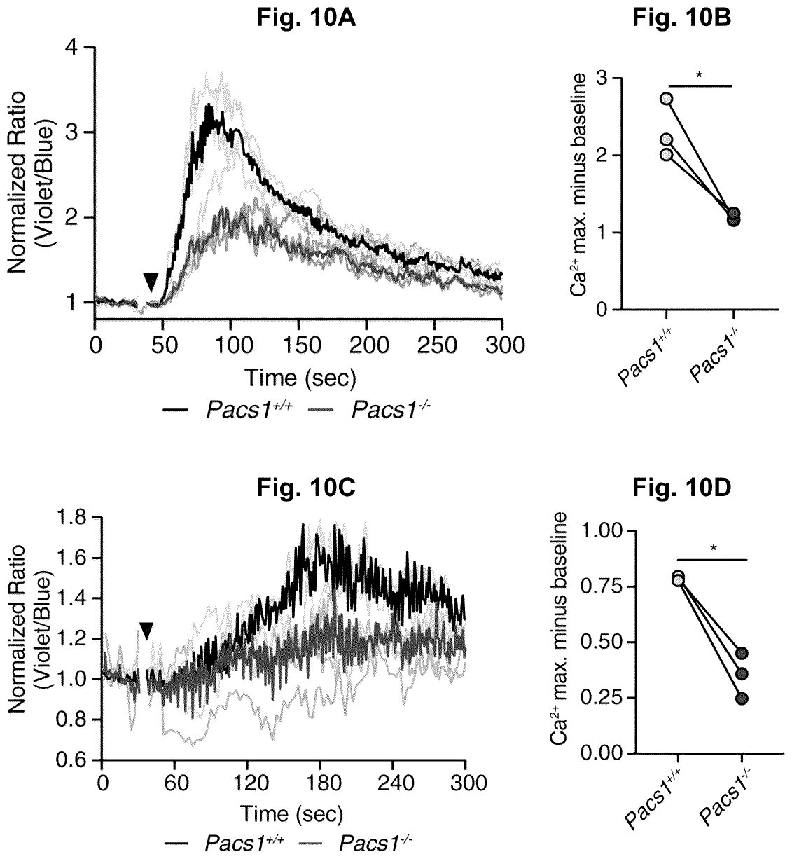

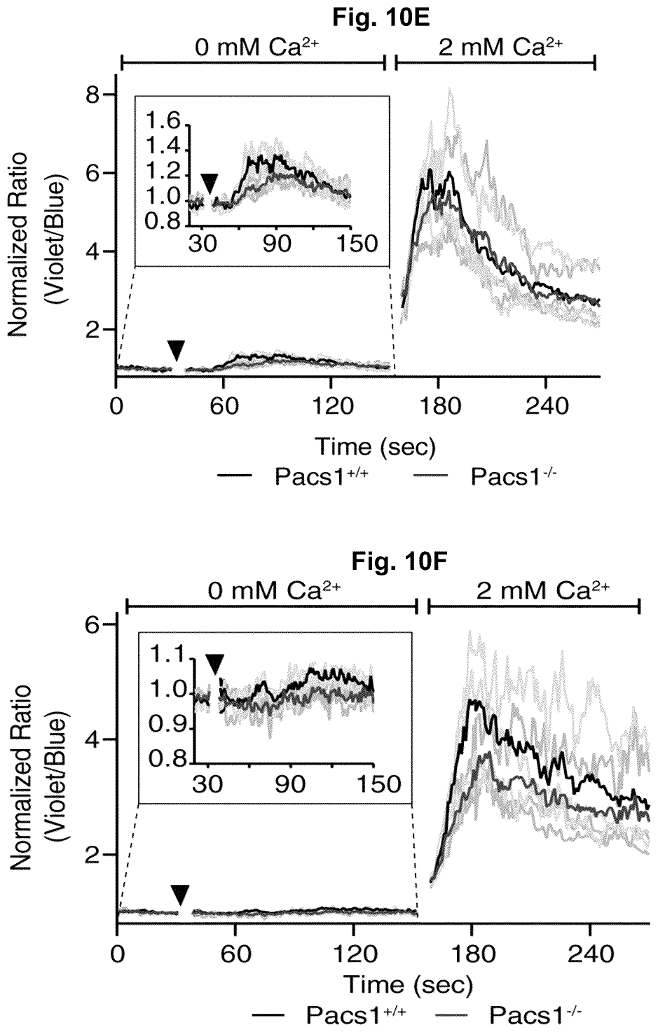

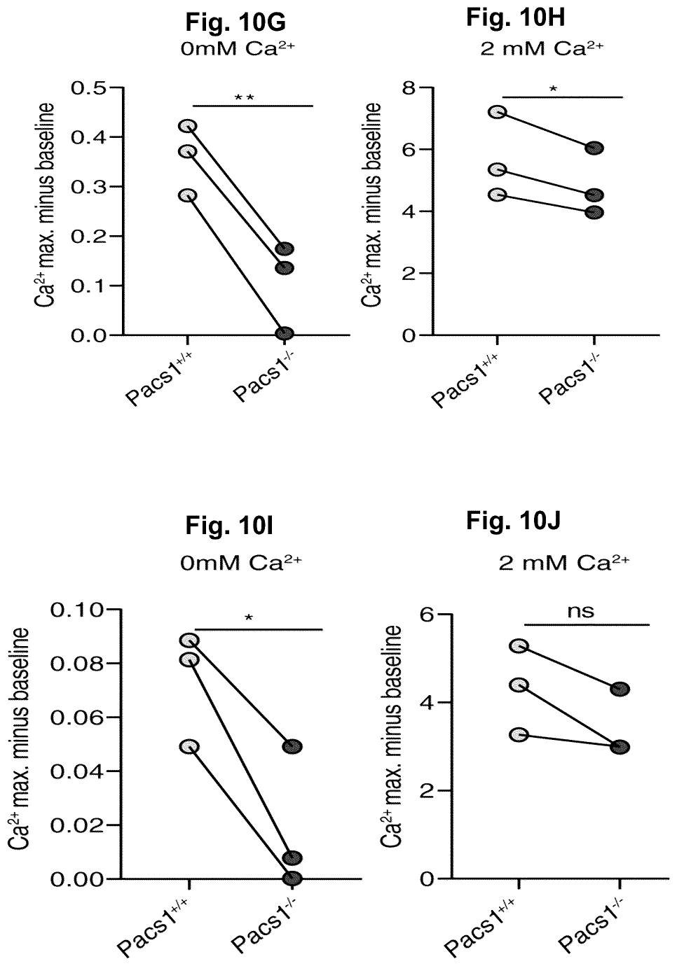

Additionally, Indo-1-loaded splenocytes were stained for CD8 (FIGS. 10A and 10B) and CD4 (FIGS. 10C and 10C) and stimulated with anti-CD3 to crosslink the T cell receptor (TCR) (FIGS. 10A-10J). Both CD8 and CD4 T cells from the Pacs1−/− mice had blunted Ca2+ flux after TCR stimulation.

Next, Indo-1-loaded Pacs1−/− FOB cells were simulated with anti-IgM in Ca2+-free buffer to measure ER Ca2+ efflux. Pacs1−/− FOB cells showed blunted cytoplasmic Ca2+ flux under these conditions (FIGS. 2I and 2J). A defect in ER Ca2+ efflux was observed; however, a significant decrease in SOCE in the Pacs1−/− FOB cells was not observed after adding back 2 mM Ca2+ to the extracellular media.

Splenic CD8 (FIGS. 10E, 10G-10H) and CD4 (FIGS. 10F, 10I-10J) T cells were also stimulated in Ca2+ free media with anti-CD3. A delayed ER Ca2+ release was observed in Pacs1−/− T cells compared to Pacs1+/+ T cells. Like FOB cells, SOCE in T cells was largely intact after adding back 2 mM Ca²⁺. There was a slight reduction in maximal Ca²⁺ flux in CD8 T cells after Ca²⁺ addback and a trend toward reduced maximal Ca²⁺ flux in CD4 T cells that did not reach statistical significance.

### Example 3. Pacs1^{-/-} B Cell Deficiency and Ca²⁺ Flux Phenotypes Were Independent of Antigen Receptor Specificity

To determine if Pacs1 deletion skewed lymphocyte developmental pathways to select for mature populations with the blunted Ca2+ flux phenotype, repertoire skewing in the B cell compartment was controlled for by introducing the B1-8i immunoglobulin heavy chain transgene into the Pacs1−/− background. B cells expressing the B1-8i heavy chain bound 4-hydroxy-3-nitrophenyl hapten (NP) when paired with a lambda light chain.

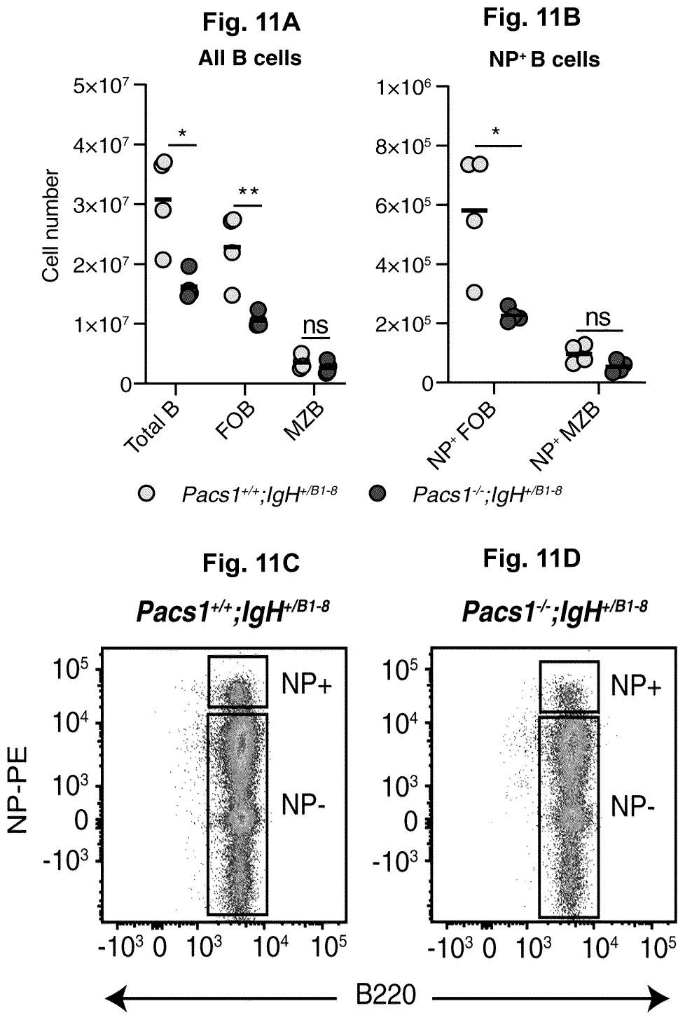

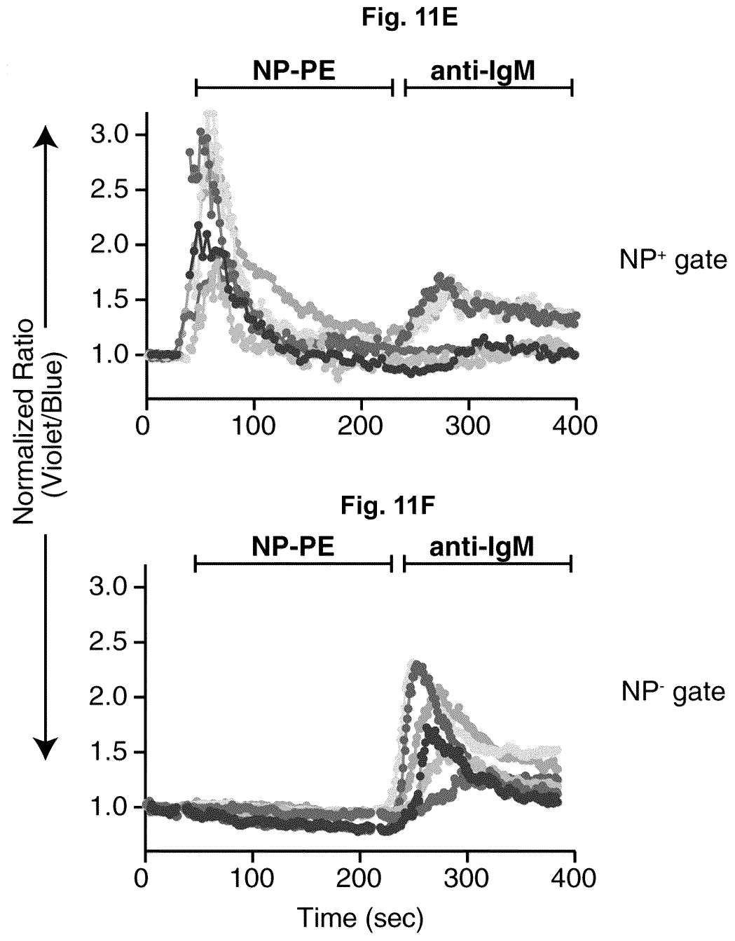

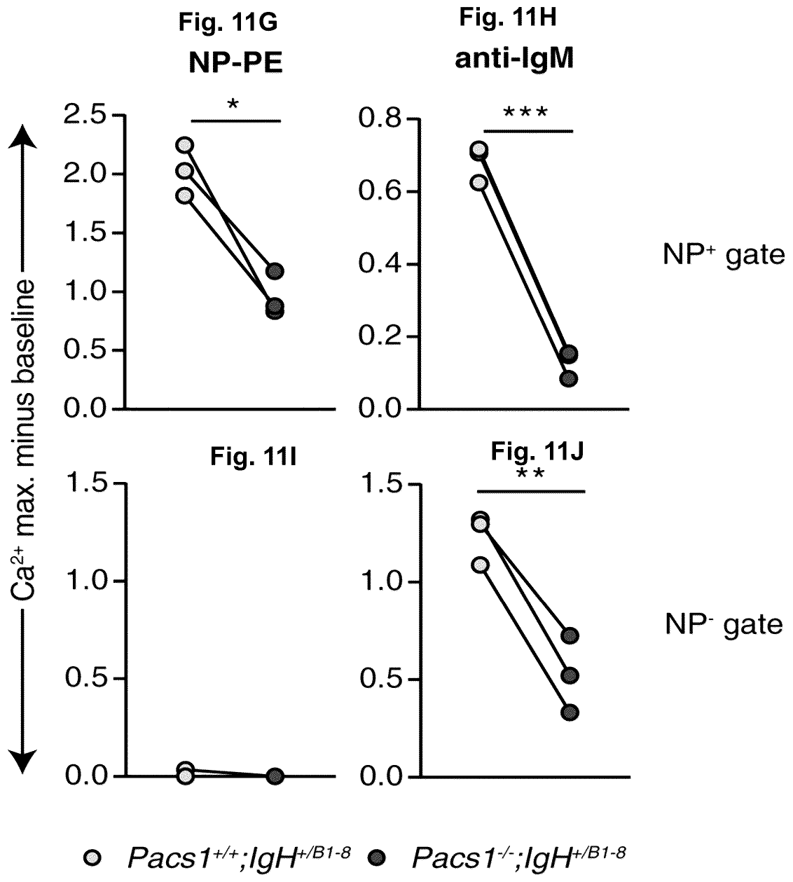

Pacs1−/−; IgHB1-8/+ mice had reduced numbers of total FOB cells in the spleen, while the MZB cell population was preserved (FIGS. 11A-11B). NP-specific B cells were identified by staining with NP conjugated to phycoerthythrin (NP-PE, FIGS. 11C-11D). Within the NP-specific population, there were fewer Pacs1−/− FOB cells than Pacs1+/+ FOB cells. There was no significant difference between the number of NP-specific Pacs1−/− MZB cells and NP-specific Pacs1+/+ MZB cells.

Inducible Ca2+ flux within Indo-1-labeled NP-specific FOB cells was assessed by stimulating with NP-PE. Pacs1−/− NP-specific FOB cells had reduced Ca2+ flux after crosslinking with NP-PE compared to Pacs1+/+ NP-specific FOB cells (FIGS. 11E-11J). Pacs1+/+ NP-specific FOB cells were subsequently stimulated with anti-IgM to induce a second peak in cytosolic Ca2+ flux. Pacs1−/− NP-specific FOB cells were unable to flux cytosolic Ca²⁺ after a second stimulation. The polyclonal FOB cell population (NP-PE negative cells) in both genotypes did not show any cytosolic Ca²⁺ flux after addition of NP-PE. Subsequent addition of anti-IgM showed reduced Ca²⁺ flux amplitude in Pacs1^{-/-} polyclonal FOB cells compared to Pacs1^{+/+} polyclonal FOB cells. Together, these data showed that the FOB cell deficiency and the Ca²⁺ flux defect resulting from Pacs1 deletion were independent of antigen receptor specificity.

### Example 4. Signaling Upstream of ER Ca²⁺ Release Was Intact in Pacs1^{-/-} B Cells

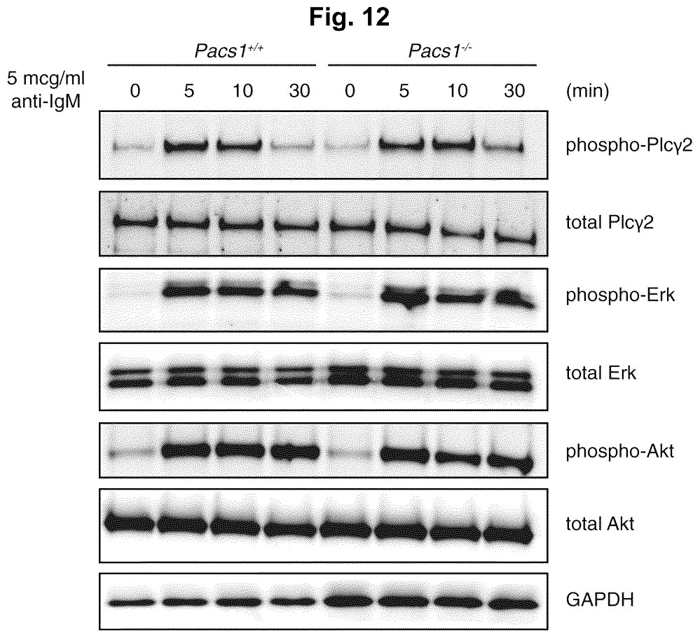

Cytosolic Ca²⁺ flux in lymphocytes is controlled upstream by activated phospholipase C gamma-2 (Plcγ-2). No defect in Plcγ-2 activation in Pacs1^{-/-} B cells was detected after anti-IgM treatment (FIG. 12). The phosphoinositide 3-kinase-protein kinase B/Akt (Pi3K-Akt) and extracellular signal-regulated kinase (Erk) pathways are important for B cell survival and function downstream of antigen receptor stimulation. Data showed that these pathways were also activated normally after BCR crosslinking (FIG. 12). Together, these data indicated that Pacs1 was required for normal Ca²⁺ mobilization in lymphocytes at the level of ER Ca²⁺ release.

### Example 5. Wdr37 Formed a Mutually Stabilizing Complex With Pacs1

Pacs1 is a cytosolic adaptor which facilitates intracellular protein trafficking. To determine if incorrect localization of cargo proteins caused the Pacs1^{-/-} phenotype, co-immunoprecipitation (IP) mass spectrometry was performed on Pacs1-associated protein complexes purified from cell extracts to identify relevant interactor candidates. FLAG-Pacs1 was transfected into 293T cells and affinity purified on anti-FLAG resin. Bead-immobilized FLAG-Pacs1 was incubated with cytosolic extract from purified wild-type murine B cells. The resulting protein complexes were washed and eluted from the anti-FLAG resin and subjected to liquid chromatography tandem mass spectrometry (LC-MS/MS). As a negative control, anti-FLAG beads alone were incubated with B cell extract, washed, eluted, and subjected to LC-MS/MS. 104 proteins were found to be enriched in the FLAG-Pacs1 sample.

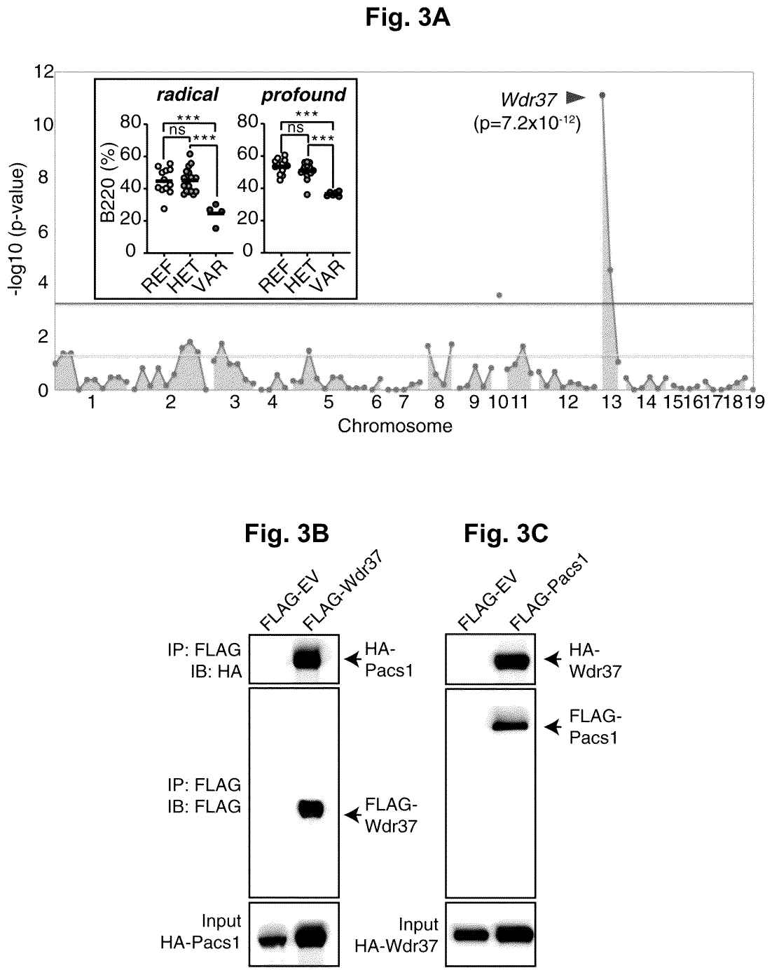

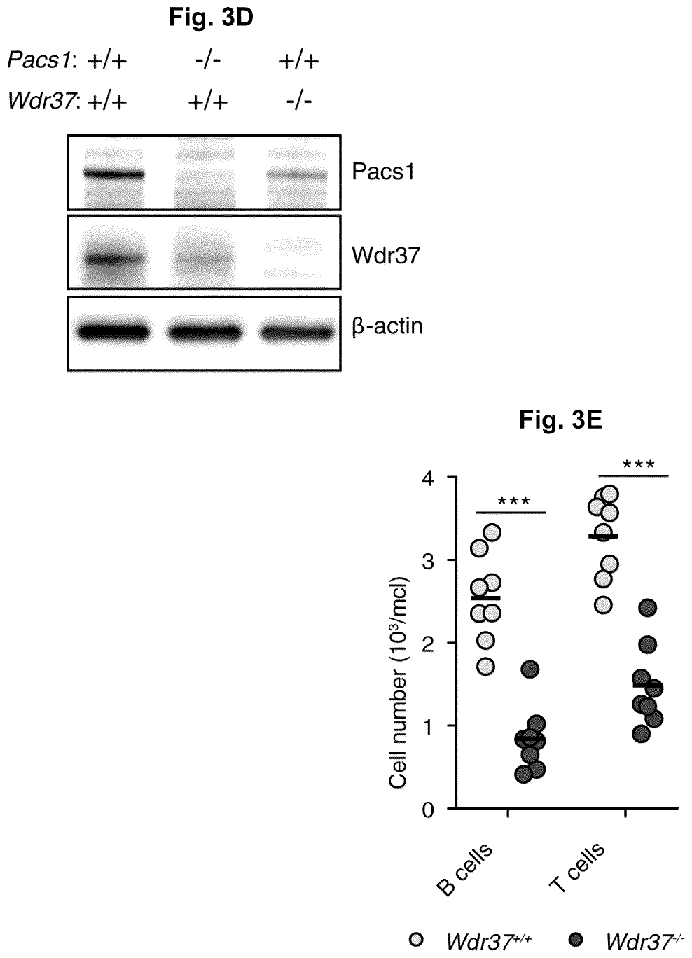

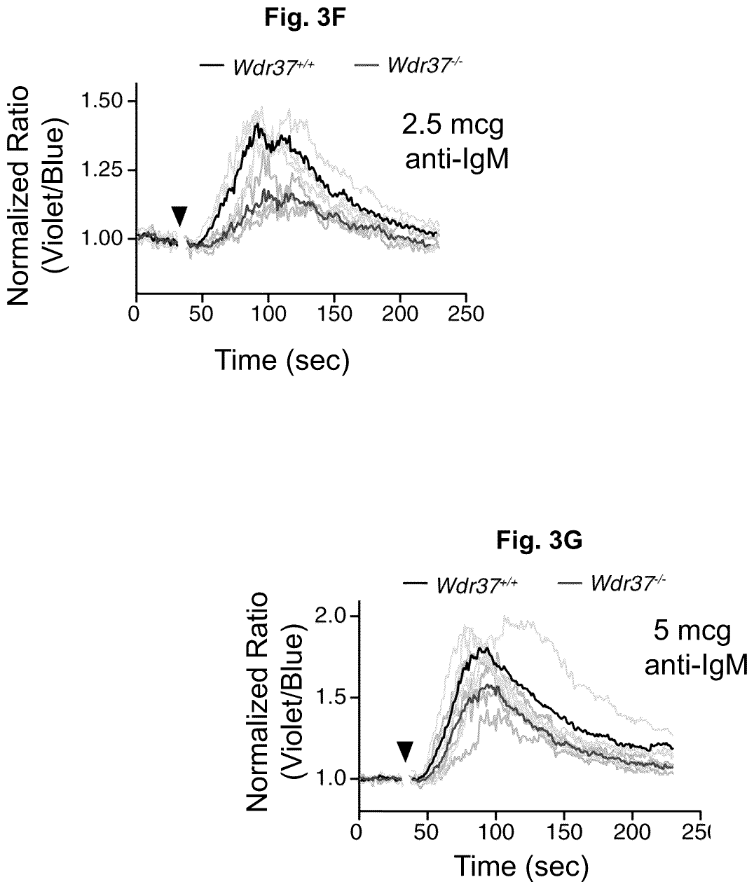

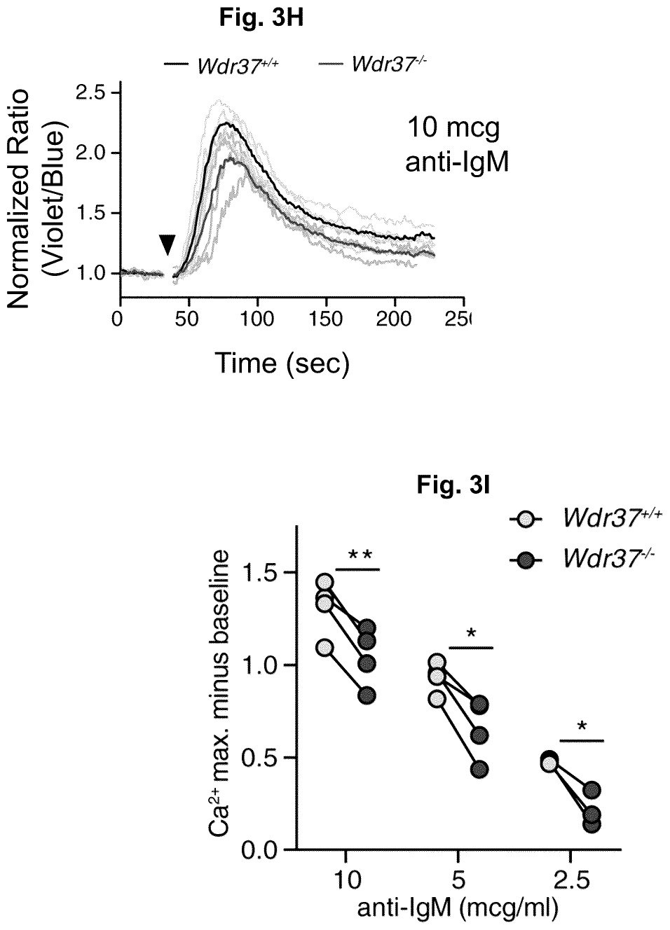

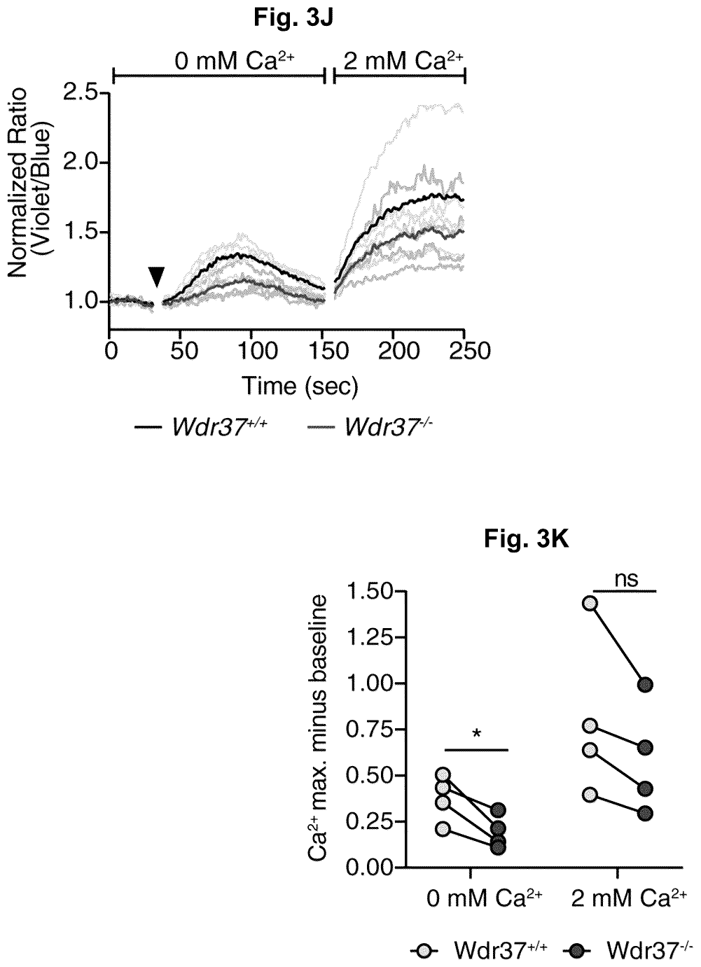

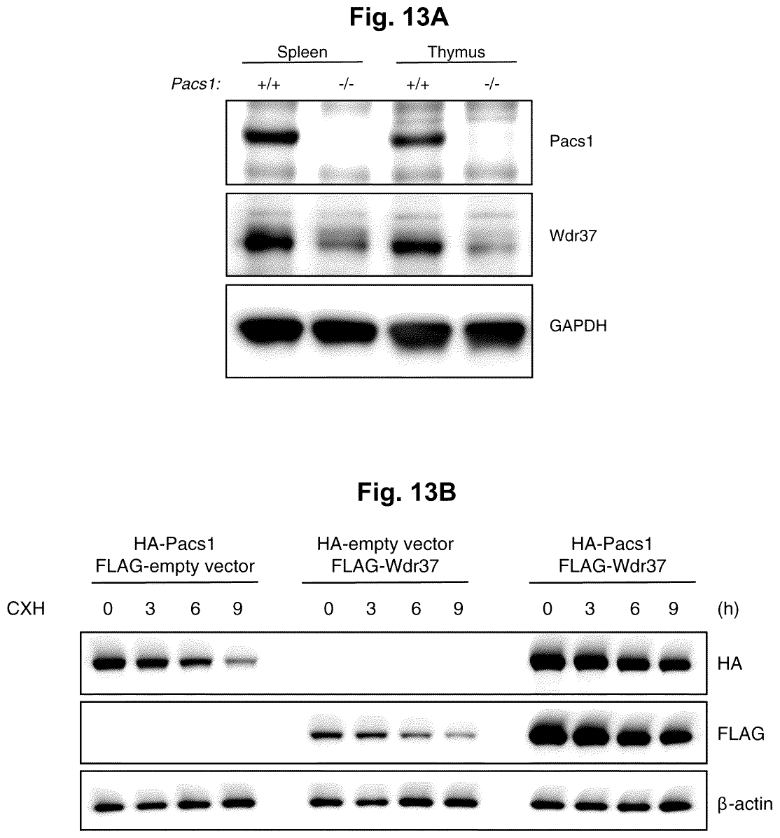

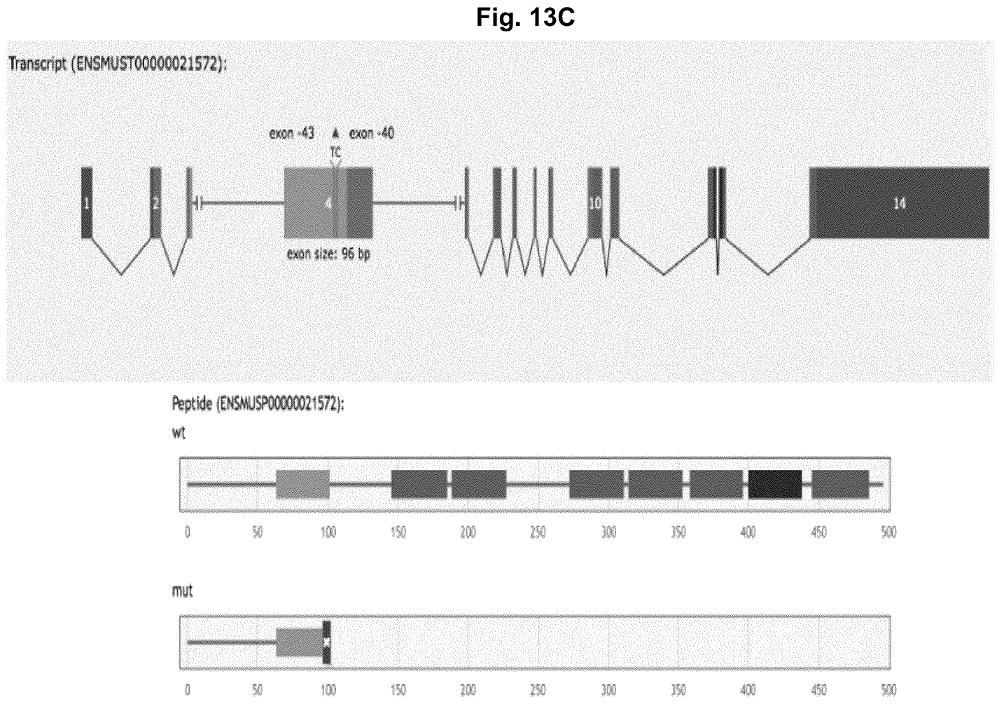

Among the candidate interactors was WD repeat domain protein 37 (Wdr37), within which two ENU-induced mutations linked to a reduction in circulating B cells was identified (FIG. 3A). The initial allele, radical, encoded an early stop codon (L182*). The second allele, profound, was a mutation in a critical splice site which was predicted to be a null allele. In lymphoid tissues, the quantity of Wdr37 protein was markedly reduced in the absence of Pacs1 (FIG. 13A).

To verify physical interaction between Pacs1 and Wdr37, HEK 293T cells were co-transfected with FLAG-tagged Pacs1 (amino acids 171-961) and HA-tagged full-length Wdr37 (FIGS. 3B and 3C). HA-Wdr37 co-immunoprecipitated with FLAG-Pacs1 under these conditions. These proteins also interacted in a reciprocal co-immunoprecipitation experiment using FLAG-Wdr37 as bait and HA-Pacs1 as the target. These data demonstrated Pacs1-Wdr37 complex formation.

CRISPR/Cas9 editing was used to create a frame-shifting 2 bp deletion allele in exon 4 of Wdr37 (FIG. 13C). Peripheral blood cells from these mice lacked detectable Wdr37 and showed reduced levels of Pacs1 expression, suggesting that these proteins stabilized each other in vivo (FIG. 3D). Pacs1 and Wdr37 stability was further evaluated during co-expression using a cycloheximide (CXH) pulse assay in transiently transfected 293T cells. Consistent with a model of mutual stabilization, FLAG-Pacs1 and HA-Wdr37 were expressed at higher levels and decayed more slowly after CXH pulse during co-transfection than when each was expressed separately (FIG. 13B).

Like Pacs1−/− mice, Wdr37−/− mice had reduced absolute numbers of circulating T and B cells (FIG. 3E). Furthermore, Wdr37−/− FOB cells showed blunted cytosolic Ca2+ flux in response to BCR crosslinking (FIGS. 3E-3I). Stimulating these cells in Ca2+ free buffer showed that this phenotype was linked to defective Ca2+ efflux from the ER while SOCE was preserved, as found in Pacs1−/− mice (FIGS. 3J and 3K).

### Example 6. Pacs2−/− Mice Had Normal Proportions of Circulating B Cells

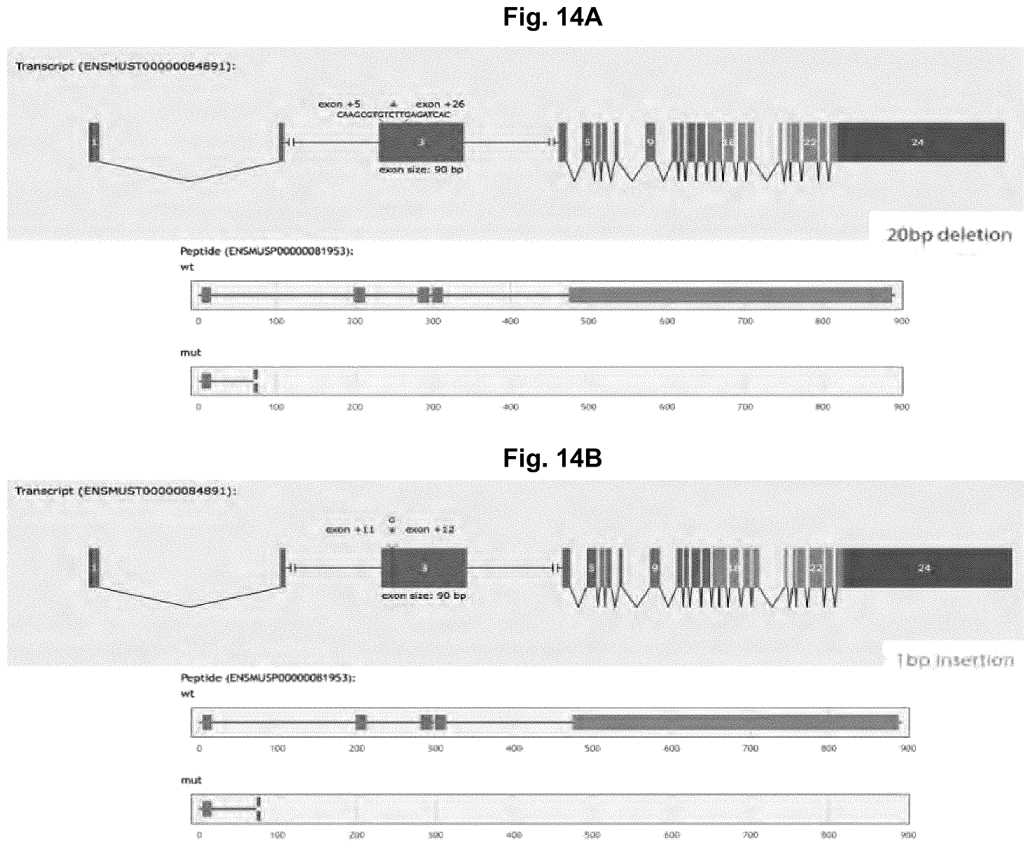

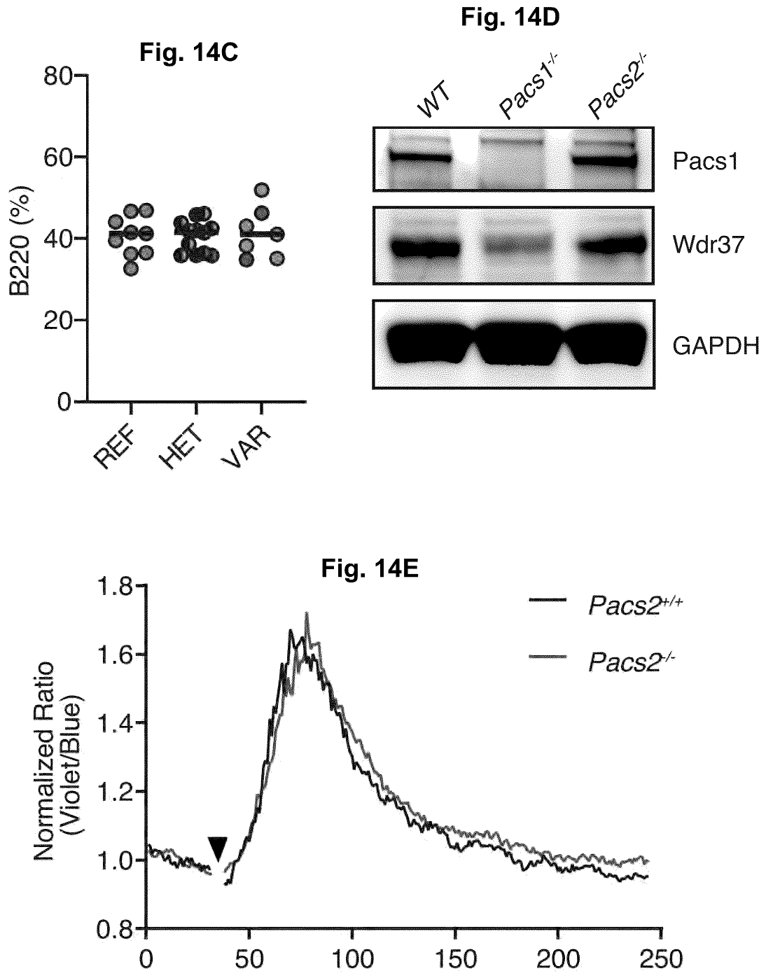

Another candidate interactor identified by mass spectrometry was the Pacs1 homolog Pacs2. Pacs1 and Pacs2 share 54% sequence identity and are generally found in distinct intracellular sorting loops. Knockout alleles of Pacs2 were generated in mice using CRISPR/Cas9 (FIGS. 14A-14B). In contrast to Pacs1−/− mice, no peripheral B cell deficiency was observed in Pacs2−/− mice (FIG. 14C). Additionally, Pacs2−/− FOB cells had normal cytosolic Ca2+ flux after stimulation with anti-IgM (FIG. 14E). Finally, Pacs2 deletion did not reduce stability of Pacs1 or Wdr37 (FIG. 14D). Thus, Pacs1 and Pacs2 had distinct roles in the adaptive immune system, with Pacs1 being uniquely required for maintenance of circulating lymphocyte populations.

### Example 7. Pacs1 Deletion Induced ER Stress, ROS, and Heightened Sensitivity to Oxidative Stress

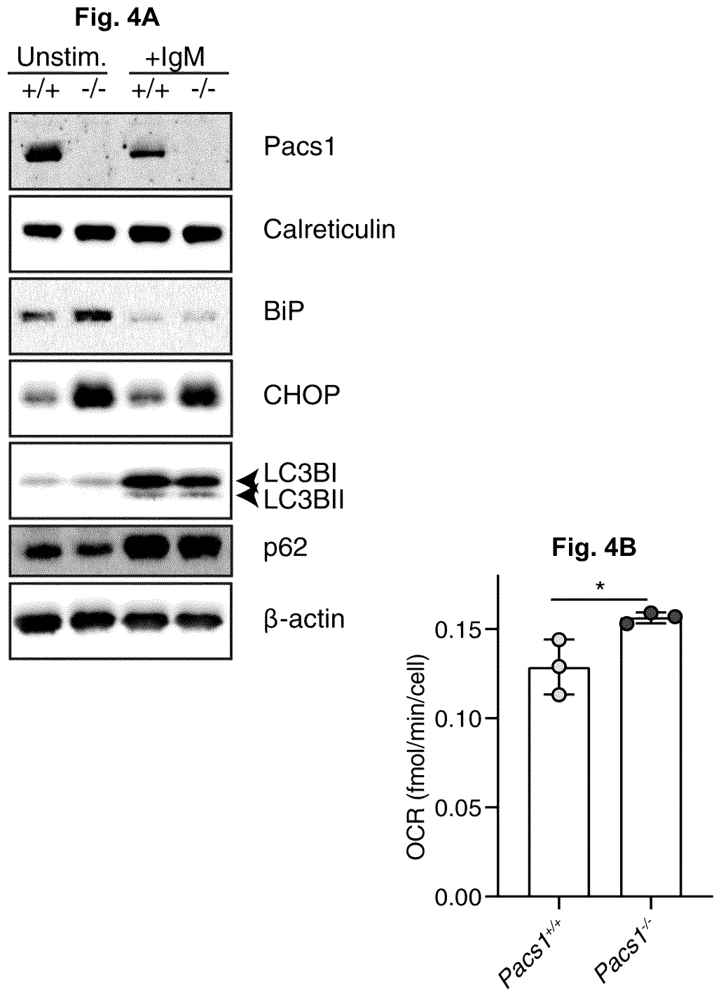

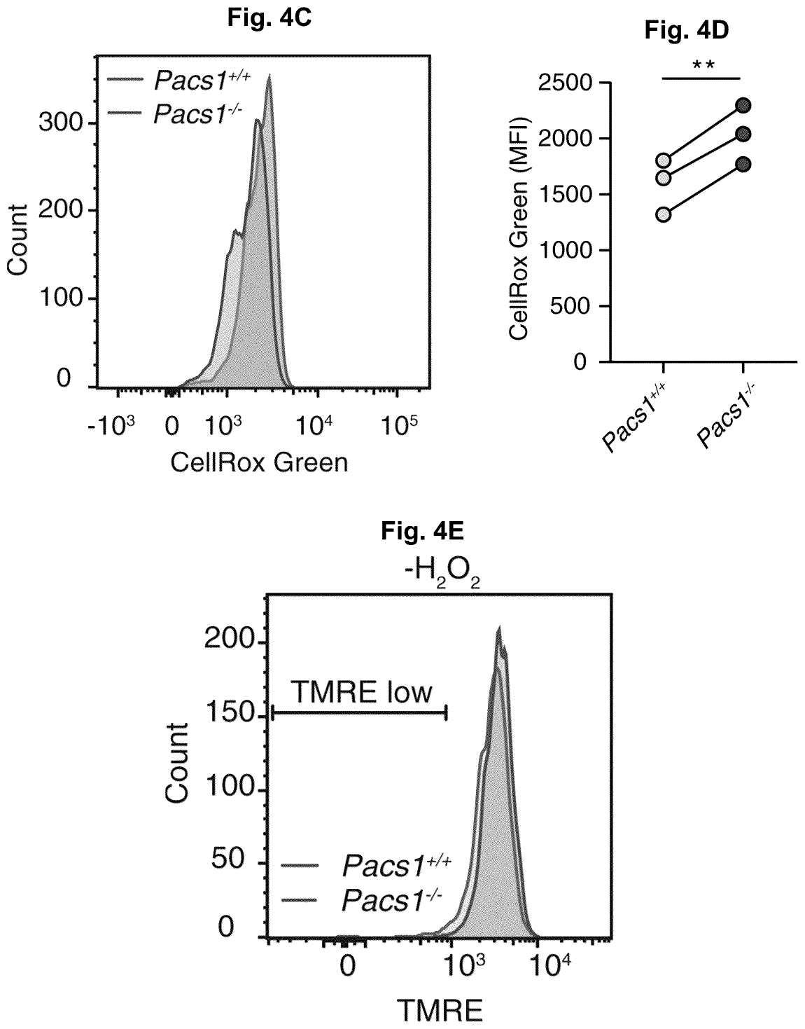

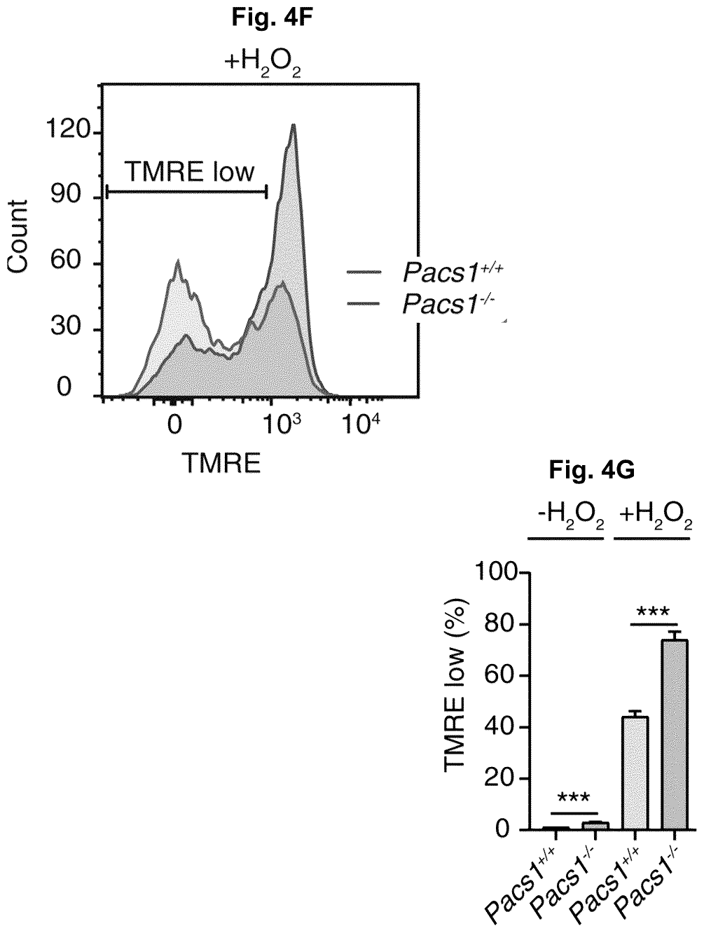

Changes in ER Ca²⁺ homeostasis can cause improper protein folding. To assess whether the defective ER Ca²⁺ efflux in Pacs1^{-/-} lymphocytes might correlate with increased ER stress the amounts of ER stress markers in cells was determined. While Pacs1^{-/-} B cells had ER mass comparable to Pacs1+/+ B cells based on calreticulin expression, they showed substantial upregulation of the ER stress markers Grp78/BiP and CHOP at baseline (FIG. 4A). Stimulation of B cells with 5 mcg/ml anti-IgM overnight reduced BiP expression in both Pacs1+/+ and Pacs1−/− B cells whereas CHOP expression remained elevated in stimulated Pacs1−/− B cells.

ER stress and altered cellular Ca2+ homeostasis can activate or suppress autophagy depending on cellular context. The effect of Pacs1 deletion on autophagy induction was measured in unstimulated splenic B cells and after overnight treatment with 5 mcg/ml anti-IgM (FIG. 4A). In unstimulated Pacs1+/+ or Pacs1−/− B cells, the autophagosome marker LC3B-II was not detected and there was similar basal expression of the autophagy receptor p62. Upon antigen receptor stimulation, similar levels of LC3B-I to LC3B-II conversion between Pacs1+/+ and Pacs1−/− B cells was observed indicating intact autophagy induction. Levels of p62 induction reflected LC3B-II conversion and were independent of Pacs1 genotype.

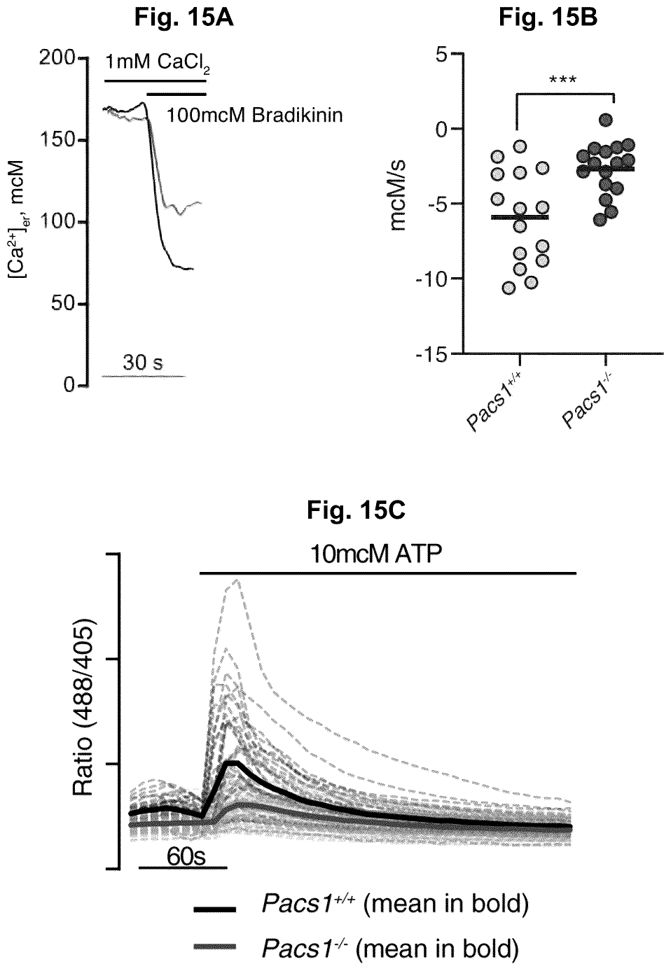

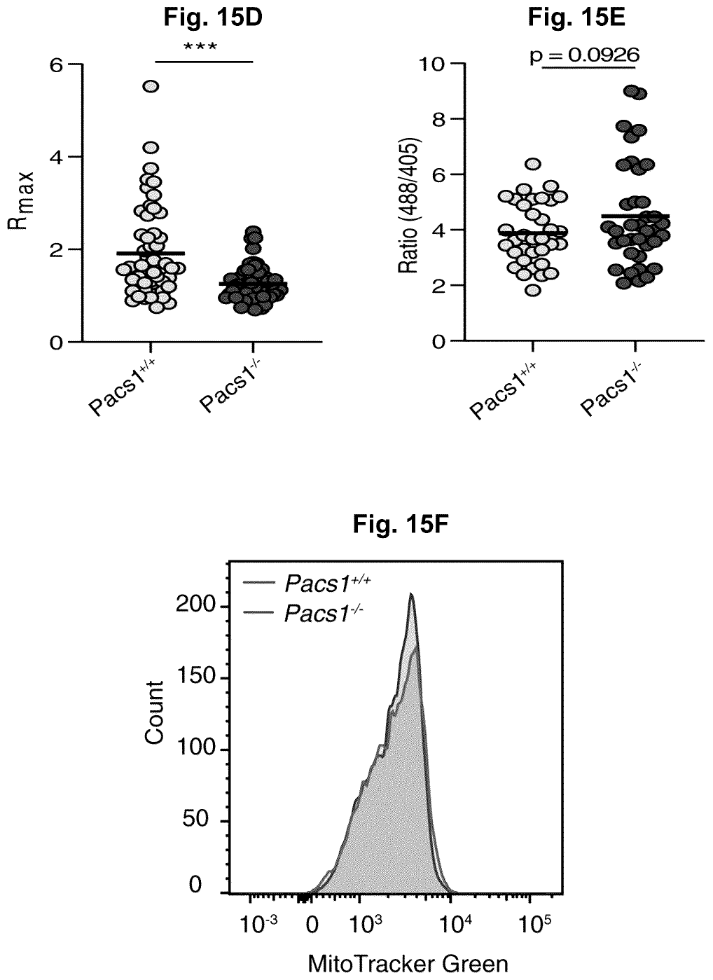

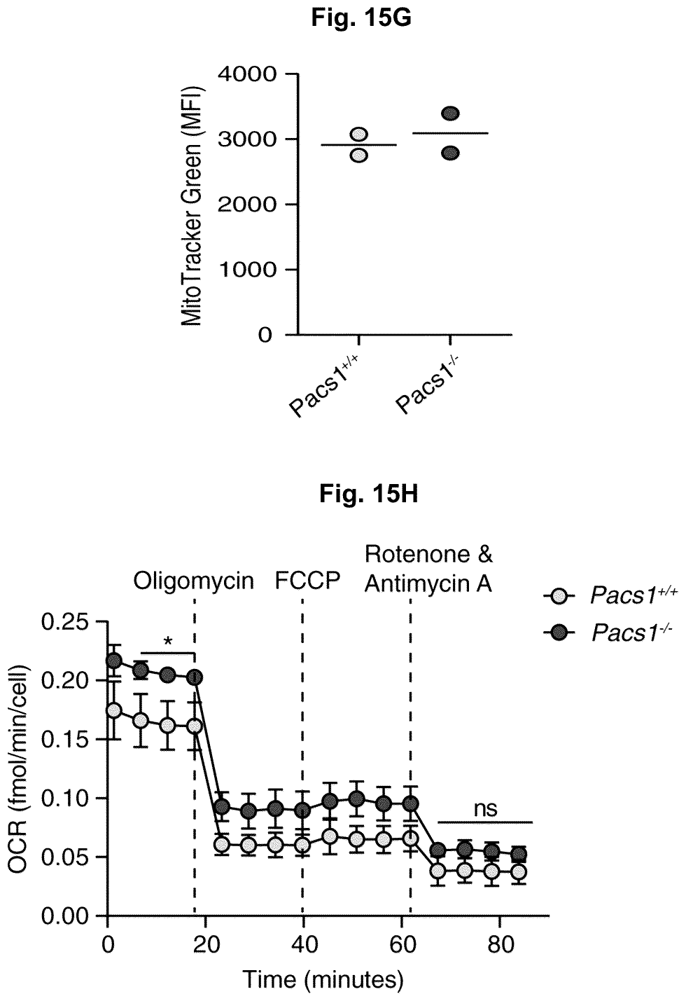

A portion of ER-derived Ca2+ is taken up by the mitochondria where it augments the activity of multiple enzymes involved in oxidative metabolism. To determine how Pacs1 deletion in lymphocytes modulated mitochondrial function, splenic B cells from Pacs1+/+ and Pacs1−/− mice were harvested and oxygen consumption was measured at baseline and after overnight stimulation with 5 mcg/ml anti-IgM (FIG. 4B). Pacs1+/+ and Pacs1−/− B cells contained similar mitochondrial numbers (FIGS. 15F-15G). Oxygen consumption rates (OCR) were measured in purified B cells from Pacs1+/+ and Pacs1−/− mice. It was found that Pacs1−/− B cells had slightly elevated cellular oxygen consumption that increased after antigen receptor stimulation and Pacs1−/− B cells had slightly elevated mitochondrial OCR at baseline (FIG. 15H). Consistent with elevated oxidative metabolism and ER stress, Pacs1−/− B cells also showed increased cellular reactive oxygen species (ROS) based on CelIRox Green staining (FIGS. 4C-4D).

How ER and mitochondrial dysfunction in Pacs1−/− lymphocytes affected their sensitivity to cell death stimuli was examined. Splenocytes from Pacs1^{-/-} mice were stained to identify FOB cells and loaded with TMRE to measure mitochondrial membrane potential (MMP). Cells were then treated with hydrogen peroxide (H₂O₂) to induce oxidative cell death. At baseline, there was a small increase in the number of TMRE low Pacs1^{-/-} FOB cells. After 35 minutes of H₂O₂₋ treatment, ˜75% of Pacs1^{-/-} FOB cells showed loss of MMP compared to ˜40% of Pacs1^{+/+} cells demonstrating increased sensitivity to oxidative stress (FIGS. 4E-4G).

### Example 8. Pacs1^{-/-} B Cells Had Reduced IP3R Expression and ER Ca²⁺ Stores

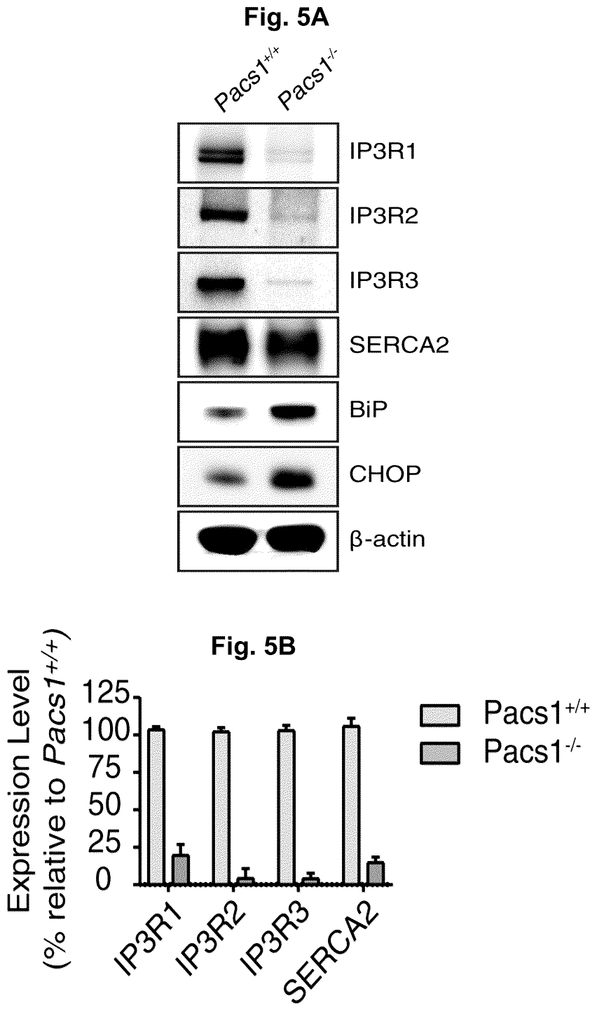

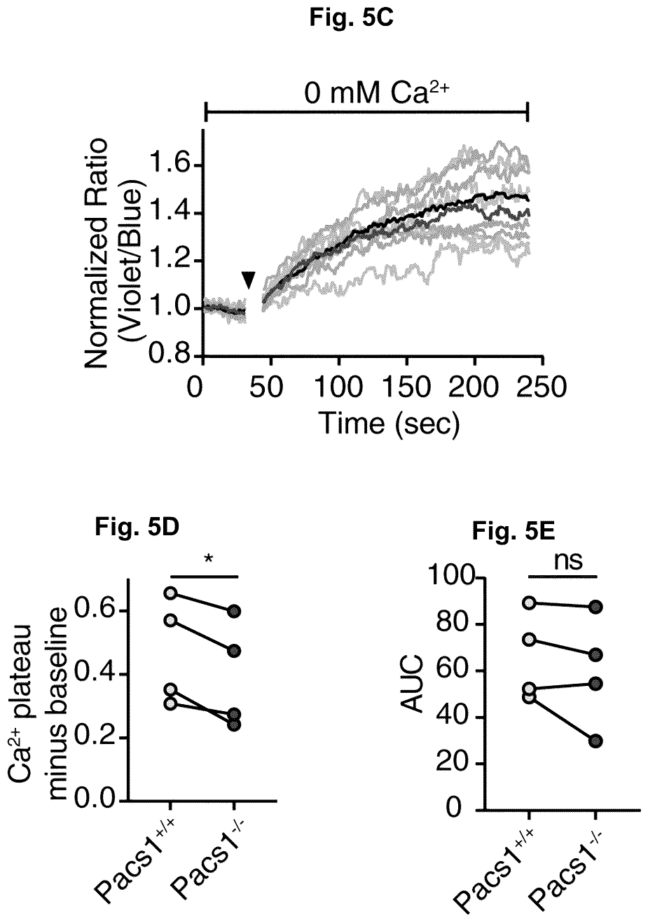

Defective ER Ca²⁺ efflux in Pacs1^{-/-} and Wdr37^{-/-} lymphocytes could be the result of two possible mechanisms: first, ER Ca²⁺ release may be blocked; and second, there may be reduced ER Ca²⁺ content either through diminished storage capacity or chronic leakage. To address these possibilities, protein expression of the three SERCA channel isoforms (SERCA1, SERCA2, and SERCA3) and I P3R isoforms (I P3R1, I P3R2, and IP3R3) was measured in Pacs1+/+ and Pacs1−/− B cells (FIG. 5A). Substantial reduction in the expression of all three IP3R receptor isoforms was found in Pacs1−/− B cells but intact levels of SERCA2 was observed. SERCA1 and SERCA3 protein expression in B cells were not detected.

Surprisingly, it was found that Pacs1-dependent regulation of I P3R expression occurred at the transcriptional level: I P3R1, I P3R2, and I P3R3 mRNA levels were all dramatically lower in Pacs1−/− B cells (FIG. 5B). Also found was reduced transcript levels for SERCA2 in Pacs1−/— B cells, which contrasted with the abundance of SERCA2 protein detected in these cells. Transcripts for SERCA1 and SERCA3 were undetectable in either Pacs1+/+ or Pacs1−/− B cells.

ER Ca2+ stores were next measured in Indo-1-loaded Pacs1−/− FOB cells by stimulating them with the SERCA inhibitor thapsigargin in Ca2+ free media (FIG. 5C). Pacs1−/− FOB cells showed a small but significant decrease in the plateau of cytosolic Ca2+ elicited by thapsigargin compared to Pacs1+/+ FOB cells, indicating diminished ER Ca²⁺ stores (FIG. 5D). Calculating the area under the curve (AUC) of Indo-1 signal revealed no major difference between the two strains and suggested that ER Ca²⁺ stores were largely intact in Pacs1^{-/-} B cells (FIG. 5E). Altogether, these findings suggested that the ER Ca²⁺ efflux defect in Pacs1^{-/-} lymphocytes stems both from decreased expression of I P3Rs and reduced ER Ca²⁺ content.

### Example 9. Pacs1 Deletion Warped ER Ca²⁺ Handling

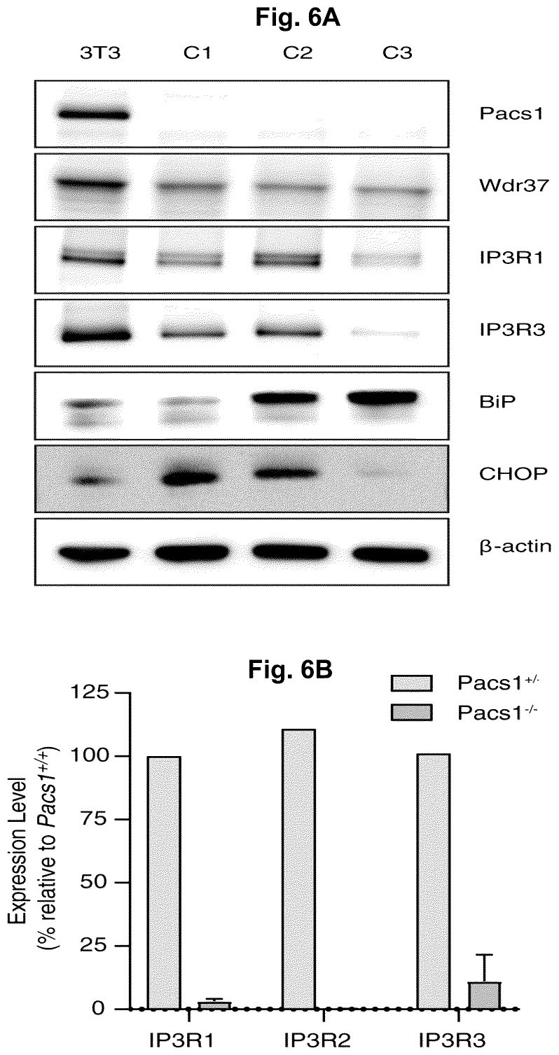

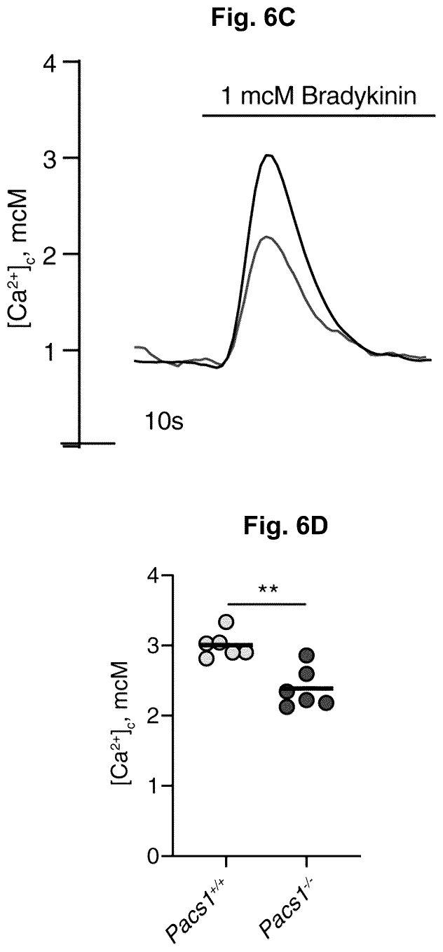

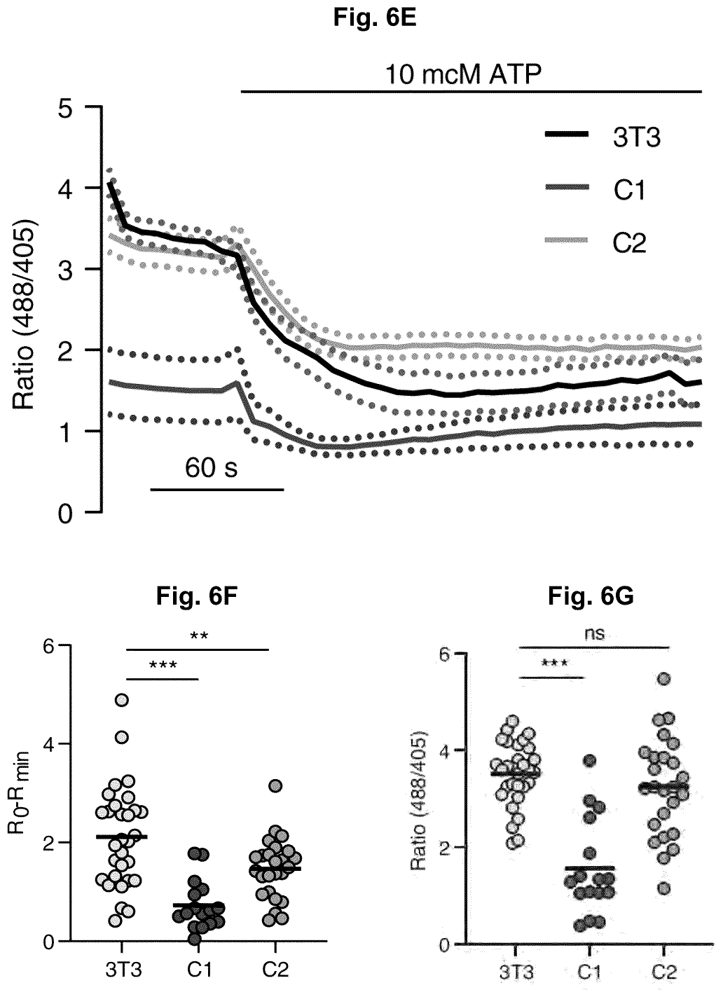

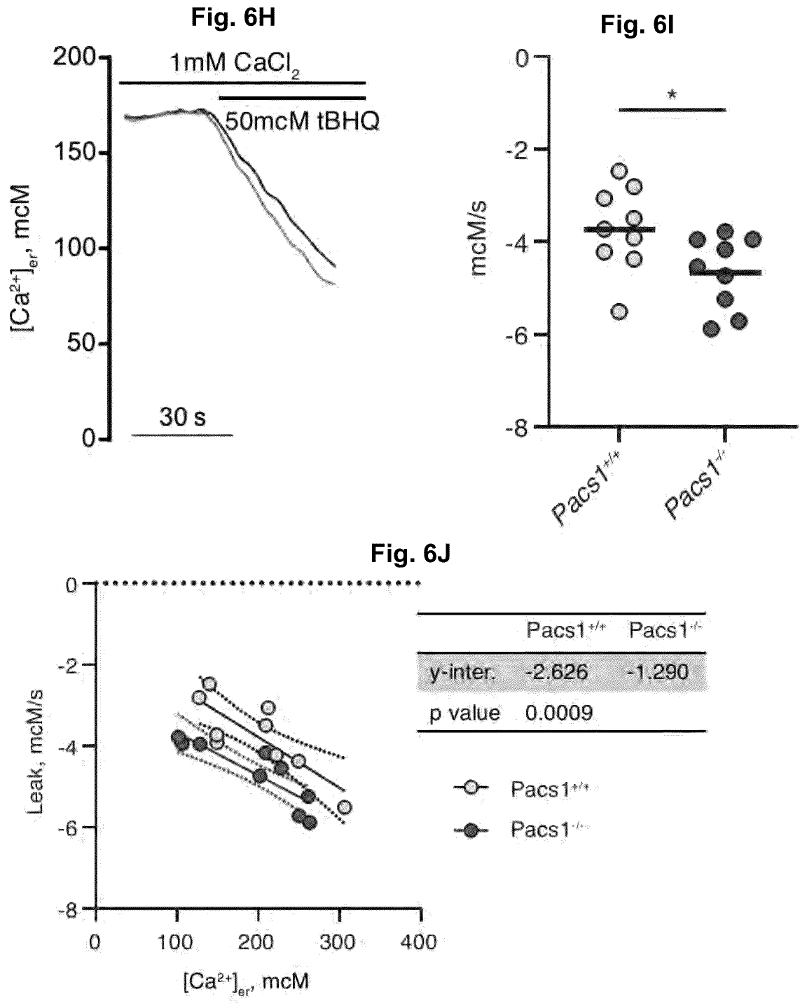

To better determine the role of Pacs1 in Ca²⁺ flux between sub-cellular compartments, Pacs1 was deleted in NIH-3T3 fibroblasts using CRISPR-Cas9 (FIG. 6A). Pacs1^{-/-} 3T3 cells exhibited reduced Wdr37 and IP3R expression and increased ER stress markers. Clonal variation was observed in Pacs1^{-/-} 3T3 cells with respect to the extent of IP3R reduction and BiP and CHOP induction. Additionally, reduced IP3R transcripts were observed in Pacs1−/− 3T3 cells (FIG. 6B). WT and Pacs1−/− 3T3 cells were transfected with a Ca2+ sensitive aequorin construct targeted to the cytosol and it was found that they had blunted Ca2+ flux after IPR3R stimulation with bradykinin (FIGS. 6C and 6D). Therefore, Pacs1−/− 3T3 cells recapitulated several key features observed in Pacs1−/− primary lymphocytes.

Pacs1−/− 3T3 cells were transfected with ER-GCaMP6, a genetically encoded low-affinity ratiometric Ca2+ indicator targeted to the ER. Transfected cells were imaged before and after treatment with ATP to trigger IP3-mediated ER Ca2+ release (FIG. 6E). Pacs1−/− 3T3 cells showed a large reduction in ER Ca2+ release after ATP stimulation which was consistent with reduced IP3R expression in these cells (FIG. 6F). Analysis of pre-stimulation Ca2+ levels also showed select Pacs1−/− 3T3 clones had reduced basal ER Ca2+ content (FIG. 6G). This result, combined with the findings of diminished ER Ca2+ stores in Pacs1−/− FOB cells and heightened ER stress in Pacs1−/− cells, suggested that Pacs1 deletion may also cause chronic ER Ca2+ leakage.

To measure ER Ca2+ leakage with precision, Pacs1+/+ and Pacs1−/− 3T3 cells were transduced with aequorin targeted to the ER (erAEQ). Pacs1−/− 3T3 cells expressing erAEQ showed a strong reduction in ER Ca2+ release after bradykinin stimulation which confirmed results from the ER-CGamP6 Ca2+ reporter (FIGS. 15A and 15B). To assess ER Ca²⁺ leak, cells were treated with the reversible SERCA inhibitor 2,5-t-butylhydroquinone (tBHQ) in Ca²⁺-containing media (FIG. 6H). Pacs1^{-/-} 3T3 cells showed significantly faster ER Ca²⁺ efflux after tBHQ treatment indicating increased basal ER Ca²⁺ leak (FIGS. 6I and 6J). Altogether, ostudies in the 3T3 cell line model demonstrated that Pacs1 deletion affected ER Ca²⁺ handling by blocking Ca²⁺ release through a reduction of IP3R expression and by increasing ER Ca²⁺ leakage.

### Example 10. The Effect of Pacs1 Deletion on Mitochondrial Ca²⁺ Homeostasis

Mitochondrial Ca²⁺ concentration increases upon IP3R-mediated ER Ca²⁺ release. To determine the effects of Pacs1 deletion on mitochondrial Ca²⁺ handling, Pacs1^{+/+} and Pacs1^{-/-} 3T3 cells were infected with MSCV-Mito-Pericam using methods similar to that described in Bohler et al., (2018) Cell Death Dis 9: 286, the disclosure of which is incorporated herein in its entirety. The cells were then stimulated with ATP (FIGS. 15C and 15D). Pacs1^{-/-} 3T3 cells showed substantially reduced maximal mitochondrial Ca²⁺ influx after ATP stimulation which agreed with data herein showing that Pacs1 deletion blunted ER Ca²⁺ release through IP3Rs. When basal mitochondrial Ca²⁺ content was measured in Pacs1^{-/-} and Pacs1^{+/+} 3T3 cells with GCaMP6 targeted to the mitochondria, a significant difference was not detected. (FIG. 15E).

### Example 11. Pacs1^{-/-} B Cells proliferated Normally In Vitro and Pacs1^{-/-} Mice Mounted Normal Humoral Responses After Immunization

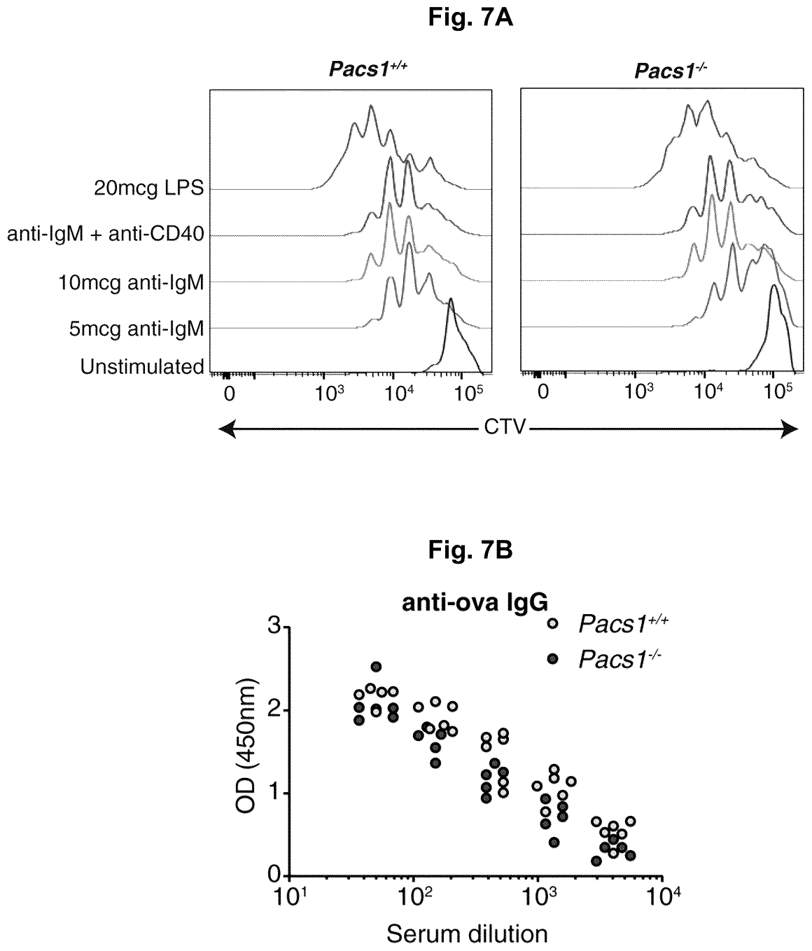

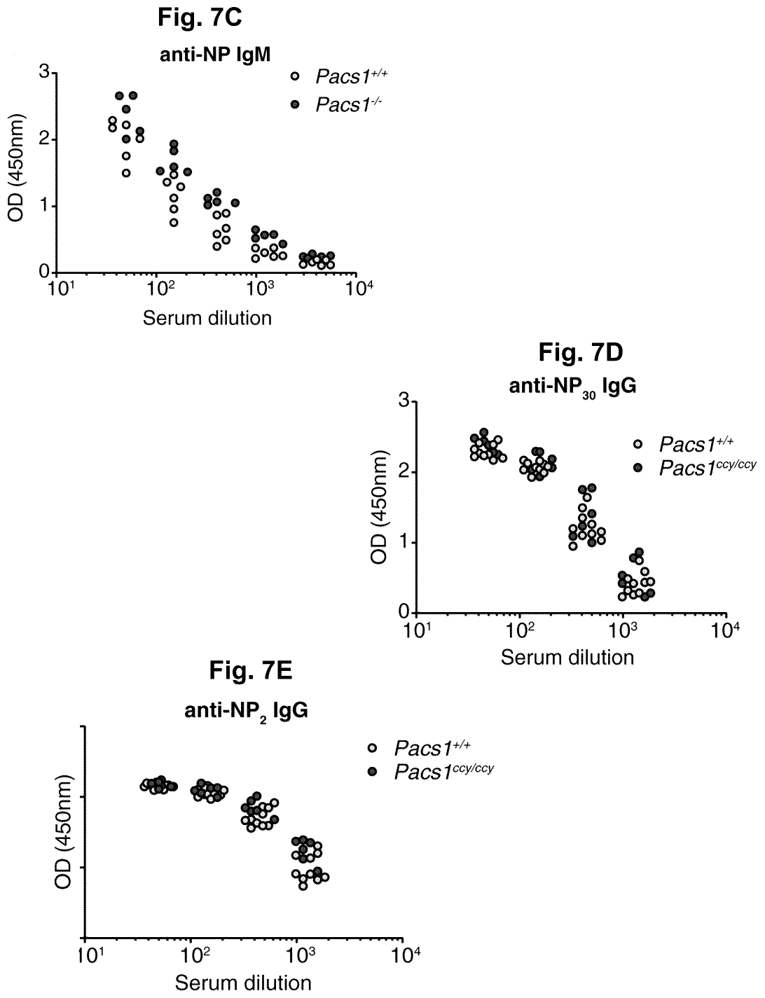

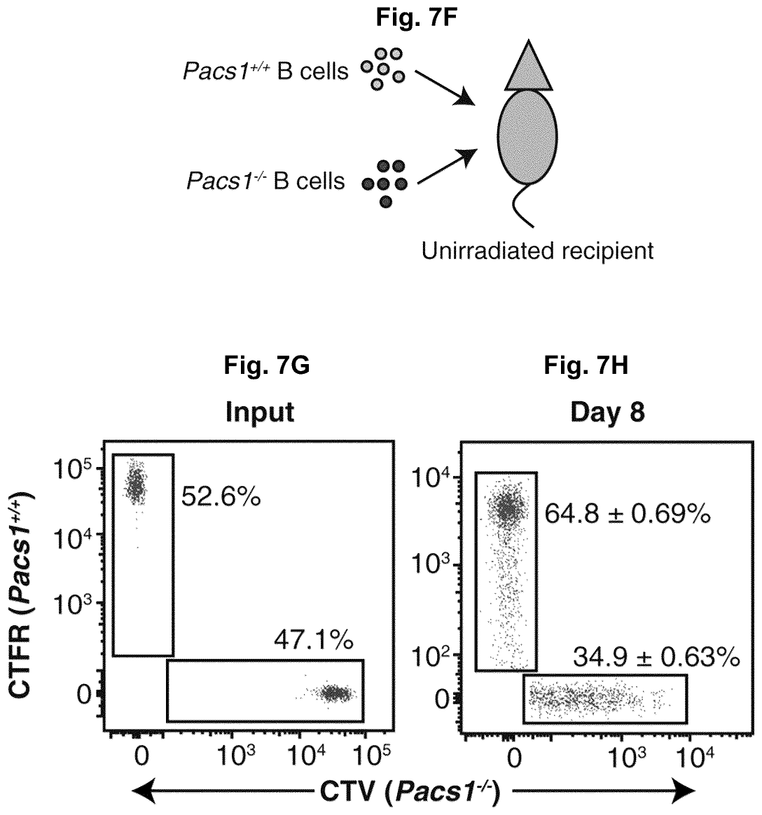

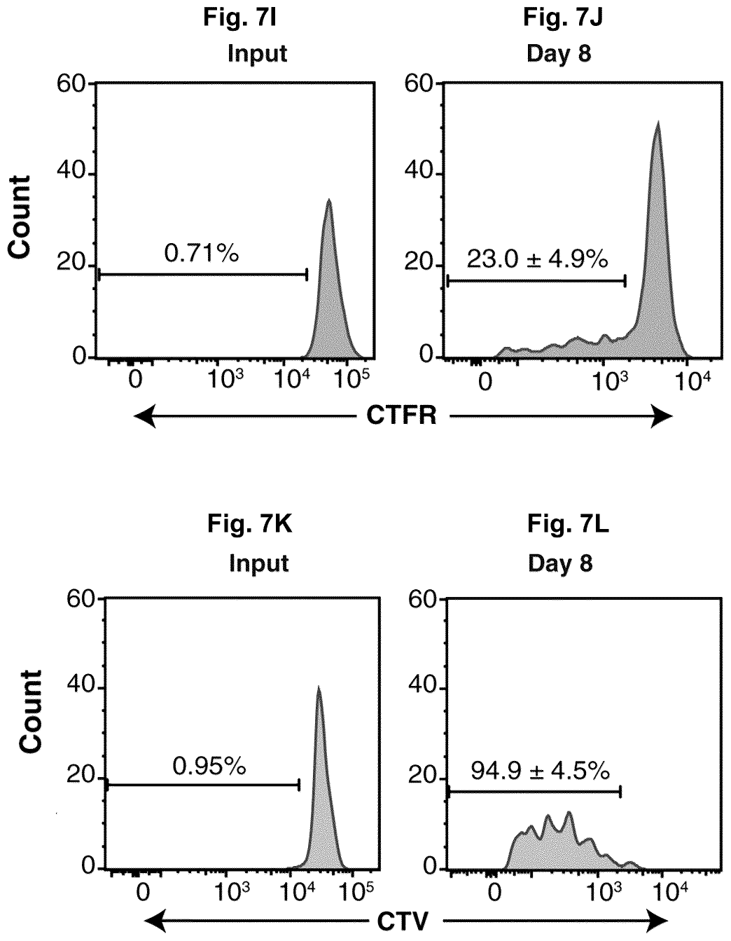

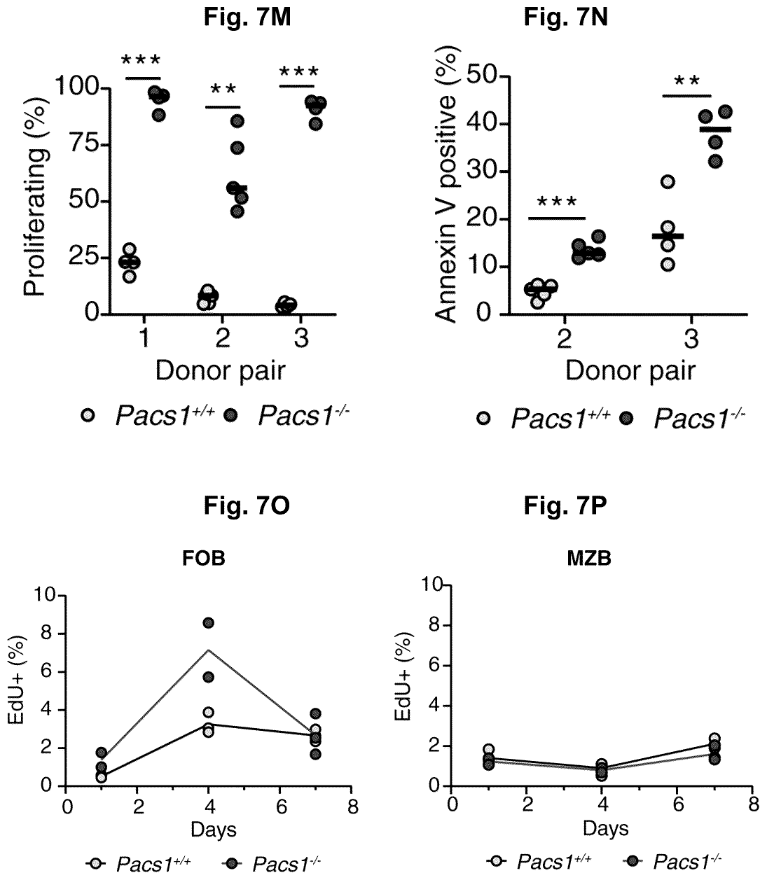

The ramifications of Pacs1 deletion on adaptive immune function was next investigated. Defects in B cell cytosolic Ca²⁺ flux typically result in diminished proliferative responses to antigen receptor stimulation in vitro. B cells were isolated from Pacs1+/+ and Pacs1−/− mice and labeled with CellTrace Violet (CTV) dye. Labeled cells were stimulated with anti-IgM alone, anti-IgM with anti-CD40 to simulate T helper cells, or lipopolysaccharide (LPS). Pacs1−/− B cells showed in vitro proliferative responses comparable to Pacs1+/+ B cells 72 hours after all stimulations (FIG. 7A).

Genetic lesions that block cytosolic Ca2+ flux cause SCID. Pacs1+/+ and Pacs1−/− mice were immunized with ovalbumin precipitated on aluminum salt adjuvant (ova-alum) and NP conjugated to Ficoll (NP-Ficoll) to stimulate T cell-dependent (TD) and T cell-independent (TI) antibody responses, respectively. Pacs1 deletion did not affect either anti-ova IgG titers 14 days after alum-ova immunization or anti-NP IgM titers 7 days after NP-Ficoll immunization (FIGS. 7B and 7C). The importance of Pacs1 for generating high affinity antibodies was assessed the using mice from the chicory (ccy) pedigree. Pacs1^{+/+} and Pacs^{ccy/ccy} mice were immunized with NP-KLH precipitated on alum. IgG titers against NP₃₀-BSA (low affinity IgG) and NP₂-BSA (high-affinity IgG) were identical between the two strains 14 days after immunization (FIGS. 7D and 7E). These data indicated that Pacs1^{-/-} B cells have normal proliferative capacity in vitro and are functional in vivo.

### Example 12. Pacs1^{-/-} B Cells Spontaneously Activated and Died in Lymphocyte Replete Environments

To determine if defective intracellular Ca²⁺ homeostasis combined with increased ER stress and ROS diminished the longevity of Pacs1^{-/-} lymphocytes in vivo, B cells were isolated from the spleens of Pacs1^{+/+} and Pacs1^{-/-} mice and labeled them with CellTrace Far Red (CTFR) and CTV dye, respectively. Labeled B cells were transferred at a 1:1 ratio into non-irradiated CD45.1 recipients (FIGS. 7F-7L). Adoptively transferred B cells were detected in the spleens of recipient mice 8 days post-transfer by staining for CD45.2 and measuring CTFR and CTV fluorescence. In this assay, most transferred B cells should not undergo cell division because there is no stimulus for homeostatic expansion without lymphotoxic pre-treatment of recipient. Accordingly, <25% of adoptively transferred Pacs1+/+ B cells diluted CTFR after adoptive transfer. Strikingly, >95% of adoptively transferred Pacs1−/− B cells spontaneously proliferated by 8 days after transfer (FIGS. 7F-7M). This was accompanied by poor recovery of adoptively transferred Pacs1−/− B cells relative to Pacs1+/+ B cells from the spleens of recipient mice. Higher rates of apoptosis were also observed in adoptively transferred Pacs1−/− B cells, as measured by Annexin V staining (FIG. 7N).

The effects of Pacs1 deletion on B cell turnover under steady state conditions was verified using pulse-chase analysis with the thymidine analog 5-ethynyl-2′-deoxyuridine (EdU). Pacs1+/+ and Pacs1−/− mice were given a single injection of EdU to label actively cycling cells in primary and secondary lymphoid organs (FIGS. 7O and 7P). Splenocytes were harvested at 1, 4, and 7 days after the EdU pulse and the frequencies of EdU-positive FOB and MZB cells were measured with flow cytometry. One day after the pulse, there was ˜2-fold increase in the frequency of EdU-positive FOB cells in the spleens of Pacs1−/− mice compared to Pacs1+/+ mice, indicating a higher fraction of actively proliferating cells in the periphery. The frequency of EdU-positive FOB cells in Pacs1−/− spleens increased ˜3 to 4-fold by day 4 after the pulse, reflecting recruitment of immature B cells to the mature FOB population. EdU-labelled Pacs1−/− FOB cells decayed rapidly and were mostly lost by day 7. In contrast, there was ˜1.5 to 2-fold increase in the frequency of EdU-labeled Pacs1+/+ FOB cells on day 4 after the pulse that remained stable when analyzed on day 7. MZB cells were long lived with a slow turnover rate which was reflected in the low frequency of EdU+ cells in Pacs1+/+ and Pacs1−/− spleens at all time points. These data indicated that Pacs1−/− FOB cells had accelerated turnover rates and support observations herein of spontaneous B cell proliferation and apoptosis in the adoptive transfer assay.

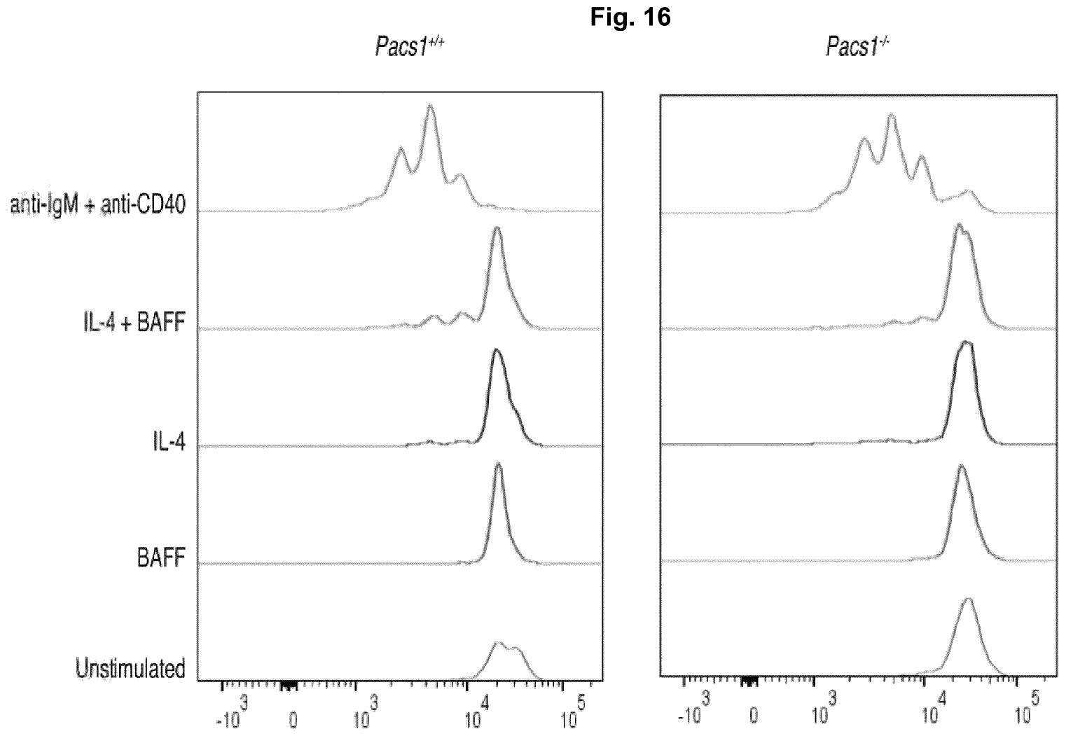

B cell populations are maintained in vivo by homeostatic cytokines like BAFF. To determine if spontaneous proliferation and increased turnover of Pacs1−/− B cells may be triggered by stimulation with these homeostatic cytokines, splenic B cells were harvested from Pacs1+/+ and Pacs1−/− mice and stimulated in vitro with BAFF and IL4, separately and together, for 72 hours (FIG. 16). Stimulation with anti-IgM and anti-CD40 was included as a positive control. While Pacs1^{+/+} and Pacs1^{-/-} B cells demonstrated normal proliferative responses to anti-IgM and anti-CD40, neither population showed significant proliferation after BAFF, IL-4, or combined treatment.

### Example 13. Pacs1 Deletion Suppressed Abnormal Lymphocyte Accumulation in Models of Lymphoproliferation

Defective lymphocyte cell-death pathways are crucial to the development of autoimmunity, lymphoproliferative diseases, and hematologic malignancies. The anti-apoptotic protein B cell lymphoma 2 (Bcl2) is frequently overexpressed in B cell malignancies and is a key contributor to tumorigenesis. Bcl2 overexpression blocks the mitochondrial apoptotic pathway both by inhibiting Bak and Bax oligomerization at the outer mitochondrial membrane by binding to IP3Rs to limit pro-apoptotic Ca2+ signals from the ER to the mitochondria. Based on the strong B cell depletion observed in Pacs1−/− mice, it was next investigated whether Pacs1 deletion might restore the ability of B cells to die in the context of forced Bcl2 expression.

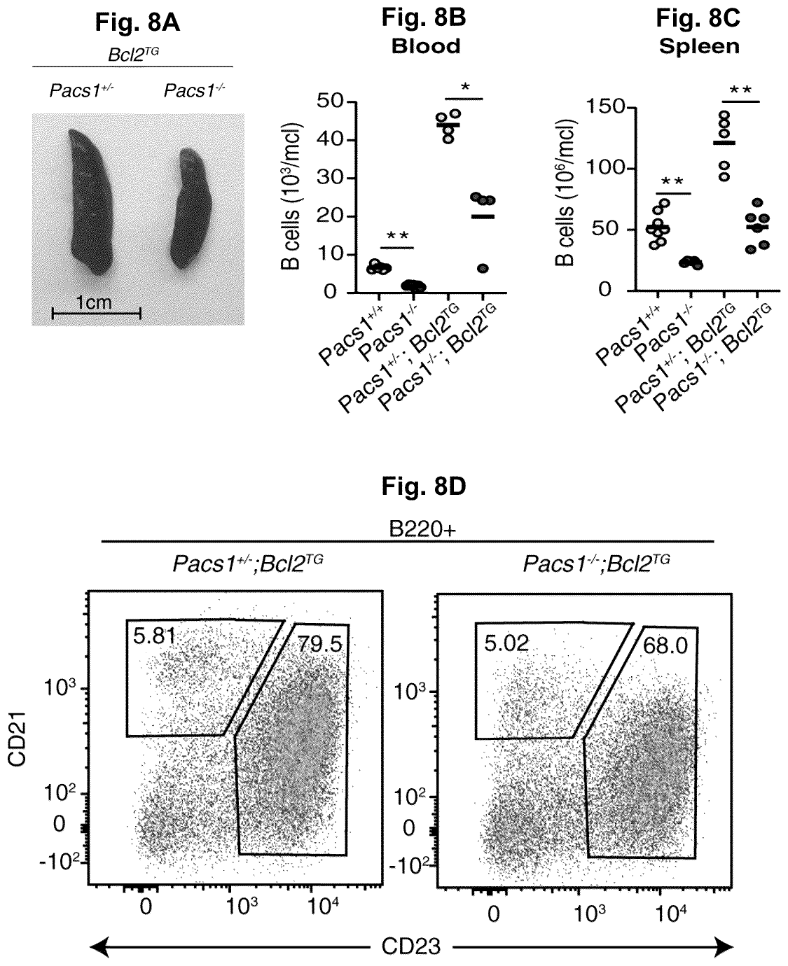

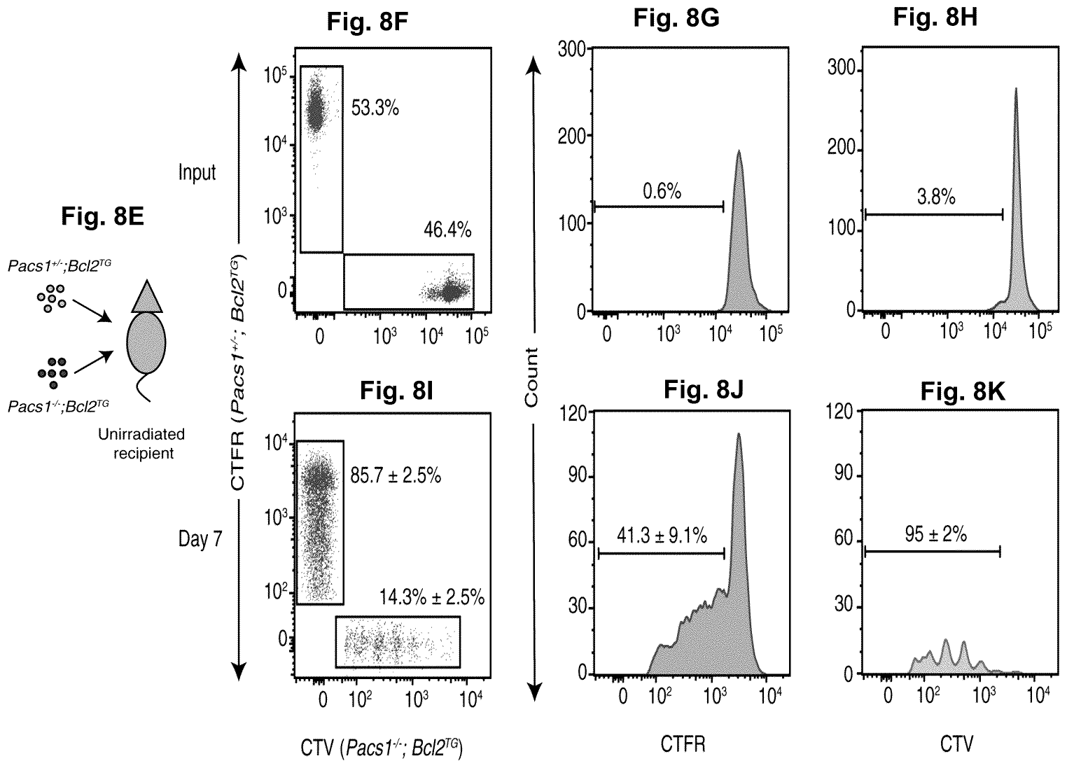

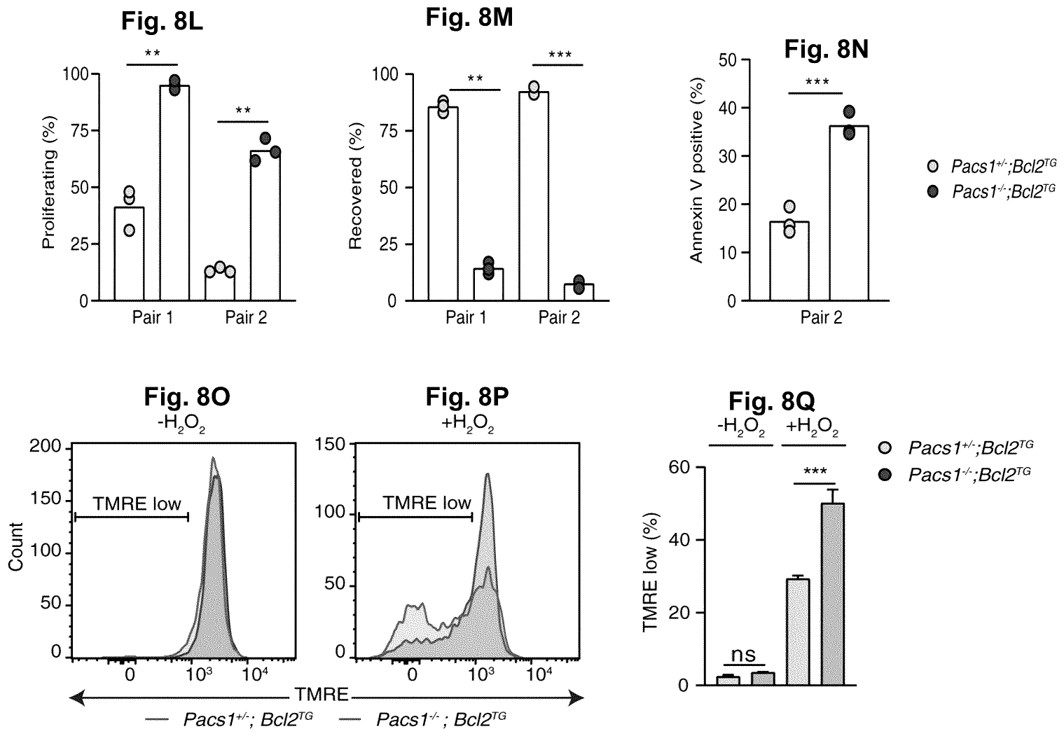

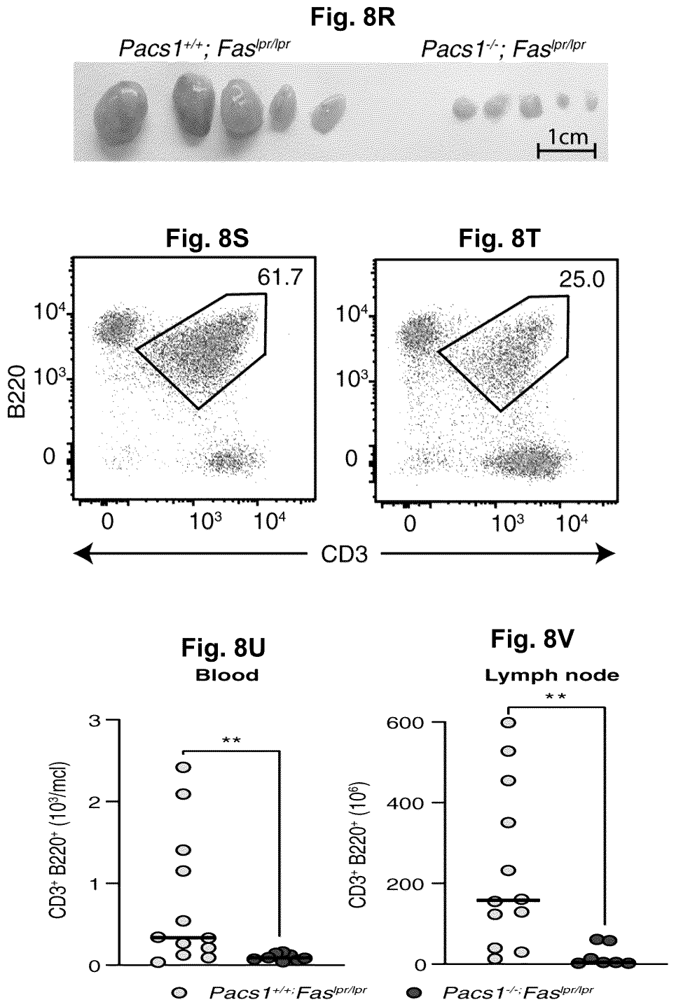

Mice overexpressing Bcl2 in the B cell lineage (Bcl2TG) developed abnormal expansion of FOB cells (FIG. 8A). Pacs1−/− mice were crossed to Bcl2TG mice and B cell counts were analyzed in the offspring at >20 weeks of age. Pacs1−/−; Bcl2TG mice showed reduced B cell counts in the peripheral blood and normalized splenic FOB cell counts compared to Pacs1+/−; Bcl2TG littermates (FIGS. 8B-8D), indicating that Pacs1 deletion could override the effects of Bcl2 in blocking B cell death.

To better understand how Pacs1 deletion could block expansion of Bcl2TG B cells, CTFR-labeled Pacs1+/−; Bcl2TG B cells and CTV-labeled Pacs1−/−; Bcl2TG B cells were transplanted into unirradiated CD45.1 recipients. Approximately 25-50% of Pacs1+/−; Bcl2TG cells showed spontaneous proliferation 7 days after transplant. In contrast, >95% of transferred Pacs1−/−; Bcl2-TG B cells underwent cell division (FIGS. 8E-8L). Additionally, Pacs1−/−; Bc12TG B cells were recovered at a much lower frequency compared to Pacs1+/−; Bcl2TG B cells from recipient spleens and showed higher rates of apoptosis (FIGS. 8M and 8N). Like Pacs1−/− cells, B cells isolated from the spleens of Pacs1−/−; Bcl2TG mice were more sensitive to oxidative stress after treatment with H₂O₂ (FIGS. 8O-8Q). These data indicated that Pacs1 deletion overrode the effect of forced Bcl2 expression by imparting increased sensitivity to cell death stimuli and diminishing lymphocyte quiescence.

It was observed that Pacs1 deletion caused both decreased T cell numbers and defective Ca2+ flux after TCR stimulation. Therefore, the effect of Pacs1 deletion on lymphoproliferative disease in the T cell lineage was assessed using the FasIpr model of lymphoproliferation. Loss-of-function Fas mutations in mice and humans leads to age-dependent expansion of an aberrant CD3+B220+ T cell population, which accumulate in large numbers in lymph nodes. Pacs1−/− mice to FasIpr/Ipr mice were crossed on the C57BL/6J background and peripheral blood and lymph node cell counts were monitored in aged mice (>24 weeks). A striking suppression of CD3+B220+ cells and bulky lymphadenopathy in Pacs1−/−; FasIpr/Ipr mice was observed compared with Pacs1+/+; FasIpr/Ipr mice (FIGS. 8R-8V). Together, these data indicated disruption of Pacs1-Wdr37 can potently suppress lymphoproliferative disease in B and T cell lineages arising from blocked cell-intrinsic and extrinsic apoptotic pathways.

Through forward genetic screening of randomly mutagenized mice, a known intracellular trafficking protein, phosphofurin acidic cluster sorting protein 1 (Pacs1), was found herein to be required for the development and survival of circulating lymphocytes due to its role in ER Ca²⁺ handling.

### Discussion of Examples 1-13

Through forward genetic screening and biochemical approaches it was found that Pacs1 and Wdr37 were required for normal lymphocyte homeostasis. The lymphocyte deficiency in Pacs1 and Wdr37 mice was linked to problems in ER Ca²⁺ handling. Pacs1 deletion resulted in decreased expression of IP3Rs and subsequently diminished Ca²⁺ emptying from the ER. Interestingly, Pacs1 deletion also resulted in low-level chronic ER Ca2+ leak. Pacs1−/− B cells showed elevated ER stress, oxidative metabolism, and ROS and were hypersensitive to oxidative stress in vitro. They also showed spontaneous loss of quiescence after adoptive transfer into lymphocyte replete recipients. Surprisingly, Pacs1−/− mice did not have major defects in immune competence. However, they were markedly resistant to lymphoproliferative diseases resulting from blocked cell-intrinsic or cell-extrinsic apoptotic pathways.

Reduced IP3R expression in Pacs1−/− cells. Decreased expression of all three IP3R isoforms was observed in Pacs1−/− B cells which blunted cytosolic Ca2+ flux after antigen receptor stimulation. IP3Rs were also downregulated when Pacs1 was deleted in 3T3 cells, suggesting a generally conserved mechanism. It was found that IP3R expression was reduced at the transcript level in both primary cells and 3T3 cells. Pacs1 deletion may modulate IPR3 gene expression by using downregulation of IPR3s as an adaptive response to chronic ER Ca2+ leak, increased ER stress, and ROS production that occurs after Pacs1 deletion to compensate ER Ca2+ depletion and disrupted proteostasis, a signal to the nucleus downregulates the ER Ca2+ flux machinery. This signal may be communicated by canonical ER stress or ROS signaling networks.

Pacs1-Wdr37 and ER Ca2+ leakage. In addition to causing the downregulation of IP3Rs, Pacs1 deletion also resulted in an increased rate of ER Ca2+ leakage. Without wishing to be bound by theory, a chronic ER Ca2+ leak in Pacs1−/− lymphocytes may have contributed to their elevated ER stress phenotype and their increased rates of cell death. The mechanism through which Pacs1-Wdr37 prevents ER Ca2+leakage may be that Pacs1-Wdr37 directly regulated the ER Ca2+ flux machinery. Indeed, it was found that SERCA2 was a candidate interacting protein in our Pacs1 interactome analysis. Additionally, IP3R receptors, among several classes of ion channels, contain Pacs protein binding motifs and are putative Pacs1 cargo molecules. Thus, Pacs1-Wdr37 could maintain ER Ca2+ content either by enhancing SERCA pump function or by blunting basal IP3R Ca2+ leak characteristics. Pacs1-Wdr37 disruption may increase ER stress more generally, for example, by disabling key steps in protein trafficking. Chronic ER stress can cause pro-apoptotic ER Ca2+ leak through increased in IP3R activity.

Loss of quiescence in Pacs1−/− B cells. Increased ER stress and ROS production in Pacs1−/− B cells likely contributed apoptosis at higher rates in vivo. Unexpectedly, Pacs1−/− B cells also spontaneously proliferated upon adoptive transfer into lymphocyte-replete recipients. Pacs1-B cells showed normal proliferative responses to antigen receptor signaling in vitro and did not spontaneously proliferate after stimulation with homeostatic cytokines. Chronic ER Ca2+ leak in Pacs1−/− cells may lead to a lower threshold for STIM-mediated SOCE and premature lymphocyte activation causing Pacs1−/− B cells to spontaneously proliferate. With elevated ER stress and ROS, and lack of second survival signals like CD40 or TLR stimulation, these cells would not be expected to survive long after activation. This model may explain another feature of Pacs1−/− lymphocytes. While decreased ER Ca2+ stores were measured after thapsigargin treatment, the magnitude of this difference was not as large as anticipated in cells with ongoing ER Ca2+ leakage. However, if lymphocytes with highly depleted ER stores are more prone to activation and cell death, it could be anticipated that a relatively small fraction remain in the total lymphocyte population at any one time. Rather, the population would be primarily composed of lymphocytes most adapted to retaining ER Ca2+, for example, by downregulating IP3Rs.

A novel mechanism for suppressing lymphoproliferative disease. Pacs1 deletion herein limited the expansion of lymphocytes in two clinically relevant models of lymphoproliferative disease affecting B cells (Bcl2 overexpression) and T cells (FasIpr). Exemplary methods herein indicated that Pacs1-Wdr37 maintained lymphocyte quiescence by supporting normal cellular Ca2+ homeostasis and reducing ER and oxidative stress. Overriding the quiescent state of diseased lymphocytes to force their elimination is a novel approach to the suppression of lymphoid diseases. Accordingly, Pacs1-Wdr37 is a viable therapeutic target for lymphoproliferative disease and possibly for lymphoid malignancies. Pharmacologic disruption of Pacs1-Wdr37 may synergize with existing therapies for hematologic malignancies that target lymphocyte survival factors such as Bcl2 (venetoclax), BTK (ibrutinib), and P13K (idelasib). Thus, Pacs1, Wdr37, and/or Pacs1-Wdr37 could limit lymphocyte expansion driven by other models of leukemogenesis such as c-Myc overexpression, p185 Bcr-Abl, or constitutive Notch activation.

Pacs1 and Wdr37 syndromes in humans. A spontaneous recurrent autosomal dominant mutation in the Pacs1 FBR (R203W) was identified as the causative genetic lesion in children with syndromic craniofacial abnormalities and intellectual disability. The disease-causing mechanism of Pacs1R203W is unclear and is currently thought to be a dominant negative or gain-of-function mutation. Similarly, subjects having variants of Wdr37 had symptoms associated with epilepsy, developmental delay, and cerebellar hypoplasia. Deficiency in the fly Wdr37 homolog had severe neurologic deficits that were not rescued by the human mutant variants. Neither Pacs1^{-/-} nor Wdr37^{-/-} mice had gross neurologic phenotypes. Testing effects of the mutant proteins on subcellular Ca²⁺ handling, ER and oxidative stress, and Pacs1, Wdr37, and Pacs1-Wdr37 complex formation can elucidate the pathophysiology of these human syndromes and define the role of Pacs1, Wdr37, and Pacs1-Wdr37 in neuronal function.

### Methods Used in Examples 1-13

Mouse strains. Mice were housed in specific pathogen-free conditions at the University of Texas Southwestern Medical Center and all experimental procedures were performed according to institutionally approved protocols. 8- to 10-week-old C57BLJ6J males were purchased from the Jackson Laboratory and mutagenized with ENU, similar to methods previously described (George et al., 2008). Strategic breeding of ENU-mutagenized generation 0 (G0) males, whole-exome sequencing, phenotypic screening, and automated mapping of G3 mice were performed similar to methods previously described (Wang et al., 2015). B6 CD45.1, Rag2−/−, FasIpr/Ipr, Ightm2Cgn (IgHB1-8i), and Tg(BCL2)22Wehi/J (Bcl2TG) mice were purchased from the Jackson Laboratory. Pacs1−/−; FasIpr/Ipr, Pacs1−/−; Bcl2TG, and Pacs1−/−; IgHB1-8/+ mice were generated by intercrossing mouse strains. Male and female mice aged 10-16 weeks were used for experiments. To elicit increased lymphocyte counts, mice on the FasIpr/Ipr and Bcl2TG backgrounds were aged longer (>20 weeks).

Generation of knockout mouse strains using the CRISPR/Cas9 system. To generate single knockout mouse strains, female C57BL/6J mice were super-ovulated by injection of 6.5 units (U) pregnant mare serum gonadotropin (PMSG; Millipore), followed by injection of 6.5 U human chorionic gonadotropin (hCG; Sigma-Aldrich) 48 hours later. The super-ovulated mice were subsequently mated overnight with C57BL/6J male mice. The following day, fertilized eggs were collected from the oviducts and in vitro-transcribed Cas9 mRNA (50 ng/mcl) and Pacs1, Pacs2, or Wdr37 small base-pairing guide RNA (50 ng/mcl; Pacs1: 5′-CATCTCGCTTAAGGAAATGA-3′ (SEQ ID NO: 1); Pacs2: 5′-ATGTGATCTCAAGACACGCT-3′ (SEQ ID NO: 2); Wdr37: 5′-GTGAAGGACAAGCGATCGAT-3′ (SEQ ID NO: 3)) were injected into the cytoplasm or pronucleus of the embryos. The injected embryos were cultured in M16 medium (Sigma-Aldrich) at 37° C. in 5% CO2. For the production of mutant mice, two-cell stage embryos were transferred into the ampulla of the oviduct (10-20 embryos per oviduct) of pseudo-pregnant Hsd:ICR (CD-1) female mice (Harlan Laboratories).

Plasmids. Mouse Pacs1 (amino acids 114-961), full-length mouse Wdr37, and full-length mouse SERCA2 were tagged with N-terminal FLAG or HA epitope in the pcDNA6 vector. Plasmids were sequenced to confirm the absence of undesirable mutations. Details of plasmids are available on request.

Immunizations and ELISA. For TD immunizations, mice were injected via the intraperitoneal route with 200 mcg ovalbumin or 100 mcg NP-KLH (BioSearch) adsorbed on aluminum hydroxide hydrogel (InvivoGen). For TI immunizations, mice were given intraperitoneal injections of 50 mcg TNP-Ficoll (BioSearch). At the indicated time points, peripheral blood was harvested in MiniCollect tubes (Mercedes Medical) and centrifuged at 10,000 rpm to separate the serum for ELISA analysis. For high and low affinity antibody detection, Nunc MaxiSorp flat-bottom 96-well microplates (Thermo Fisher Scientific) were coated with 5 mcg/ml NP2-BSA or NP30-BSA (BioSearch). Plates were washed four times using a BioTek microplate washer and then blocked with 1% (v/v) bovine serum albumin (BSA) in PBS for 1 hour at room temperature (25° C.±3° C.). Serum from immunized mice was serially diluted in the prepared ELISA plates. After 2 hours of incubation, plates were washed eight times with washing buffer and then incubated with horseradish peroxidase (HRP)-conjugated goat anti-mouse IgG (Thermo) for 1 hour at room temperature. Plates were washed eight times with washing buffer and then developed with SureBlue TMB Microwell Peroxidase Substrate and TMB Stop Solution (KPL). Absorbance was measured at 450 nm on a Synergy Neo2 plate reader (BioTek).

Bone marrow chimera and adoptive transfer experiments. At 24 hours prior to transplant, Rag2−/− recipient mice were lethally irradiated with 11 Gy given in split doses (X-RAD 320, Precision X-ray). Bone marrow was flushed from the tibias and fibulas from the indicated donor strains. Red blood cells were lysed in RBC lysis buffer (BD Biosciences) and bone marrow cells were counted and combined at a 1:1 ratio. Approximately 5-6 million cells were injected intravenously via the retro-orbital route into Rag2−/− recipients. Recipient mice were maintained on antibiotic water for 8 weeks post-transplant. At 16 weeks after transplant, primary and secondary lymphoid tissues were harvested to assess donor chimerism based on lineage, CD45.1, and CD45.2 staining. For B cell adoptive transfer, B cells were purified to >90% purity from the spleen of indicated donor strains (pan-B isolation kit; StemCell Technologies). Cells were stained with CTFR or CTV proliferation dyes (Molecular Probes) according to the manufacturer's instructions. Differentially labeled cells were combined a 1:1 ratio and 3-4 million cells were injected intravenously into unirradiated CD45.1 recipients. At 7-8 days after transplant, spleens from the recipient mice were harvested. The frequency and proliferation status of donor cells was assessed based on positive staining for CD45.2 and the fluorescence of the proliferation dyes.

Transfection, co-immunoprecipitation, and western blotting. HEK293T cells were maintained in DMEM containing 10% FBS. Cells were transfected in 6-well plates with 2 mcg of the indicated constructs in the presence of Lipofectamine 2000 according to the manufacturer's instructions. At 36-48 h post-transfection, cells were rinsed in cold PBS and lysed in buffer containing 1% NP-40 and HALT protease inhibitor (Thermo). Immunoprecipitation of FLAG-tagged proteins was performed by incubating M2 anti-FLAG resin (Sigma) with cell lysates for 2 h at 4° C. with end-over-end rotation. Beads were washed four times in cold lysis buffer and protein complexes were eluted with 150 mg/ml of 3×FLAG peptide (Sigma). Samples were diluted in 2×SDS sample buffer, run on SDS-PAGE, and transferred to nitrocellulose membranes according to standard procedures. For western blotting on primary cells, cell pellets were lysed in buffer containing 1% SDS and HALT protease inhibitor. Protein levels were normalized using the bicinchoninic acid (BCA) assay (Pierce) and 10-15 mcg of protein was diluted in 2×SDS sample buffer and run on SDS-PAGE.

Generation of Pacs1 knockout NIH-3T3 cell lines. NIH-3T3 cells (ATCC) were transfected with pSpCas9(BB)-2A-GFP (PX458) encoding a small base-pairing guide RNA targeting the genomic locus of mouse Pacs1 (5′-CATCTCGCTTAAGGAAATGA-3′ (SEQ ID NO: 1)). Forty-eight hours after transfection, GFP+cells were sorted by flow cytometry and single colonies were selected by limiting dilution. Clonal cell lines were screened for Pacs1 deletion by immunoblotting.

Lymphocyte Ca2+ flux measurements. Splenocytes were harvested from the indicated strains and RBCs were lysed. Cells were loaded for 30 minutes at 37° C. with Indo-1, AM (Molecular Probes) according to manufacturer's instructions in RPMI containing 2% FBS (R2). After dye loading, cell surface staining with fluorescence conjugated antibodies to resolve T and B cell subsets was performed on ice for 20 minutes. Cells were washed once in cold PBS and resuspended at 10 million cells/ml in cold R2. To measure Ca2+flux, 100 mcl of cells were diluted into 900 mcl of warm R2 and incubated at 37° C. for 2 minutes. Indo-1 fluorescence was then measured with flow cytometry in response to treatment with anti-IgM (Invitrogen). For Ca2+store mobilization, 100 mcl of labeled splenocytes was diluted in 900 mcl of warm HBSS containing 1 mM EGTA and 10 mM HEPES and incubated at 37° C. for 2 minutes.

Sub-cellular Ca2+ measurements. For cytosolic AEQ (cytAEQ), the coverslip containing transfected cells was incubated with 5 mcM coelenterazine for 1-2 h in KRB (Krebs-Ringer modified buffer: 125 mM NaCl, 5 mM KCI, 1 mM Na3PO4, 1 mM MgSO4, 5.5 mM glucose, and 20 mM Hepes, pH 7.4, at 37° C.) supplemented with 1% FCS, and then transferred to the perfusion chamber. For reconstituting the AEQ chimeras targeted to the ER (erAEQ) with high efficiency, luminal Ca2+must first be reduced. This was achieved by incubating cells for 1 h at 4° C. in KRB supplemented with coelenterazine 5 mcM, ionomycin, and 600 mcM EGTA. After this incubation, the cells were extensively washed with KRB supplemented with 2% BSA and 1 mM EGTA. All AEQ measurements were carried out in KRB and all agonists and other drugs were also dissolved in KRB. The experiments were terminated by lysing cells with 100 mcM digitonin in a hypotonic solution containing 10 mM CaCl2, thus discharging the remaining AEQ pool. The light signal was collected and calibrated into [Ca2+] values similar to methods previously described (Bonora et al., 2013).

Ca2+ imaging experiments were performed similar to methods previously described (Filippin et al., 2003; Patron et al., 2014). Briefly, cells were transfected with 2mtGCaMP6m or ER-GCaMP6-210 encoding plasmids and transferred to glass coverslips 24h post-transfection. Where indicated, cells were infected with an ecotropic retrovirus encoding Mito-Pericam (pMSCVpuro-Mito-Pericam). Imaging was performed in HBSS supplemented with 1 mM CaCl2, 1% FCS, and 20 mM HEPES, pH 7.4 at 37° C. Images were obtained on a wide-field fluorescence microscope with a high magnification oil immersion lens (40× or 60×, n.a. 1.4). Cells were alternatively illuminated at 474 nm and 410 nm and fluorescence collected through a 515/30-nm band-pass filter. Analysis was performed with the Fiji open source software. Both images were background corrected frame by frame by a rolling ball algorithm, then manually thresholded to select for positive pixels. Data are presented as the mean of the averaged ratio of all time points.

Proteomics. FLAG-Pacs1 was transfected into HEK 293T cells. At 48 hours after transfection, cells were lysed in buffer containing 1% NP-40, and FLAG-Pacs1 was purified with M2 anti-FLAG resin. Bead-bound FLAG-Pacs1 was washed four times in lysis buffer and incubated with primary B cell extract in 1% NP-40 lysis buffer overnight at 4° C. As a negative control, FLAG beads were incubated with B cell extract in 1% NP-40 lysis buffer overnight at 4° C. Co-immunoprecipitates were washed four times in lysis buffer, eluted with 150 mg/ml 3×FLAG peptide, and diluted in 6×SDS sample buffer. Samples were run on SDS-PAGE until they had entered ˜0.5 cm into the resolving gel. Protein was visualized with Gel-Code Blue (Thermo), cut from the gel, and submitted to the UT Southwestern Proteomics Core for LC-MS/MS analysis similar to methods previously described (Zhang et al., 2016). Data was semi-quantified based on peptide spectrum matches (PSM) and candidate binding proteins were ranked based on the PSM ratio of FLAG-Pacs1/beads.

In vitro lymphocyte studies. For proliferation assays, B cells were purified from the spleens of the indicated strains (pan-B isolation kit; StemCell Technologies) and labeled with CTV. Labelled cells were incubated at a concentration of 1 million cells/ml in 24-well plates in X-VIVO 15 (Lonza) supplemented with 2-mercaptoethanol, glutamine, and antibiotics. Cells were treated with indicated amounts of anti-IgM (Invitrogen), anti-CD40 (Mitenyi), LPS (Enzo), murine IL4 (Biolegend), or murine BAFF (Peprotech). Proliferation was measured 72 hours post-stimulation with FACS analysis based on CTV dilution. For oxidative cell death studies, splenocytes from Pacs1+/+ and Pacs1−/− mice were stained on ice to identify FOB cells then washed in PBS and re-suspended in culture media. Approximately 1 million cells were then treated with 100 mcM H₂O₂ (Sigma) at 37° C. for 35 minutes followed staining with 30 nM TM RE for an additional 15 minutes. TM RE fluorescence was measured using FACS analysis. For ROS analysis, approximately 1 million splenocytes were stained on ice to identify FOB cells, washed with PBS, then incubated with CelIRox Green (Molecular Probes) according to the manufacturer's protocol. For oxygen consumption studies, purified splenic B cells were left alone or stimulated overnight with anti-IgM followed by metabolic flux analysis using either an XFe96 or XFe24 machine according to published protocols. Oxygen consumption rates were normalized to total cells plated.

Statistical analysis. Normal distribution of data was determined by the Shapiro-Wilk normality test. For normally distributed data, the statistical significance of differences between experimental groups was determined by Student's unpaired t test. Paired t tests were performed to compare Ca2+ flux and ROS data and are indicated by lines connecting paired data points. For non-normally distributed data a non-parametric test was used as indicated. Statistical analysis was performed using GraphPad Prism software. Differences with P values <0.05 were considered significant. P values are denoted by *P <0.05, **P<0.01, and ***P<0.001. Differences with P values 0.05 were considered not significant (ns).

### REFERENCES CITED IN EXAMPLES 1-13

Bohler et al., (2018) Cell Death Dis 9: 286;
Bonora et al., (2013) Nat Protoc 8: 2105-2118;
Filippin et al., (2003) J Biol Chem 278: 39224-39234;
Georgel et al., (2008) Methods Mol Biol 415: 1-16;
Patron et al., (2014) Mol Cell 53: 726-737;
Wang et al., (2015) Proc Natl Acad Sci USA 112: E440-E449; and
Zhang et al., (2016) Proc Natl Acad Sci USA 113: E6418-E6426.

1. A method for attenuating or preventing lymphoproliferation in a subject in need thereof, the method comprising administering to the subject a composition effective for modulating WD repeat domain 37 (Wdr37),
wherein modulating Wdr37 comprises decreasing Wdr37 gene expression, decreasing Wdr37 protein expression, decreasing Wdr37 activity, or any combination thereof.

2. The method of claim 1, wherein the composition effective for modulating Wdr37 comprises at least one of a peptide, an antibody, a chemical, a compound, an oligo, a nucleic acid molecule, or a combination thereof.

3. The method of claim 2, wherein the nucleic acid molecule comprises a double-stranded RNA effective for inhibiting or decreasing the expression of Wdr37.

4. The method of claim 3, wherein the double-stranded RNA is selected from the group consisting of small temporal RNA, small nuclear RNA, small nucleolar RNA, short hairpin RNA and microRNA.

5. The method of claim 4, wherein the double-stranded RNA is a small interfering RNA.

6. The method of any ono of claims 1 5 claim 1, wherein the composition effective for modulating Wdr37 further comprises at least one pharmaceutically acceptable excipient.

7. The method of claim 1, wherein the subject administered the composition effective for modulating Wdr37 is a subject having, suspected of having, or at risk of having at least one lymphoproliferative disease, at least one lymphoid malignancy, or a combination thereof.

8. The method of claim 7, wherein the subject having, suspected of having, or at risk of having at least one lymphoproliferative disease, is a human subject having one or more genetic markers for a lymphoproliferative disorder.

9. The method of claim 8, wherein the human subject having one or more genetic markers for a lymphoproliferative disorder comprises a human subject that has been diagnosed as having or is suspected of having autoimmune lymphoproliferative syndrome (ALPS), Castleman disease (CD), Rosai-Dorfman disease (RDD), EBV-associated lymphoproliferative disorder (ELD), X-linked lymphoproliferative syndrome (XLP), angioimmunoblastic lymphadenopathy, caspase-8 deficiency syndrome (CEDS), Dianzani autoimmune lymphoproliferative disease, Kikuchi-Fujimoto syndrome, Llymphomatoid granulomatosis, lymphomatoid papulosis, ocular adnexal lymphoid proliferation, RAS-associated leukoproliferative disorder (RALD), p110δ activating mutation causing senescent T cells lymphadenopathy and immunodeficiency (PASLI), CTLA-4 haploinsufficiency with autoimmune infiltration (CHAT), LRBA deficiency with autoantibodies, regulatory T-cell defects, autoimmune infiltration and enteropathy (LATAIE), X-linked immunodeficiency with magnesium defect, EBV infection, and neoplasia (X-M EN), interleukin-2-inducible T-cell kinase (ITK) deficiency, or any combination thereof.

10. The method of claim 1, wherein the subject administered the composition effective for modulating Wdr37 is an immunocompromised subject.

11. The method of claim 10, wherein the immunocompromised subject comprises a human immunocompromised subject that has been diagnosed as having or is suspected of having common variable immunodeficiency (CVID), severe combined immunodeficiency (SCID), Wiskott-Aldrich syndrome, ataxia-telangiectasia, Chediak-Higashi syndrome, one or more viral infections, one or more fungal infections, or any combination thereof.

12. The method of claim 11, wherein the human immunocompromised subject is diagnosed as having or is suspected of having human immunodeficiency virus (HIV), severe acute respiratory syndrome coronavirus 1 (SARS-CoV-1), severe acute respiratory syndrome coronavirus 2 (SARS-CoV-2), Middle East Respiratory Syndrome (MERS), human coronavirus OC43 (HCoV-0043), human coronavirus HKU1 (HCoV-HKU1), human coronavirus 229E (HCoV-229E), human coronavirus NL63 (HCoV-NL63), or any combination thereof.

13. The method of claim 7, wherein the subject having, suspected of having, or at risk of having at least at least one lymphoid malignancy comprises a human subject having at least one lymphoid malignancy selected from the group comprising Hodgkin lymphomas, non-Hodgkin lymphomas, mature B cell neoplasms, mature T cell and natural killer (NK) cell neoplasms, and precursor lymphoid neoplasms.

14. The method of claim 1, wherein the composition effective for modulating Wdr37 is administered to the subject topically, systemically, subcutaneously, intravenously, or intranasally.

15. The method of claim 1, wherein the subject has undergone or is undergoing at least one other therapy for lymphoproliferation.

16. The method of claim 15, wherein the at least one other therapy for lymphoproliferation comprises administration of chemotherapy, rituximab, obinutuzumab, bortezomib, carfilzomib, azacitidine, decitabine, venetoclax, ibrutinib, idelalisib, sunitinib, dinaciclib, cobimetinib, idasanutlin, oblimersen sodium, sodium butyrate, depsipeptide, fenretinide, flavopiridol, gossypol, ABT-737, ABT-263, GX15-070, HA14-1, Antimycin A, acalabrutinib, zanubrutinib, tirabrutinib, bortezomib, lenalidomide, temsirolimus, or any combination thereof.

17. A composition comprising at least one inhibitor of WD repeat domain protein 37 (Wdr37), and a pharmaceutically acceptable carrier.

18. The composition of claim 17 further comprising at least one pharmaceutically acceptable excipient.

19. The composition of claim 17, wherein the at least one inhibitor of Wdr37 comprises at least one of a peptide, an antibody, a chemical, a compound, an oligo, a nucleic acid molecule, or a combination thereof, and
wherein the at least one inhibitor of Wdr37 inhibits Wdr37 direct activity, inhibits Wdr37 indirect activity, inhibits formation of a complex between Wdr37 and phosphofurin acidic cluster sorting protein 1 (Pacs1), decreases expression of the Wdr37 gene, decreases expression of the Wdr37 protein, or any combination thereof.

20. The composition of claim 19, wherein the at least one inhibitor of Wdr37 comprises a nucleic acid molecule comprising a double-stranded RNA effective for inhibiting or decreasing the expression of Wdr37.

21. The composition of claim 20, wherein the double-stranded RNA is selected from the group consisting of small temporal RNA, small nuclear RNA, small nucleolar RNA, short hairpin RNA and microRNA.

22. The composition of claim 21, wherein the double-stranded RNA is a small interfering RNA.

23. A method for treating at least one lymphoproliferative disease, at least one lymphoid malignancy, or a combination thereof in a subject, the method comprising administering to a subject in need thereof an effective amount of the composition of claim 17.

24. The method of claim 23, wherein the subject is a human subject having, suspected of having, or at risk for at least one lymphoproliferative disease, at least one lymphoid malignancy, or any combination thereof.

25. The method of claim 23 further comprising administering to the subject an effective amount of at least one therapy for lymphoproliferation.

26. The method of claim 25, wherein the at least one therapy for lymphoproliferation comprises chemotherapy, rituximab, obinutuzumab, bortezomib, carfilzomib, azacitidine, decitabine, venetoclax, ibrutinib, idelalisib, sunitinib, dinaciclib, cobimetinib, idasanutlin, oblimersen sodium, sodium butyrate, depsipeptide, fenretinide, flavopiridol, gossypol, ABT-737, ABT-263, GX15-070, HA14-1, Antimycin A, acalabrutinib, zanubrutinib, tirabrutinib, bortezomib, lenalidomide, temsirolimus, or any combination thereof.

27. A kit comprising a composition effective for modulating WD repeat domain protein 37 (Wdr37), and at least one container,
wherein modulating Wdr37 comprises decreasing Wdr37 gene expression, decreasing Wdr37 protein expression, decreasing Wdr37 activity, or any combination thereof.
